SEQUENCE LISTING (14 sequences)

SEQ ID NO: 1 - RNA, 21 nt, Artificial Sequence
  source: /note=“Description of Artificial Sequence Synthetic oligonucleotide”
acaccaaucuccucaacgauu

SEQ ID NO: 2 - RNA, 21 nt, Artificial Sequence
  source: /note=“Description of Artificial Sequence Synthetic oligonucleotide”
ucguugaggagauugguguuu

SEQ ID NO: 3 - RNA, 21 nt, Artificial Sequence
  source: /note=“Description of Artificial Sequence Synthetic oligonucleotide”
gcauguucguggccucuaauu

SEQ ID NO: 4 - RNA, 21 nt, Artificial Sequence
  source: /note=“Description of Artificial Sequence Synthetic oligonucleotide”
uuagaggccacgaacaugcuu

SEQ ID NO: 5 - RNA, 21 nt, Artificial Sequence
  source: /note=“Description of Artificial Sequence Synthetic oligonucleotide”
gccgagaaguugugcacuuuu

SEQ ID NO: 6 - RNA, 21 nt, Artificial Sequence
  source: /note=“Description of Artificial Sequence Synthetic oligonucleotide”
agaugcacaacuucucggcuu

SEQ ID NO: 7 - RNA, 21 nt, Artificial Sequence
  source: /note=“Description of Artificial Sequence Synthetic oligonucleotide”
gcacuuucuuuccagagucuu

SEQ ID NO: 8 - RNA, 21 nt, Artificial Sequence
  source: /note=“Description of Artificial Sequence Synthetic oligonucleotide”
gacucuggaaagaaagugcuu

SEQ ID NO: 9 - DNA, 22 bp, Artificial Sequence
  source: /note=“Description of Artificial Sequence Synthetic primer”
ctcacatctgctgctccacaag

SEQ ID NO: 10 - DNA, 23 bp, Artificial Sequence
  source: /note=“Description of Artificial Sequence Synthetic primer”
aatttggtgcttcacacttcagg

SEQ ID NO: 11 - DNA, 20 bp, Artificial Sequence
  source: /note=“Description of Artificial Sequence Synthetic primer”
cctgtagcccacgtcgtagc

SEQ ID NO: 12 - DNA, 20 bp, Artificial Sequence
  source: /note=“Description of Artificial Sequence Synthetic primer”
ttgacctcagcgctgagttg

SEQ ID NO: 13 - DNA, 20 bp, Artificial Sequence
  source: /note=“Description of Artificial Sequence Synthetic primer”
cttcctctccaaaatgccag

SEQ ID NO: 14 - DNA, 20 bp, Artificial Sequence
  source: /note=“Description of Artificial Sequence Synthetic primer”
agagatggaagggggaaaga

(19) United States
(12) Patent Application Publication
(10) Pub. No.: US 2011/0177155 A1
(43) Pub. Date: Jul. 21, 2011

(54) METHODS OF DELIVERY OF AGENTS TO LEUKOCYTES AND ENDOTHELIAL CELLS

(75) Inventors: Dan Peer, Kiryat Ono (IL); Motomu Shimaoka, Brookline, MA (US)

(73) Assignee: IMMUNE DISEASE INSTITUTE, INC., Boston, MA (US)

(21) Appl. No.: 12/674,504

(22) PCT Filed: Aug. 20, 2008
(86) PCT No.: PCT/US2008/073658

§ 371 Date: Feb. 22, 2010

Related U.S. Application Data
(60) Provisional application No. 60/957,023, filed on Aug. 21, 2007.

(51) Int. Cl. A61K 9/127; C12N 5/00; C12N 5/071

(52) U.S. Cl. 424/450; 435/325; 435/366

(57) ABSTRACT

The present invention generally relates to methods and compositions for the simultaneous delivery of at least one insoluble agent and at least one soluble agent to a cell. In particular the present invention relates to methods and compositions for the dual delivery of an insoluble agent and a soluble agent to a particular target cell, for example, a leukocyte or endothelial cell. In particular, methods and compositions for simultaneous delivery of a hydrophilic (i.e. soluble) agent and/or a hydrophobic (i.e. insoluble) agent to a leukocyte cell or endothelia cell are disclosed.

### CROSS REFERENCED APPLICATIONS

This application claims benefit under 35 U.S.C. 119(e) of U.S. Provisional Patent Application Ser. No. 60/957,023 filed on Aug. 21, 2007, the contents of which are incorporated herein in its entity by reference.

### GOVERNMENT SUPPORT

The present application was made with Government support under Grant No: RO1 AI063421 awarded by the National Institutes for Health (NIH). The Government of the United States has certain rights thereto.

### FIELD OF THE INVENTION

The present invention relates generally to the field of drug delivery, and more particularly to the simultaneous delivery of soluble and insoluble agents to target cells, such as leukocytes and endothelial cells.

### BACKGROUND OF THE INVENTION

### Leukocytes and Endothelial Cells are Target for a Wide Range of Important Pathologies Such as Inflammation and Cancers.

The vascular endothelial cells form a continuous, single monolayer that lines the vascular system, within which leukocytes travel. Interactions of leukocytes and endothelial cells are tightly regulated so that leukocytes adhere to and transmigrate through endothelial cells only upon activation of appropriate signaling cascade(s). However, aberrant regulation of leukocytes and endothelial cells results in accumulation of leukocytes and platelets, extravasation of leukocytes, and increased vascular permeability. Dysregulation of leukocytes and endothelial cells lead to sustained inflammation and tissue damages as seen in autoimmune diseases, ischemia-reperfusion injury, atherosclerosis, and pathological angiogenesis.

Leukocyte and endothelial molecules involved in the pathogenesis of inflammatory tissue damages have been extensively studied, including cell adhesion molecules, cell surface receptors, and secreted cytokines. Inhibition of cell adhesion molecules and cytokines and their receptors has been shown to suppress animal disease models. However, in clinical trials, many blocking antibodies failed to show clinical efficacies, at least partly because the pathogenesis of human inflammatory and autoimmune diseases involves multiple inflammatory pathways including not only cell adhesion molecules and cytokines but also intracellular signaling molecules that orchestrate and amplify inflammatory cascades.

Small-molecule drugs including small-molecule chemicals and nucleic acids (e.g. siRNAs) are promising candidates to manipulate each inflammatory cascade individually as well as multiple cascades simultaneously. In contrast to antibodies that usually block cell surface receptors and secreted molecules, an appropriate choice of small-molecule drugs can allow for blocking extracellular and/or intracellular targets. However, small-molecule drugs do not usually have good pharmacokinetic profiles as antibodies. In addition, cellular uptake of small-molecule drugs is usually not selective and controllable, and, in the case of siRNA, not effective.

Therefore, in order to develop small-molecule drugs into effective therapeutics, a novel platform technology is required to efficiently and selectively deliver small-molecule drugs to targets (i.e. leukocytes and endothelial cells).

Integrins and their Ligands are Important for Leukocyte-Endothelial Cell Interactions at the Site of Inflammation and for Leukocyte Extravasation.

By binding to their Immunoglobulin super family (IgSF) ligands on endothelial cells, integrins play an important role in mediating leukocyte-endothelial cell interactions at sites of inflammation. Blocking antibodies to integrins on leukocytes and integrin IgSF ligands on endothelial cells have been shown to inhibit infiltration of leukocytes at inflamed tissues and reduce tissue damages in a wide range of inflammatory disease models.

At least 18 α-subunits and 8 β-subunits of integrins have been identified thus far, forming at least 24 different integrin heteromiders that are expressed in various types of cells. However, β2 integrins (α_{L}β₂, α_{M}β₂, α_{X}β₂, α_{D}β₂) and β₇ integrins (α₄β₇, and α_{E}β₇) are exclusively expressed on leukocytes. In adults, α₄β₁ is also exclusively expressed on leukocytes, as is integrin LFA-1 (α_{L}β₂). Integrin ligands are expressed on endothelial cells, and the interaction of the integrin on the leyokcyte and integrin ligand or cellular adhesion molecules (CAM) on the endothelial cell enable leukocyte diapedesis and transendothelial migration of the leukocyte and leukocyte infiltration into an area of tissue injury or inflammation. Although healthy endothelial cells express basal levels of ICAM-1 (intracellular adhesion molecule 1), the expression of ICAM-1 is upregulated by inflammation. Whereas little VCAM-1 (vascular cell adhesion molecule 1) is expressed in healthy endothelial cells, the expression of VCAM-1 is upregulated by inflammation and vascular injury such as atherosclerosis. The expression of MAdCAM-1 is limited to the gut, and upregulated in the gut by colitis.

A critical obstacle and challenge for any therapeutic treatment is the limited availability of effective biocompatible delivery systems. Since many therapeutic agents work in conjunction, either synergistically or additive with other agents, effective delivery systems for the simultaneous delivery of more than one agent to a target cell is advantageous for improved therapeutic efficacy. However, many of agents which function synergistically with another agent cannot be delivered simultaneously due to differences in solubility. Often for insoluble agents to be used on humans, such as CPT or Taxol, which are poorly water soluble cancer therapies, the agents are often mixed with organic solvents in order to be delivered into the body, which can cause toxic side effects and the potential to decrease the potency of the cancer therapy. Accordingly, there is a need for effective delivery systems for simultaneous delivery of agents which have different solubility to target cells.

### SUMMARY OF THE INVENTION

The present invention relates to methods and compositions for the simultaneous delivery of at least one insoluble agent and at least one soluble agent to a cell. In some embodiments, the inventors have discovered methods and compositions for the delivery of an insoluble agent and a soluble agent to a target cell. In some embodiments, the target cell is a leukocyte. Accordingly, the inventors have discovered a method to deliver at least one hydrophilic agent and at least one hydrophobic agent to a leukocyte, by contacting the leukocyte with a carrier particle comprising an insoluble agent (or hydrophobic agent) and a soluble agent (hydrophilic agent), wherein the carrier particle is coated with a targeting moiety specific to a targeting the carrier particle to the leukocyte.

In some embodiments, the targeting moiety is any entity, such as proteins, antibodies, antibody fragments and the like which bind or have affinity for integrins expressed on the cell surface of the target cell. In embodiments where the target cell is a leukocyte, a targeting moiety can be an antibody or fragment thereof that binds to an integrin present on the cell surface of the leukocyte. Alternatively, where the target cell is a leukocyte, a targeting moiety can be an integrin ligand or fragment thereof that binds to an integrin present on the cell surface of the leukocyte. Accordingly, aspects of the present invention relate to the inventor's discovery of a method to simultaneously deliver a hydrophilic (i.e. soluble) agent and/or a hydrophobic (i.e. insoluble) agent to a leukocyte cell, by entrapping the soluble and insoluble agents in a carrier particle and coating the carrier particles with targeting moieties which direct delivery of the insoluble and soluble agents to the leukocyte target cell. While one can deliver at least one insoluble agent or at least one soluble agent to the target cell, such as a leukocyte, a preferred embodiment is the simultaneous delivery of both an insoluble agent and a soluble agent to the target cell, such as leukocyte.

Accordingly, one aspect of the present invention relates to a composition for the simultaneous delivery of an insoluble agent and a soluble agent to a target cell, wherein the composition comprises a carrier particle comprising an insoluble agent and/or a soluble agent, wherein the carrier particle is attached to a targeting moiety, where the targeting moiety binds to and has specific affinity to a cell surface marker on the target cell.

In some embodiments, using the compositions and methods as disclosed herein one can deliver an insoluble agent and/or a soluble agent to any target cell. The target cell can be any cell from any species, for example mammalian species, and in some embodiments humans. One can target any cell or target any cell type in which is desired to have the simultaneous delivery of an insoluble agent and a soluble agent. As exemplary demonstration of delivery to target cells, but by no means limitation, the inventors have demonstrated delivery to leukocytes and endothelial cells. As disclosed herein and in Example 7, the inventors demonstrate targeted delivery of an insoluble agent (TAXOL®) and a soluble agent (RNAi) to leukocytes using either anti-integrin antibody-coated carrier particles or integrin ligand-coated carrier particles. The inventors have also demonstrated targeted delivery of an insoluble agent and/or a soluble agent to endothelial cells using anti-integrin ligand antibody-coated carrier particles or integrin-coated carrier particles.

Accordingly, one aspect of the present invention relates to, in part, a method to deliver an agent to leukocytes. In particular, the inventors have discovered a method to deliver at least one agent, for example a hydrophilic agent and/or a hydrophobic agent to leukocytes. The inventors have discovered a method to deliver agents to leukocytes by associating a targeting moiety to a carrier particle, where the targeting moiety has affinity for, or binds to integrins present on the surface of leukocytes, and where an agent is associated with the carrier particle. Accordingly, the present invention relates to a leukocyte delivery agent, comprises a targeting moiety that has affinity for integrins on leukocytes, where the targeting moiety is associated with a carrier particle.

One aspect of the invention relates to a leukocyte delivery agent, comprises a targeting that has affinity for, or binds to integrin expressed in leukocytes, for example but not limited to of LFA-1 (αLβ2), Mac-1 (αMβ2), p150.95 (αXβ2), αDβ2, VLA-4 (α4β1), and β₇ (α4β7 and αEβ7). In some embodiments, such a targeting moiety is an antibody or fragment thereof with affinity for, or binds LFA-1 (αLβ2), Mac-1 (αMβ2), p150.95 (αXβ2), αDβ2, VLA-4 (α4β1), and β₇ (α4β7 and αEβ7). In alternative embodiments, a targeting moiety can be an integrin ligand or fragment or variant or homologue thereof that binds to integrins; LFA-1 (αLβ2), Mac-1 (αMβ2), p150.95 (αXβ2), VLA-4 (α4β1), and β₇ (α4β7 and αEβ7). Examples of such integrin ligands useful as targeting moieties of the present invention include, for example but are not limited to members of the IgSF (Ig Superfamily) of cell Adhesion molecules (CAM) expressed on endothelial cells, for example, ICAM-1, ICAM-2, ICAM-3, VCAM-1, MAdCAM-1, E-cadherin, JAM-1, JAM-2, JAM-3 or fragments, homologues or variants thereof.

Another aspect of the present invention relates to a method to deliver agents to endothelial cells, and an endothelial delivery agent to deliver agents to endothelial cells, the method and agent comprising a targeting moiety that has affinity for integrin ligands on endothelial cells, where the targeting moiety is associated with a carrier particle. In such embodiments, such a endothelial targeting moiety that has affinity for, or binds to integrin ligands expressed on endothelial cells, for example but not limited to members of the IgSF (Ig Superfamily) of cell Adhesion molecules (CAM) expressed on endothelial cells, for example, antibodies or fragment thereof which bind to ICAM-1, ICAM-2, ICAM-3, VCAM-1, MAdCAM-1, E-cadherin, JAM-1, JAM-2, JAM-3. In alternative embodiments, a targeting moiety can be an integrin or fragment or variant or homologue thereof expressed by leukocyte that binds to an integrin ligand. Examples of such integrins useful as targeting moieties for a endothelial delivery agent as disclosed herein can be, for example but not limited to; LFA-1 (αLβ2), Mac-1 (αMβ2), p150.95 (αXβ2), αDβ2, VLA-4 (α4β1), and β₇ (α4β7 and αEβ7) or fragments, homologues or variants thereof. An endothelial delivery agent as disclosed herein is useful to delivering agents to endothelial cells contributing to a pathogenesis, such as abnormal or aberrant angiogenesis and/or inflamed endothelial cells.

One aspect of the present invention provides a leukocyte-selective delivery agent comprising, a targeting moiety that selectively binds one or more integrins on the surface of a leukocyte, wherein the integrin is in an active conformation; a carrier particle associated with the targeting moiety, wherein the carrier particle having a lipid phase and an aqueous phase; an agent entrapped in the lipid phase of the carrier particle; and/or an agent entrapped in the aqueous phase of the carrier particle.

In some embodiments, present invention provides a leukocyte-selective delivery agent comprising, a targeting moiety that selectively binds one or more integrins on the surface of a leukocyte; a carrier particle associated with the targeting moiety, wherein the carrier particle having a lipid phase and an aqueous phase; an agent entrapped in the lipid phase of the carrier particle; and/or an agent entrapped in the aqueous phase of the carrier particle.

In some embodiments, a leukocyte-selective delivery agent as disclosed herein is further selective for activated leukocytes, and in some embodiments, a targeting moiety selectively binds to leukocyte specific integrins their activated conformation. In some embodiments, integrin is selected from the group consisting of LFA-1 (αLβ2), Mac-1 (αMβ2), p150.95 (αXβ2), αDβ2, VLA-4 (α4β1), and β₇ (α4β7 and αEβ7), or derivatives or homologues thereof. In some embodiments, the integrins are human integrins.

In some embodiments, leukocyte-selective delivery agent as disclosed herein comprises integrins that can bind an integrin ligand selected from the group consisting of ICAM-1, ICAM-2, ICAM-3, VCAM-1, MAdCAM-1, E-cadherin, JAM-1, JAM-2 and JAM-3.

An example of an embodiment of the leukocyte-selective delivery agent as disclosed herein comprises an integrin such as LFA-1 and a targeting moiety which comprises an antibody or functional fragment thereof, where the targeting moiety binds to the locked open I domain of LFA-1, or binds to the leg domain of the β₂ subunit of LFA-1 (α_{L}β₂) or integrin β₇.

In some embodiments, a targeting moiety useful in the leukocyte-selective delivery agents as disclosed herein is an antibody or integrin ligand, or functional fragments or variants thereof, for example but not limited to a scFv, an IgG, Fab′, F(ab′)₂, or a recombinant bivalent scFv, or fragments thereof. In some embodiments, the targeting moiety comprises an antibody or functional fragment thereof, which binds non-selectively to low affinity and high affinity LFA-1, Mac-1 and integrin β₇.

In some embodiments, a carrier particle useful in the leukocyte-selective delivery agent as disclosed herein comprises a liposome or other lipid or non-lipid carrier or a functional fragment thereof. In some embodiments, the liposome is unilamellar with a first layer comprising glycosaminoglycan hyaluronan (HA) covalently linked to phosphatidylethanolamine therein, and a second layer comprising specific antibodies covalently attached to the HA of the first layer.

In some embodiments, the leukocyte-selective delivery agent as disclosed herein comprises one or more agents selected from the group consisting of an RNA interference (RNAi) molecule, a small molecule, a polypeptide, lipophilic agent, hydrophobic agent, antibody or analogues, variants or functional fragments thereof, including for example, but not limited to RNA interference molecules such as siRNA, dsRNA, stRNA, shRNA, miRNA, and combinations thereof. In some embodiments, such an RNAi molecule functions in gene silencing of CCR5, ku70, CD4 or cyclin-D1, or derivatives or homologues thereof. In some embodiments, the leukocyte-selective delivery agent as disclosed herein comprises a hydrophobic agent, for example but not limited to hydrophobic agents such as paclitaxel, platinum-based drugs, anthracyclines, mitomycin C, and compounds of the quinolone family of synthetic antibacterial compounds, such as for example enoxacin, ciprofloxacin, ofloxacin, norfloxacin, and difloxacin and combinations and analogues thereof. In addition, the leukocyte-selective delivery agent as disclosed herein comprises a hydrophobic agent such as a lipophilic RNAi or an insoluble RNAi agent, whereby the RNAi or siRNA agent has been modified to become insoluble by addition of cholesterol, by methods commonly known by persons of ordinary skill in the art.

Another aspect of the present invention provides methods for delivery of an agent to a leukocyte comprising administering to a biological sample comprising leukocytes, a leukocyte-selective delivery agent as disclosed herein, wherein the leukocyte-selective delivery agent comprises a targeting moiety that selectively binds one or more integrins on the surface of a leukocyte, wherein the integrin in an activated conformation; a carrier particle associated with the targeting moiety, wherein the carrier particle has a lipid phase and a aqueous phase; wherein a lipophilic agent or hydrophobic agent is associated with the lipid phase of the carrier particle and/or a hydrophilic agent is associated with the aqueous phase of the carrier particle, and contacting the leukocyte delivery agent with a leukocyte, wherein contacting the leukocyte delivery agent with the leukocyte delivers the agents to the leukocyte. In some embodiments, the leukocyte being delivered an agent by the leukocyte-selective delivery agent is an activated leukocyte. In some embodiments, the biological sample comprising leukocytes is present in a subject. In alternative embodiments, a biological sample is obtained from a subject.

In some embodiments, the method to deliver agents to leukocytes further comprising administering the leukocytes that have been contacted with a leukocyte delivery agent comprising one or more agents to a subject, wherein the leukocytes have had agents delivered by the leukocyte-selective delivery agent. In some embodiments, the methods as disclosed herein are useful to deliver agents to leukocytes to a biological samples that are ex vivo, or in vivo or in vitro biological samples. In some embodiments, the biological sample is human and in some embodiments the methods are useful to deliver agents to human leukocytes, and in some embodiments a subject is a human, for example but not limited to a subject with inappropriate leukocyte activation. In some embodiments, a leukocyte being delivered agents by the methods and compositions as disclosed herein has inappropriate leukocyte activation.

In some embodiments, an integrin for use in the methods as disclosed herein or a leukocyte delivery agent comprises LFA-1 and also comprises a targeting moiety which is an antibody, or functional fragment thereof which binds to LFA-1 in a locked open I domain configuration with higher affinity as compared to LFA-1 in a locked closed I domain configuration, or the targeting moiety binds to the leg domain of the β₂ subunit of LFA-1 (α_{L}β₂). In some alternative embodiments, integrin for use in the methods as disclosed herein or a leukocyte delivery agent comprises the integrin is β₇ and the targeting moiety comprises an antibody or functional fragment thereof, which binds to integrin β₇.

Another aspect of the present invention relates to a method for delivery of agents to a leukocyte present in a subject, comprising: administering to a subject leukocyte-selective delivery agent as disclosed herein, wherein the leukocyte-selective delivery agent comprises: a targeting moiety that selectively binds one or more integrins on the surface of a leukocyte, wherein the integrin in an activated conformation; a carrier particle associated with the targeting moiety, wherein the carrier particle has a lipid phase and a aqueous phase; wherein a lipophilic agent or hydrophobic agent is associated with the lipid phase of the carrier particle and/or a hydrophilic agent is associated with the aqueous phase of the carrier particle, and the method involves contacting the leukocyte delivery agent with a leukocyte, wherein contacting the leukocyte delivery agent with the leukocyte delivers the agents to the leukocyte. In some embodiments, the method comprises a leukocyte-selective delivery agent is further selective for activated leukocytes.

In some embodiments, the method comprises a leukocyte-selective delivery agent which comprises a targeting moiety which binds with higher affinity to integrins in an activated conformation as compared to integrins in an inactive conformation. In some embodiments, the method comprises a leukocyte-selective delivery agent which comprises an integrin selected from the group consisting of LFA-1, Mac-1, and β₇. For example but not limited to, the method comprises use of a leukocyte-selective delivery agent which comprising a the LFA-1 integrin and the targeting moiety comprises an antibody or functional fragment thereof, wherein the targeting moiety binds to LFA-1 in a locked open I domain configuration with higher affinity as compared to LFA-1 in a locked closed 1 domain configuration, or the targeting moiety binds to the leg domain of the β₂ subunit of LFA-1 (α_{L}β₂). In some embodiments, an integrin useful in the methods using a leukocyte-selective delivery agent is β₇ and the targeting moiety comprises an antibody or functional fragment thereof, which binds to the β₇ integrin.

In particular embodiments, a targeting moiety comprises an antibody or functional fragment thereof, for example but not limited to scFv, IgG, Fab′, F(ab′)₂, and a recombinant bivalent scFv. In some embodiments, a leukocyte-selective delivery agent comprises a targeting moiety which can be an antibody or functional fragment thereof, which binds non-selectively to both low affinity and high affinity LFA-1 and to Integrin β₇.

In further embodiments, a carrier particle of a leukocyte-selective delivery agent comprises a liposome or a lipid particle or a non-lipid particle and a functional fragment thereof, for example but not limited to liposome which can be unilamellar, for example with a first layer comprising glycosaminoglycan hyaluronan (HA) covalently linked to phosphatidylethanolamine therein, and a second layer comprising specific antibodies covalently attached to the HA of the first layer.

In the methods and the leukocyte-selective delivery agent as disclosed herein comprise at least one agent, for example but not limited to agents selected from the group consisting of an RNA interference (RNAi) molecule, a small molecule, a polypeptide, a hydrophobic agent, a poorly soluble drug and an antibody or functional fragment thereof, for example RNA interference molecule is selected from the group consisting of siRNA, dsRNA, stRNA, shRNA, miRNA, and combinations thereof. In particular embodiments, an agent for use in the methods of the present invention can comprise a siRNA such as, for example, CCR5-siRNA, ku70-siRNA, CD4-siRNA or cyclin-D1-siRNA.

In some embodiments, am agent is a hydrophobic agent, for example but not limited to hydrophobic molecules such as paclitaxel, platinum-based drugs, anthracyclines, mitomycin C and compounds of the quinolone family of synthetic antibacterial compounds, such as for example enoxacin, ciprofloxacin, ofloxacin, norfloxacin, and difloxacin and combinations and analogues thereof. In another embodiment, a hydrophobic agent can be a lipophilic RNAi or an insoluble RNAi agent, whereby the RNAi or siRNA agent has been modified to become insoluble by addition of cholesterol, by methods commonly known by persons of ordinary skill in the art, such a cationic lipopeptide (Unnamalai, et al (2004). FEBS J. 566: 307-310, steroid and lipid conjugates of siRNA (Lorenz, et al., C., (2004). Bioorg. Med. Chem. Lett. 14: 4975-4977; Spagnou, et al., (2004). Biochemistry 43: 13348-13356) which are lipophilic siRNAs conjugated with derivatives of cholesterol, lithocholic acid or lauric acid (Wolfrum et al., Nat Biotechnol. 2007 October; 25(10):1149-57).

Another aspect of the present invention relates to a system for delivering an agent to a leukocyte, the system comprising; a targeting moiety that selectively binds one or more integrins on the surface of a leukocyte; a carrier particle associated with the targeting moiety, wherein the carrier particle having a lipid phase and an aqueous phase; wherein a lipophilic agent or hydrophobic agent can be entrapped in the lipid phase of the carrier particle and/or a hydrophilic agent can be entrapped in the aqueous phase of the carrier particle. In such embodiments, the integrin is selected from the group consisting of LFA-1 (αLβ2), Mac-1 (αMβ2), p150.95 (αXβ2), αDβ2, VLA-4 (α4β1), and β₇ (α4β7 and αEβ7), and the integrin can bind an integrin ligand selected from the group consisting of ICAM-1, ICAM-2, ICAM-3, VCAM-1, MAdCAM-1, E-Cadherin, JAM-1, JAM-2 and JAM-3.

Another aspect of the present invention provides an endothelial cell-selective delivery agent comprising; a targeting moiety that selectively binds one or more integrin ligands on the surface of a endothelial cell; a carrier particle associated with the targeting moiety, wherein the carrier particle having a lipid phase and an aqueous phase; an agent entrapped in the lipid phase of the carrier particle; and/or an agent entrapped in the aqueous phase of the carrier particle.

In some embodiments, integrin ligand useful in endothelial cell-selective delivery agents can be selected from the group consisting of ICAM-1, ICAM-2, ICAM-3, VCAM-1, MAdCAM-1, E-Cadherin, JAM-1, JAM-2 and JAM-3, or other integrin ligands and/or molecules that can bind to an integrin present on the surface of leukocytes. Such integrin ligands and/or molecules can bind to an integrin selected from the group consisting of LFA-1 (αLβ2), Mac-1 (αMβ2), p150.95 (αXβ2), VLA-4 (α4β1), and 137 (α4β7 and αEβ7).

In some embodiments, the targeting moiety of an endothelial cell-selective delivery agent can be an antibody or integrin, or functional fragments or variants thereof, for example a scFv, an IgG, Fab′, F(ab′)₂, or a recombinant bivalent scFv, or fragments thereof.

In some embodiments, a carrier particle of an endothelial cell-selective delivery agent can be a liposome or other lipid or non-lipid carrier or a functional fragment thereof, for example a liposome that is unilamellar, with a first layer comprising glycosaminoglycan hyaluronan (HA) covalently linked to phosphatidylethanolamine therein, and a second layer comprising specific antibodies covalently attached to the HA of the first layer.

In some embodiments, the endothelial cell-selective delivery agent as disclosed herein can deliver agent, such as but not limited to RNA interference (RNAi) molecules, a small molecule, a polypeptide, lipophilic agent, hydrophobic agent, antibody or analogues, variants or functional fragments thereof. In some embodiments, a RNA interference molecule can be, for example but not limited siRNA, dsRNA, stRNA, shRNA, miRNA, and combinations thereof. In particular embodiments, such a RNAi molecule can function in gene silencing VEGF, and/or other angiogenesis genes, which are commonly known by persons of ordinary skill in the art.

In some embodiments, the endothelial cell-selective delivery agent as disclosed herein can deliver hydrophobic agents, for example but not limited to paclitaxel, platinum-based drugs, anthracyclines, mitomycin C and compounds of the quinolone family of synthetic antibacterial compounds, such as for example enoxacin, ciprofloxacin, ofloxacin, norfloxacin, and difloxacin and combinations and analogues thereof.

Another aspect of the present invention relates to a method for delivery of an agent to an endothelial cell comprising: administering to endothelial cells an endothelial cell-selective delivery agent as disclosed herein, wherein the leukocyte-selective delivery agent comprises; a targeting moiety that selectively binds one or more integrin ligands on the surface of an endothelial cell; a carrier particle associated with the targeting moiety, wherein the carrier particle has a lipid phase and a aqueous phase; wherein a lipophilic agent or hydrophobic agent is associated with the lipid phase of the carrier particle and/or a hydrophilic agent is associated with the aqueous phase of the carrier particle, and where the endothelial cell-selective delivery agent is contacted with an endothelial cell to deliver the agents to the endothelial cell. In some embodiments, administration is to a subject or a biological sample, for example a biological sample is obtained from a subject, or an ex vivo, or in vitro biological sample. In some embodiments, methods of delivery of an agent to an endothelial cell comprises further comprises the steps of administering endothelial cells which have been delivered an agent following their contact with the endothelial cell-selective delivery agent to a subject. In some embodiments, the method for delivery of an agent to an endothelial cell encompasses delivery to endothelial cells in a subject, for example a human, and in particular embodiments, the subject has inappropriate endothelial cell proliferation. In some embodiments, the subject is a human. In some embodiments, the endothelial cell has inappropriate endothelial cell proliferation.

Another aspect of the present invention provides a system for delivering an agent to an endothelial cell, the system comprising; a targeting moiety that selectively binds one or more integrin ligands on the surface of an endothelial cell; a carrier particle associated with the targeting moiety, wherein the carrier particle having a lipid phase and an aqueous phase; wherein a lipophilic agent or hydrophobic agent can be entrapped in the lipid phase of the carrier particle and/or a hydrophilic agent can be entrapped in the aqueous phase of the carrier particle.

In some embodiments, integrin ligand useful in such as system can be selected from the group consisting of ICAM-1, ICAM-2, ICAM-3, VCAM-1, MAdCAM-1, E-Cadherin, JAM-1, JAM-2 and JAM-3, or other integrin ligands and/or molecules that can bind to an integrin present on the surface of leukocytes. Such integrin ligands and/or molecules can bind to an integrin selected from the group consisting of LFA-1 (αLβ2), Mac-1 (αMβ2), p150.95 (αXβ2), αDβ2, VLA-4 (α4β1), and β₇ (α4β7 and αEβ7).

In some embodiments, the targeting moiety of an endothelial cell-selective delivery agent useful in such a system can be an antibody or integrin, or functional fragments or variants thereof, for example a scFv, an IgG, Fab′, F(ab′)₂, or a recombinant bivalent scFv, or fragments thereof.

In some embodiments, a carrier particle of an endothelial cell-selective delivery agent useful in such a system can be a liposome or other lipid or non-lipid carrier or a functional fragment thereof, for example a liposome that is unilamellar, with a first layer comprising glycosaminoglycan hyaluronan (HA) covalently linked to phosphatidylethanolamine therein, and a second layer comprising specific antibodies covalently attached to the HA of the first layer.

Another aspect of the present invention relates to use of the compositions as disclosed herein comprising a carrier particle (comprising both an insoluble agent and a soluble agent) associated with a targeting moiety to deliver the insoluble agent and soluble agent to selected a target cell. In some embodiments, the insoluble agent and soluble agent have synergistic or additive effects. As an illustrative example only, a leukocyte delivery agent or endothelial cell delivery agent can be used to deliver two agents which function by two independent mechanisms or cellular pathways for a common outcome. For example and as disclosed herein and in the Examples, the inventors demonstrate the use of a leukocyte delivery agent to deliver a soluble anti-cancer agent and an insoluble anti-cancer agent to kill an immortalized cancer cell line using separate biological cell death pathways. In Example 7, the inventors demonstrate use of a leukocyte delivery agent to deliver an insoluble agent (i) a siRNA to decrease the expression of CyD1 which functions to inhibit the continuation of the cell cycle, and (ii) TAXOL® which inhibits cell cycle progression by interfering with the mechanisms which are necessary for dividing cells. Thus, Example 7 demonstrates the delivery of two agents; a soluble agent and an insoluble agent which function by different mechanisms to inhibit cell cycle progression. By way of another example, one use the compositions as disclosed herein for antivirus small molecule therapy in the treatment of a subject with a disease caused by a virus. For example, one deliver a soluble agent such as an anti-HIV siRNA in combination with an insoluble anti-HIV agent, such as for example the reverse-transcriptase inhibitor AZT/azidothymidine, where the effect of both the insoluble agent (the anti-HIV RNAi) and soluble agent (AZT) are additive to one another as they function by different mechanisms and different pathways to inhibit HIV viral replication, thus are additive to each other with respect to they both function to inhibit HIV viral replication by independent biological pathways. In some embodiments, useful anti-HIV siRNA molecules which can be used in combination with an insoluble anti-HIV agent include, for example, but are not limited to si-CD4, si-CCR5, si-HIVgag, si-Vif, si-Tat and modified siRNA variants which gene silence at least one gene selected from CD4, CCR5, HIVgag, Vif or Tat or variants thereof.

Accordingly, as demonstrated herein, the compositions as disclosed herein can be used to for dual delivery of agents which function by two independent mechanisms for the same biological outcome. Stated another way, the compositions as disclosed herein can be used to for dual delivery of at least one insoluble agent and at least one soluble agent which have additive effects by two independent mechanisms for the same biological outcome. The term “additive” as used herein refers to refers to an increase in effectiveness of a first agent in the presence of a second agent as compared to the use of the first agent alone. Without wishing to be bound to theory, a soluble agent (such as an anti-HIV RNAi) and an insoluble agent (such as AZT) which function by different mechanisms and on different cell pathways will typically function as additive agents.

In another embodiment, the compositions as disclosed herein can be used to for dual delivery of agents which function synergistically together. The term ‘synergistically” or “synergy” or “synergistic” as used herein refers to the interaction of two or more agents so that their combined effect is greater than each of their individual effects at the same dose alone. Without wishing to be bound to theory, a soluble agent (e.g. a RNAi to CycD1) and an insoluble agent (such as TAXOL®) which function by different mechanisms but on the same cellular pathway for a common biological outcome will typically function as synergistic agents.

### BRIEF DESCRIPTION OF FIGURES

FIGS. 1A-1G show binding, delivery of siRNAs and internalization of integrin 137-targeted stabilized nanoparticles β₇ It-sNP) into variety of cells. FIG. 1A shows the delivery and binding of integrin β₇-targeted stabilized nanoparticles β₇ It-sNP) to mouse spleenocytes. The dotted line indicates the absence of β₇ It-sNP, the solid line represents the β₇ expression in the presence of β₇ It-sNP. FIG. 1B shows Cy3-siRNAs or Ku70-siRNAs were entrapped in β₇ It-sNP and delivered to mouse primary splenocytes, and FIG. 1C shows delivery to mouse T cells (TK-1 cells). FIG. 1D shows knockdown of Ku70 expression in splenocytes [1d(1)] and TK-1 cells [1d(2)]. FIG. 1D also shows a graph of cell number and Ku70 expression, and a graph of cell number and Ku70 expression, where β₇ It-sNP with Ku70-siRNA (black solid line) or Luci-siRNA (thin solid line) or Ig-sNP with Ku70-siRNA (dotted line) are shown in the left panel. FIG. 1E shows binding and knockdown of Ku70 expression in human PBMC (left panel and right panel), where β₇ It-sNP carry Ku-70-siRNA (black solid line) or Cy3-siRNA (thin solid line) or no siRNA (dotted line). FIG. 1F shows that no interferon-responsive genes, such as STAT1, OAS1 or INF-β are activated when siRNAs are entrapped in either isotype control nanoparticles (Ig-sNP) or in β₇ It-sNP. FIG. 1G shows no lymphocyte activation when siRNAs are entrapped in either Isotype control nanoparticles (Ig-sNP) or in β₇ It-sNP. (It-sNP=integrin-targetted, stabilized nanoparticle).

FIGS. 2A-2C show in vivo silencing of Ku70 delivered via β₇ It-sNP to gut-associated tissues and pharmacokinetics and organ tissue distribution profile of Ig-sNP and β₇ It-sNP injected systemically to healthy mice. FIG. 2A shows in vivo silencing of Ku70 delivered via β₇ It-sNP to gut-associated tissues in β7 WT (wild type) mice. FIG. 2B shows the pharmacokinetics of Ig-sNP and β₇ It-sNP injected systemically to healthy mice, showing that β7-IT-sNP remains in the blood for at least 10 hours, and remains at a higher level than Ig-sNP. FIG. 2C shows distribution profile of Ig-sNP and β₇ It-sNP in different organs after systemic injections into healthy (WT) mice.

FIGS. 3A-3C show in vitro and in vivo silencing of Cyclin-D1 (CD1), a cell cycle regulator. FIG. 3A shows real time RT-PCR which was performed on mouse primary splenocytes, showing mRNA levels of Cyclin D1 are decreased when CD1-siRNAs are delivered through β₇ It-sNP, normalized to the housekeeping gene GAPDH. FIG. 3B shows decreased proliferation (³H-Thymidine incorporation assay) in primary splenocytes treated with β₇ It-sNP entrapping CD1-siRNAs. FIG. 3C shows a single intravenous injection of β₇ It-sNP entrapping CD1-siRNAs (2.5 mg/Kg body) or control formulations (same siRNAs conc.) showed decrease intrinsic proliferation in cells expressing integrin β₇ assayed by ³H-Thymidine incorporation 2 days post injection.

FIGS. 4A-4F show CD1-siRNAs delivered by β₇ It-sNP selectively reduces inflammation in an experimental colitis model. FIG. 4A shows the pharmacokinetics of Ig-sNP and β₇ It-sNP in an experimental colitis induced by DSS. FIG. 4B shows the tissue profile distribution of Ig-sNP and β₇ It-sNP in an experimental colitis induced by DSS. FIG. 4C shows the bodyweight changes in mice with experimental colitis, treated with different formulations CD1-siRNAs delivered via Ig-sNP or β₇ It-sNP. Arrows in FIG. 4C indicate days of intravenous administration of these formulations. FIG. 4D shows representative images from histology (H & E staining) of colon sections taken from healthy mice or mice with experimental colitis treated with CD1-siRNAs entrapped in Ig-sNP or β₇ It-sNP. FIG. 4E shows the hematocrit levels of healthy or diseased mice with experimental colitis treated with targeted system entrapping CD1-siRNAs or control siRNA. FIG. 4F shows quantitative RT-PCR of 3 genes (CD1, TNF-α, and IL-12p40) in response to treatment with CD1-siRNAs delivered via different formulations, normalized to mRNA of GAPDH in mice with colitis.

FIGS. 5A-5C show binding, internalization and delivery of siRNAs in LFA-1 It-sNP. FIG. 5A shows the binding of TS1/22 (mouse anti-human integrin LFA-1)-It-sNP (LFA-1 It-sNP) to K562 cells expressing-integrin LFA-1. FIG. 5B shows confocal image shows binding and siRNA delivery via LFA-1 It-sNP to K562 cells expressing-integrin LFA-1. FIG. 5C shows Cy3-siRNA uptake to cells expressing K562 LFA-1 via LFA-1 I-tsNP as compared to K562 cells not expressing LFA-1 (parent cells). (It-sNP=integrin-targetted, stabilized nanoparticle)

FIG. 6 shows in vitro cell survival upon treatment with paclitaxel entrapped in LFA-1 It-sNP for short exposure time (4 h) in k562 LFA-1 and parent cells. (It-sNP=integrin-targetted, stabilized nanoparticle)

FIG. 7 shows in vitro combinational treatment with paclitaxel and CD1-siRNAs entrapped in LFA-1 It-sNP dramatically reduce cell viability in K562 cells-expressing integrin LFA-1.

FIGS. 8A-8G shows a schematic illustration of the steps required for generating I/IL-tsNP. FIG. 8A shows a multilamellar vesicle (MLV) as prepared as discussed in the method section. FIG. 8B shows MLV is extruded to form a unilamellar vesicle (ULV) with a diameter of ˜100 nm. FIG. 8C shows the coating with high molecular weight hyaluronan provides a stabilizing layer protecting the nano-dimensions in the lyophilization process later on. FIG. 8D shows an additional coating with an antibody for the targeting delivery to a target cells, (i.e. coating with antibodies against integrins or an integrin ligand for guiding delivery of the entrapped agents to cells expressing the this integrin. FIG. 8E shows Lyophilization, necessary for the entrapment of drugs and for long self-life. FIG. 8F shows pre-condensing siRNAs with a cationic protein (protamine) to neutralize the negative charge from the siRNAs and rehydration the lyophilized sNP to entrap the siRNAs. FIG. 8G shows I/IL-tsNP entrapping siRNAs. Ultracentrifugation prior to use can remove unentrapped siRNAs. (I/IL-tsNP=integrin/integrin-ligand targetted stabilized nanoparticle).

FIG. 9 shows a schematic illustration of the encapsulation of poorly soluble (lipophilic or hydrophobic) and soluble (hydrophilic) drugs in a I/IL-tsNP. FIG. 9A shows a multilamellar vesicle (MLV) as prepared as discussed in the method section and entrapping a insoluble drug (for example Taxol). FIG. 9B shows MLV is extruded to form a unilamellar vesicle (ULV) with a diameter of ˜100 nm. FIG. 9C shows the coating with high molecular weight hyaluronan provides a stabilizing layer protecting the nano-dimensions in the lyophilization process later on. FIG. 9D shows an additional coating with an antibody for the targeting delivery to a target cells (i.e. coating with antibodies against integrins or an integrin ligand for guiding delivery of the entrapped agents to cells expressing this integrin). FIG. 9E shows lyophilization, necessary for the entrapment of drugs and for long self-life. FIG. 9F shows pre-condensing with a soluble agent and rehydration the lyophilized sNP to entrap the siRNAs. FIG. 9G shows I/IL-tsNP entrapping simultaneously both a lipophilic (hydrophobic) agent and a soluble (hydrophilic) agent. Ultracentrifugation prior to use can remove unentrapped soluble agents. (I/IL-tsNP=integrin/integrin-ligand targetted stabilized nanoparticle).

FIGS. 10A-10C show simultaneous delivery of a soluble agent (CyD1 RNAi) and an insoluble agent (Taxol) to K562s transfected cells with LFA-1. FIG. 10A shows silencing by siRNA-CyclinD1 in just a nanoparticle-siRNA system. In FIG. 10A, CyD1-siRNA in α_{L}, I-tsNP induced dose-dependent silencing in K562 cells expressing LFA-1. CyD1 expression was examined by flow cytometry. FIG. 10B shows the diameter and zeta potential measurement of α_{L} I-tsNP-Taxol before and after siRNA entrapment. All measurements were performed using a Zetasizer nano ZS instrument (Malvern) at pH 6.7, 10 mM NaCl at 20° C. FIG. 10C shows the synergistic cyotoxic effects by CyD1-siRNA (CyD-siRNA) and paclitaxel (Taxol) co-formulated in α_{L} I-tsNP (I-tsNP). K562 cells expressing LFA-1 were treated as indicated for 72 h, and viability was determined by MTT assay. Data are expressed as the mean±SEM from three independent experiments. P<0.01 vs mock-treated*, and paclitaxel-formulating αL I-tsNP-treated** cells. (I-tsNP=integrin targetted stabilized nanoparticle)

### DETAILED DESCRIPTION OF THE INVENTION

The present invention relates to methods and compositions for the simultaneous delivery of at least one insoluble agent and at least one soluble agent to a cell. In some embodiments, the inventors have discovered methods and compositions for the delivery of an insoluble agent and a soluble agent to a particular target cell, for example, a target cell can be a leukocyte. In particular, the inventors have discovered a method to deliver at least one agent, for example a hydrophilic agent and/or a hydrophobic agent to leukocytes. In some embodiments, the inventors have discovered a method to simultaneously deliver a hydrophilic (i.e. soluble) agent and/or a hydrophobic (i.e. insoluble) agent to a leukocyte cell.

The inventors have discovered a method to deliver agents to leukocytes by associating a targeting moiety to a carrier particle, where the targeting moiety has affinity for, or binds to integrins present on the surface of leukocytes, and where an agent is associated with the carrier particle. Accordingly, the present invention relates to a leukocyte delivery agent. A “leukocyte delivery agent” as disclosed herein, comprises a targeting moiety that has affinity for integrins on leukocytes, where the targeting moiety is associated with a carrier particle.

The role of integrins in leukocyte-endothelial cell interactions plays an important role in leukocyte invasion at a tissue injury site. Integrins are useful as cell surface target markers to be targeted for the delivery of agents to leukocytes for the following reasons:

Firstly, 18 α-subunits and 8 β-subunits of integrins have been identified thus far, forming at least 24 different integrin heteromiders that are expressed in various types of cells. β2 integrins (α_{L}β₂, α_{M}β₂, α_{X}β₂, α_{D}β₂) and β₇ integrins (α₄β₇, and α_{E}β₇) are exclusively expressed on leukocytes. In adults, α₄β₁ is also exclusively expressed on leukocytes. Thus, one example of an integrin which could be targeted by a targeting moiety is integrin LFA-1 (α_{L}β2), as disclosed herein and in the Examples. Stated another way, a targeting moiety which has affinity for and binds specifically to an integrin which is exclusively expressed on leukocyte cells, such as LFA-1 is useful to deliver the carrier particle comprising the insoluble and/or soluble particles to leukocyte cells. This unique expression of LFA-1 to leukocytes makes this integrin useful for leukocyte-specific targeting.

Secondly, integrins are constitutively internalized and recycled in leukocytes. Regulated internalization of integrins on leukocytes is implicated in facilitating detachment for efficient directional cell migration (Fabbri et al., 2005) as well as phagocytosis. Ligand-derived peptides (Anderson and Siahaan, 2003) as well as antibodies to integrins on leukocytes (Coffey et al., 2004) have been shown to induce internalization. Thus, integrin recycling supports internalization of bound antibodies and peptides, as well as their carrier particles, a requisite for efficient intracellular drug delivery.

Thirdly, integrins are useful as highly specific target markers for activated leukocytes as they convert into a high-affinity conformation (which exposes distinct epitopes). The inventors have previously recently demonstrated using siRNA delivery directed by an engineered monoclonal antibody (mAB) AL-57 that selectively binds to the high-affinity conformation of α_{L}β₂ integrin (LFA-1) (Peer et al., 2007). As LFA-1-mediated internalization and lysosomal degradation are proposed to be a major pathway to clear LFA-1 antibodies from circulation (Coffey et al., 2004), the selective targeting to the active LFA-1 would improve pharmacokinetics by eliminating unnecessary mAb binding. In some embodiments, selective targeting of the activated and adhesive leukocytes would be useful for suppressing inflammatory tissue injury caused by leukocyte accumulation. Furthermore, by leaving naïve cells untouched, selective targeting would be advantageous in reducing iatrogenic immune-defects (i.e. diseases or disorders inadvertently induced by a physician or surgeon or by medical treatment or a diagnostic procedure).

Fourthly, using targeting moieties which function to bind to a cell surface marker on the target cell, as well also block the function of the cell surface marker is expected to produce additive or synergistic effects of silencing of pro-inflammatory molecules. For example, a leukocyte targeting moiety can serve a dual function; (i) it can binds to LFA-1 to deliver the carrier particle comprising insoluble and soluble agents to the leukocyte, and (ii) inhibit LFA function to inhibit LFA-1-mediated cell adhesion. The blocking single integrin alone is not sufficient to suppress inflammation in certain disease models (de Fougerolles, 2003). The inventors have discovered that the combination of targeting moieties which function as blocking antibodies, combined with gene silencing (mediated by soluble agents comprised within the carrier particle) is a novel therapeutic approach to overcome the existing limitations of blocking one integrin alone. For instance, a cell surface integrin can be blocked by a leukocyte targeting moiety, which is conjugated to a carrier particle comprising an RNAi inhibitory agent specific to a different cell surface integrin. As an example only, the RNAi agent can target the knockdown and inhibition of the integrin β₇. Integrin β₇ plays an important role in lymphocytes trafficking to the gut by associating with two other integrins (a chains) to form α₄β₇, which is a homing receptor of lymphocytes to gut-associate tissues and α_{E}β₇, which involves in adhesion of lymphocytes to the intestinal epithelium (Luster et al., 2005). β₇ integrin-mediated migration of T lymphocytes is implicated in the pathogenesis of intestinal inflammation (Feagan et al., 2005; Sydora et al., 2002).

Accordingly, one aspect of the present invention relates to methods and compositions for the delivery of an insoluble and/or soluble agent to a leukocyte, wherein the composition comprises a leukocyte targeting moiety conjugated to a carrier particle comprising an insoluble and soluble agent. In such an embodiment, one can use any targeting moiety which has affinity for, or binds to integrin expressed in leukocytes, for example but not limited to of LFA-1 (αLβ2), Mac-1 (αMβ2), p150.95 (αXβ2), αDβ2, VLA-4 (α4β1), and β₇ (α4β7 and αEβ7) are useful as targeting moieties as disclosed herein. In some embodiments, such a targeting moiety is an antibody or fragment thereof with affinity for, or binds LFA-1 (αLβ2), Mac-1 (αMβ2), p150.95 (αXβ2), αDβ2, VLA-4 (α4β1), and β₇ (α4β7 and αEβ7). In alternative embodiments, a targeting moiety can be an integrin ligand or fragment or variant or homologue thereof that binds to integrins; LFA-1 (αLβ2), Mac-1 (αMβ2), p150.95 (αXβ2), αDβ2, VLA-4 (α4β1), and β₇ (α4β7 and αEβ7). Examples of such integrin ligands useful as targeting moieties of the present invention include, for example but are not limited to members of the IgSF (Ig Superfamily) of cell Adhesion molecules (CAM) expressed on endothelial cells, for example, ICAM-1, ICAM-2, ICAM-3, VCAM-1, MAdCAM-1, E-cadherin, JAM-1, JAM-2, JAM-3 or fragments, homologues or variants thereof.

The present invention relates to methods and compositions for the delivery of an insoluble and/or soluble agent to a leukocyte in vitro or in vivo using a composition which comprises a leukocyte targeting moiety conjugated to a carrier particle comprising an insoluble and soluble agent. Such a composition is referred to herein and throughout the application as a “leukocyte delivery agent”. Leukocyte delivery agents as referred to herein and in the Examples is also sometimes referred to as an “integrin-targeted and stabilized nanoparticles” or “I-tsNP” or “It-sNP”, where the leukocyte targeting moiety component of the leukocyte delivery agent has affinity for, or binds to integrins present on the leukocytes. The inventors demonstrate, using the leukocyte delivery agent as disclosed herein, for the efficient delivery of a hydrophilic (soluble) agent, such as cyclin D1-siRNA to leukocytes, as detected by potent gene silencing to suppress aberrant cellular proliferation and inflammatory tissue damage in an animal model of colitis.

The inventors also demonstrate simultaneous delivery of a hydrophobic and a hydrophilic agent to leukocytes using the leukocyte delivery agent as disclosed herein. As disclosed in Example 7, using a I-tsNP the inventors demonstrate delivery of a hydrophilic agent, for example cyclin D1 siRNAs and a hydrophobic agent, such as Paclitaxel to cancer cells, therefore demonstrating delivery of agents with different biological functions, as both cyclin D1 siRNAs and Paclitaxel interfere with distinct steps in cell-cycle progression to producing synergistic anti-proliferative (anti-cancer) effects.

Accordingly, another aspect of the present invention relates to the delivery of at least one agent, and in some embodiments, more than one agent to leukocytes.

Another aspect of the present invention relates to the use of the leukocyte delivery agent to deliver agents to leukocytes for the treatment and prevention of a wide range of inflammatory, degenerative, and malignant diseases.

Alternatively, the present invention relates to methods and compositions for the delivery of an insoluble and/or soluble agent to an endothelial cell in vitro or in vivo using a composition which comprises an endothelial cell targeting moiety conjugated to a carrier particle comprising an insoluble and soluble agent. Such a composition is referred to herein and throughout the application as a “endothelial cell delivery agent”. Endothelial cell delivery agents as referred to herein and in the Examples is also sometimes referred to as an “integrin ligand-targeted and stabilized nanoparticles” or “IL-tsNP” or “ILt-sNP”, where the endothelial cell targeting moiety component of the endothelial delivery agent has affinity for, or binds to integrin ligands present on the endothelial cells.

Another aspect of the present invention relates to the use of an endothelial delivery agent to deliver agents to endothelial cells, where a “endothelial delivery agent” comprises a targeting moiety that has affinity for integrin ligands on endothelial cells, where the targeting moiety is associated with a carrier particle. In such embodiments, such a endothelial targeting moiety that has affinity for, or binds to integrin ligands expressed on endothelial cells, for example but not limited to members of the IgSF (Ig Superfamily) of cell Adhesion molecules (CAM) expressed on endothelial cells, for example, antibodies or fragment thereof which bind to ICAM-1, ICAM-3, VCAM-1, MAdCAM-1, E-cadherin, JAM-1, JAM-2, JAM-3. In alternative embodiments, a targeting moiety can be an integrin or fragment or variant or homologue thereof expressed by leukocyte that binds to an integrin ligand. Examples of such integrins useful as targeting moieties for a endothelial delivery agent as disclosed herein can be, for example but not limited to; LFA-1 (αLβ2), Mac-1 (αMβ2), p150.95 (αXβ2), αDβ2, VLA-4 (α4β1), and β₇ (α4β7 and αEβ7) or fragments, homologues or variants thereof. An endothelial delivery agent as disclosed herein is useful to delivering agents to endothelial cells contributing to a pathogenesis, such as abnormal or aberrant angiogenesis and/or inflamed endothelial cells.

As disclosed herein, the inventors have developed a method to efficiently deliver drugs to leukocytes and endothelial cells using a platform technology, integrin/integrin ligand-targeted stabilized nano-particles (I/IL-tsNP). In some embodiments, I/IL-tsNP comprises a ˜100 nm-diameter unilameller liposome on which two functional layers are constructed. As disclosed herein, in the first layer, hyaluronan is covalently attached to lipid. Hyaluronan stabilized liposome and serves as a cryo-protectant that maintains the integrity of the structure of the particle during a cycle of lyophilization/re-hydration. Hyaluronan also enables long-circulation when particles are systemically delivered. In the second layer, specific antibodies are covalently attached to hyaluronan, functioning to direct particles to specific targets.

I/IL-tsNP is a systemically applicable drug delivery technology that can deliver not only hydrophilic- and lipophilic drugs individually, but also hydrophilic- and lipophilic drugs simultaneously. Hydrophilic drugs (e.g. nucleic acids) are encapsulated in the cavity inside of the particles, whereas lipophilic drugs (e.g. Taxol) are incorporated in the lipid bilayer of the particles. Simultaneous delivery of two drugs is expected to excerpt synergistic effects. As demonstrated by the simultaneous delivery of Taxol that perturbs micro-tubeles and interferes with mitosis and cyclin D1-siRNA that block the transition from G1 to S phase, two drugs that act on distinct sites in a same pathway will synergize to block the pathway.

### DEFINITIONS

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

The term “leukocyte-delivery agent” as used herein refers to the combination of targeting moiety which binds to integrins on the surface of leukocytes which is associated with a carrier particle, where the carrier particle can associate with at least one hydrophilic and/or at least one hydrophobic agent.

The term “endothelial cell delivery agent” as used herein refers to the combination of targeting moiety which binds to integrin ligands on the surface of endothelial cells which is associated with a carrier particle, where the carrier particle can associate with at least one hydrophilic and/or at least one hydrophobic agent.

The term “integrin” and “integrin receptor” are used interchangeably herein, broadly refers an integral membrane protein in the plasma membrane of cells. It plays a role in the attachment of a cell to the extracellular matrix (ECM) and to other cells, for example the attachment of leukocytes to endothelial cells. Integrins are obligate heterodimers containing two distinct chains, called the α (alpha) and β (beta) subunits. In mammals, 19 α and 8 β subunits have been characterized.

As used herein, the term “integrin ligand” refers to a member of the immunoglobulin superfamily ligands or “IgSF” Ig (immunoglobulin) superfamily (IgSF) of cellular adhesion molecules (CAMs). Immunoglobulin superfamily CAMs (IgSF CAMs) are either homophilic or heterophilic and bind integrins or different IgSF CAMs. Examples of integrin ligands are; NCAMs (Neural Cell Adhesion Molecules); Intracellular adhesion molecules (ICAMs); VCAM-1 (Vascular Cell Adhesion Molecule); PECAM-1 (Platelet-endothelial Cell Adhesion Molecule); L1; CHL1 and MAG. The interaction of a integrin ligand on the surface of an endothelial cell and an integrin on the surface of a leukocyte enables leukocyte transendothelial migration and leukocyte infiltration into a site of tissue damage or inflammation.

The term “active confirmation” refers to the conformation of a molecule, such as a protein or nucleic acid or drug that is capable of a functional biological effect. Stated another way and by way of an example, an integrin in the active confirmation is an integrin in a confirmation that is biologically active, as apposed to a biologically inactive confirmation.

The term “targeting moiety” or “targeting moiety” refers to an agent that homes in on or preferentially associates or binds to at least one of the following selected from; a particular tissue, cell type, cell surface marker, cell surface receptor, infecting agent or other area of interest, and the like. Examples of a targeting moiety includes, but are not limited to, an antibody, a oligonucleotide, an antigen, an antibody or functional fragment thereof, a ligand, a receptor, one member of a specific binding pair, a polyamide including a peptide having affinity for a biological receptor, an oligosaccharide, a polysaccharide, a steroid or steroid derivative, a hormone, e.g.; estradiol or histamine, a hormone-mimic, e.g., morphine, or other compound having binding specificity for a target. In the methods of the present invention, a targeting moiety promotes transport or preferential localization of a carrier particle to a target cell, for example a leukocyte or endothelial target cell.

A “marker” as used herein describes a characteristic and/or phenotype of a cell. Markers can be referred to as “cell-surface markers” and are often a cell-surface protein or glycoprotein expressed on the surface of a cell which can be used for binding of a targeting moiety to a target cell of interest. Markers will vary with specific cells. Markers are characteristics, whether morphological, functional or biochemical (enzymatic) characteristics particular to a cell type, or molecules expressed by the cell type. Preferably, such markers are proteins, and more preferably, possess an epitope for antibodies or other binding molecules available in the art. However, a cell marker can also be any molecule found within a cell including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate along particular lineages. Markers may be detected by any method available to one of skill in the art.

As used herein, an “antibody” or “functional fragment” of an antibody encompasses polyclonal and monoclonal antibody preparations, as well as preparations including hybrid or chimeric antibodies, such as humanized antibodies, altered antibodies, F(ab′)₂ fragments, F(ab) fragments, Fv fragments, single domain antibodies, dimeric and trimeric antibody fragment constructs, minibodies, and functional fragments thereof which exhibit immunological binding properties of the parent antibody molecule and/or which bind a cell surface antigen. The term “antibody” also encompasses antibodies and fragments thereof, for example monoclonal antibodies or monoclonal antibody fragments such as, for example, Fab and F(ab′)₂ receptor.

As used herein, the term “agent” refers to an agent that can be transported by the carrier particle and targeting moiety (i.e. an antibody to an integrin or an integrin ligand) to the target cell, for example a leukocyte target cell. An agent can be a chemical molecule of synthetic or biological origin. In some embodiments, an agent is generally a molecule that can be used in a pharmaceutical composition, for example the agent is a therapeutic agent. An agent as used herein also refers to any chemical entity or biological product, or combination of chemical entities or biological products, administered to a subject to treat or prevent or control a disease or condition, and are herein referred to as “therapeutic agents”. An agent for use in the invention as disclosed herein can affect the body therapeutically, or which can be used in vivo for diagnosis. Examples of therapeutic agents include chemotherapeutics for cancer treatment, antibiotics for treating infections, antifungals for treating fungal infections, therapeutic nucleic acids including nucleic acid analogs, e.g., siRNA. An agent can be a chemical entity or biological product, or combination of chemical entities or biological products, administered to a subject for imaging purposes in the subject, for example to monitor the presence or progression of disease or condition, and are herein referred to as “imaging agents” or “diagnostic agents”.

The term “agent” also typically refers to any entity which is normally not present or not present at the levels being administered in the target cell. Agent can be selected from a group comprising: chemicals; small molecules; nucleic acid sequences; nucleic acid analogues; proteins; peptides; aptamers; antibodies; or fragments thereof. A nucleic acid sequence can be RNA or DNA, and can be single or double stranded, and can be selected from a group comprising; nucleic acid encoding a protein of interest, oligonucleotides, nucleic acid analogues, for example peptide-nucleic acid (PNA), pseudo-complementary PNA (pc-PNA), locked nucleic acid (LNA) etc. Such nucleic acid sequences include, for example, but are not limited to, nucleic acid sequence encoding proteins, for example that act as transcriptional repressors, antisense molecules, ribozymes, small inhibitory nucleic acid sequences, for example but are not limited to RNAi, shRNAi, siRNA, micro RNAi (mRNAi), antisense oligonucleotides etc. A protein and/or peptide or fragment thereof can be any protein of interest, for example, but are not limited to: mutated proteins; therapeutic proteins and truncated proteins, wherein the protein is normally absent or expressed at lower levels in the cell. Proteins can also be selected from a group comprising; mutated proteins, genetically engineered proteins, peptides, synthetic peptides, recombinant proteins, chimeric proteins, antibodies, midibodies, minibodies, triabodies, humanized proteins, humanized antibodies, chimeric antibodies, modified proteins and fragments thereof. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be selected from a library of diverse compounds.

The term “hydrophilic” as used herein refers to a molecule or portion of a molecule that is typically charge-polarized and capable of hydrogen bonding, enabling it to dissolve more readily in water than in oil or other hydrophobic solvents. Hydrophilic molecules are also known as polar molecules and are molecules that readily absorb moisture, are hygroscopic, and have strong polar groups that readily interact with water. A “hydrophilic” polymer as the term is used herein, has a solubility in water of at least 100 mg/ml at 25° C.

The term “soluble agent” or “hydrophilic agent” and “hydrophilic drug” are used interchangeably herein, refers to any organic or inorganic compound or substance having biological or pharmacological activity and adapted or used for a therapeutic purpose having a water solubility greater than 10 mg/ml.

The term “hydrophobic” as used herein refers molecules tend to be non-polar and prefer other neutral molecules and non-polar solvents. Hydrophobic molecules in water often cluster together. Water on hydrophobic surfaces will exhibit a high contact angle. Examples of hydrophobic molecules include the alkanes, oils, fats, and greasy substances in general. Hydrophobic materials are used for oil removal from water, the management of oil spills, and chemical separation processes to remove non-polar from polar compounds. Hydrophobic molecules are also known as non-polar molecules. Hydrophobic molecules do not readily absorb water or are adversely affected by water, e.g., as a hydrophobic colloid. A “hydrophobic” polymer as the term is used herein has a solubility in water less than 10 mg/ml at 25° C., preferably less than 5 mg/ml, less than 1 mg/ml or lower.

The term “lipophilic” as used herein is used to refer to a molecule having an affinity for lipid molecules or fat molecules, pertaining to or characterized by lipophilia. Lipophilic or fat-liking molecules refers to molecules with an ability to dissolve in fats, oils, lipids, and non-polar solvents, for example such as hexane or toluene. Lipophilic substances tend to dissolve in other lipophilic substances, while hydrophilic (water-loving) substances tend to dissolve in water and other hydrophilic substances. Lipophilicity, hydrophobic and non-polarity (the latter as used to describe intermolecular interactions and not the separation of charge in dipoles) all essentially describe the same molecular attribute; the terms are often used interchangeably

The term “insoluble agent” or “hydrophobic agent” or “hydrophobic drug” are used interchangeably herein, refers to any organic or inorganic compound or substance having biological or pharmacological activity and adapted or used for a therapeutic purpose having a water solubility of less than 10 mg/ml. Typically an insoluble agent is an agent which is water insoluble, poorly water soluble, or poorly soluble in such as those agents having poor solubility in water at or below normal physiological temperatures, that is having at least less than 10 mg/ml, such as about <5 mg/ml at physiological pH (6.5-7.4), or about <1 mg/ml, or about <0.1 mg/ml.

The term “aqueous solution” as used herein includes water without additives, or aqueous solutions containing additives or excipients such as pH buffers, components for tonicity adjustment, antioxidants, preservatives, drug stabilizers, etc., as commonly used in the preparation of pharmaceutical formulations.

The term “leukocyte” as used herein refers to a white blood cell, including but not limited to polymorphonuclear neutrophil (polymorphs), lymphocyte, eosinophil, and basophile. Each leukocyte is characterized by different proteins or glucoproteins or receptors present on their surface and perform different functions.

The term “endothelial cell” as used herein refers to cells that line the inside surfaces of body cavities, blood vessels, and lymph vessels and making up the endothelium. Endothelial cells are typically but not necessarily thin, flattened cells.

The term “synergy” or “synergistic” as used herein, refers to the interaction of two or more agents so that their combined effect is greater than each of their individual effects at the same dose alone.

The term “additive” as used herein in the context of one agent has an additive effect on a second agent, refers to an increase in effectiveness of a first agent in the presence of a second agent as compared to the use of the first agent alone. Stated in another way, the second agent can function as an agent which enhances the physiological response of an organ or organism to the presence of a first agent. Thus, a second agent will increase the effectiveness of the first agent by increasing an individuals response to the presence of the first agent.

As used herein, the term “target cells” is used herein to refer to a cell which has cell surface molecules or markers which bind to, or have affinity for the targeting moiety. A target cell as used herein is any cell in which it is desirable to deliver a insoluble agent and a soluble agent to, and which can be selectively targeted by, or bind a targeting moiety as disclosed herein. In some aspects of the present invention, a target cell is a leukocyte. In an alternative embodiment, a target cell can be an endothelial cell. In some embodiments, target cells of the present invention have integrins molecules, variants, fragments or homologues thereof present on their cell surface. In one embodiment, the target cells are leukocytes which express at least one of the following integrins LFA-1 (αLβ2), Mac-1 (αMβ2), p150.95 (αXβ2), αDβ2, VLA-4 (α4β1), and β₇ (α4β7 and αEβ7).

The term “selectively target” as used herein refers to the ability of a targeting moiety to home in on or bind to a target cell with a greater affinity than to non-target cells. For example, about 10%, about 20%, about 30%, about 40%, preferably about 50%, more preferably about 60%, more preferably about 70%, still more preferably about 80%, still more preferably about 90%, still more preferably about 100% or greater affinity for the target cell relative to non-target cells.

The term “targeting moiety” or “target moiety” are used interchangeably herein refers to a molecule which has affinity, or binds to a molecule on the surface of a target cell. A targeting moiety can be any molecule, for example but not limited to, antibodies, proteins, peptides, protein-binding partners, co-factors, small molecules, glycoproteins, lipids and fragments, analogues and variants thereof. As disclosed herein, a target moiety can bind to an integrin or an integrin ligand on the surface of the target cell, i.e. the surface of the leukocyte or endothelial cell respectively. Accordingly, a target moiety component of a leukocyte delivery agent as disclosed herein is useful for delivering an agent and selective targeting cells expressing integrins LFA-1 (αLβ2), Mac-1 (αMβ2), p150.95 (αXβ2), αDβ2, VLA-4 (α4β1), or β₇ (α4β7 and αEβ7). Accordingly, a target moiety component of an endothelial delivery agent as disclosed herein selectively targets cells expressing integrin ligands, such as, for example ICAM-1, ICAM-2, ICAM-3, VCAM-1, MAdCAM-1, E-cadherin, JAM-1, JAM-2 and JAM-3.

The term “I-tsNP” is used interchangeably herein with “integrin targeted and stabilized nanoparticle” is used to refer to leukocyte delivery agent comprising a leukocyte targeting moiety targeting integrins associated with a carrier particle, such as for example a nanoparticle that comprises a stabilizing agent or cryoprotectant such as but not limited to HA. Similarly, the term “IL-tsNP” is used interchangeably herein with “integrin ligand-targeted and stabilized nanoparticle” is used to refer to a endothelial delivery agent comprising a endothelial cell targeting moiety targeting integrin ligands, associated with a carrier particle, such as for example a nanoparticle that comprises a stabilizing agent or cryoprotectant such as but not limited to HA.

The term “carrier particle” as used herein refers to any entity with the capacity to associate with and carry (or transport) an agent in the body. As discussed herein, a carrier particle can carry both an insoluble agent and an soluble agent simultaneously. In alternative embodiments, a carrier particle can carry an insoluble agent or a soluble agent. Carrier particles can be a lipid particle, such as but not limited to a liposome or a protein or peptide carrier particle. Carrier particles as disclosed herein include any carrier particle modifiable by attachment of a targeting moiety known to the skilled artisan. Carrier particles include but are not limited to liposomal or polymeric nanoparticles such as liposomes, proteins, and non-protein polymers. Carrier particles can be selected according to (i) their ability to transport the agent of choice and (ii) the ability to associate with a targeting moiety as disclosed herein.

The term “nanoparticle” as used herein refers to a microscopic particle whose size is measured in nanometers. A carrier particle here can be a nanoparticle.

The term “lipid particle” refers to lipid vesicles such as liposomes or micelles.

The term “micelle” as used herein refers to an arrangement of surfactant molecules (surfactants comprise a non-polar, lipophilic “tail” and a polar, hydrophilic “head”). As the term is used herein, a micelle has the arrangement in aqueous solution in which the non-polar tails face inward and the polar heads face outward. Micelles are typically colloid particles formed by an aggregation of small molecules and are usually microscopic particles suspended in some sort of liquid medium, e.g., water, and are between one nanometer and one micrometer in size. A typical micelle in aqueous solution forms an aggregate with the hydrophilic “head” regions in contact with surrounding solvent, sequestering the hydrophobic tail regions in the micelle center. This type of micelle is known as a normal phase micelle (oil-in-water micelle). Inverse micelles have the headgroups at the centre with the tails extending out (water-in-oil micelle). Micelles are approximately spherical in shape. Other phases, including shapes such as ellipsoids, cylinders, and bilayers are also possible. The shape and size of a micelles a function of the molecular geometry of its surfactant molecules and solution conditions such as surfactant concentration, temperature, pH, and ionic strength. The process of forming micellae is known as micellisation.

The term “polymer” as used herein, refers to a linear chain of two or more identical or non-identical subunits joined by covalent bonds. A peptide is an example of a polymer that can be composed of identical or non-identical amino acid subunits that are joined by peptide linkages.

The term “stabilized liposome” as used herein refers to a liposome that comprises a cryoprotectant and/or a long-circulating agent.

The terms “encapsulation” and “entrapped,” as used herein, refer to the incorporation of an agent in a lipid particle. Ari agent can be present in the aqueous interior of the lipid particle, for example a hydrophilic agent. In one embodiment, a portion of the encapsulated agent takes the form of a precipitated salt in the interior of the liposome. The agent may also self precipitate in the interior of the liposome. In alternative embodiments, an agent can be incorporated into the lipid phase of a carrier particle, for example a hydrophobic and/or lipophilic agent.

The term “protein” as used herein, refers to a compound that is composed of linearly arranged amino acids linked by peptide bonds, but in contrast to peptides, has a well-defined conformation. Proteins, as opposed to peptides, generally consist of chains of 50 or more amino acids.

The incorporation of non-natural amino acids, including synthetic non-native amino acids, substituted amino acids, or one or more D-amino acids into the peptides (or other components of the composition, with exception for protease recognition sequences) is desirable in certain situations. D-amino acid-containing peptides exhibit increased stability in vitro or in vivo compared to L-amino acid-containing forms. Thus, the construction of peptides incorporating D-amino acids can be particularly useful when greater in vivo or intracellular stability is desired or required. More specifically, D-peptides are resistant to endogenous peptidases and proteases, thereby providing better oral trans-epithelial and transdermal delivery of linked drugs and conjugates, improved bioavailability of membrane-permanent complexes (see below for further discussion), and prolonged intravascular and interstitial lifetimes when such properties are desirable. The use of D-isomer peptides can also enhance transdermal and oral trans-epithelial delivery of linked drugs and other cargo molecules. Additionally, D-peptides cannot be processed efficiently for major histocompatibility complex class II-restricted presentation to T helper cells, and are therefore less likely to induce humoral immune responses in the whole organism. Peptide conjugates can therefore be constructed using, for example, D-isomer forms of cell penetrating peptide sequences, L-isomer forms of cleavage sites, and D-isomer forms of therapeutic peptides.

The term “derivative” as used herein refers to polypeptides, peptides and antibodies which have been chemically modified, for example but not limited to by techniques such as ubiquitination, labeling, pegylation (derivatization with polyethylene glycol) or addition of other molecules.

As used herein, “variant” with reference to a polynucleotide or polypeptide, refers to a polynucleotide or polypeptide that can vary in primary, secondary, or tertiary structure, as compared to a reference polynucleotide or polypeptide, respectively (e.g., as compared to a wild-type polynucleotide or polypeptide). A “variant” of an integrin, for example a LFA-1 is meant to refer to a molecule substantially similar in structure and function, i.e. where the function is the ability to bind to a LFA-1 integrin ligand, such as ICAM-1 on endothelial cells, to either the entire molecule, or to a fragment thereof. A molecule is said to be “substantially similar” to another molecule if both molecules have substantially similar structures or if both molecules possess a similar biological activity. Thus, provided that two molecules possess a similar activity, they are considered variants as that term is used herein even if the structure of one of the molecules not found in the other, or if the sequence of amino acid residues is not identical.

The term “functional derivative” or “functional fragment” or “mimetic” are used interchangeably herein, and refers to a molecule or compound which possess a biological activity (either functional or structural) that is substantially similar to a biological activity of the entity or molecule its is a functional derivative of. The term functional derivative is intended to include the fragments, variants, analogues or chemical derivatives of a molecule.

The term “fragment” of a polypeptide, protein or peptide or molecule as used herein refers to any contiguous polypeptide subset of the molecule. Fragments of an antibody or an integrin ligand, such as, for example a fragment of ICAM-1 has the same binding affinity for its integrin binding partner, such as LFA-1 on the surface of the leukocyte as that of the full length ICAM-1. Stated another way, a fragment of an integrin ligand, such as, for example a fragment of ICAM-1 is a fragment of ICAM-1 which can bind with the same, or lower or higher affinity to its ligand. Fragments as used herein typically are soluble (i.e. not membrane bound).

Fragments of an ICAM-1 peptide, for example functional fragments of LFA-1 useful in the methods as disclosed herein have at least 30% of agonist or antagonist activity as that of LFA-1. Stated another way, a fragment or functional fragment of an ICAM-1 peptide which result in at least 30% of the same activity as compared to full length peptide, for example functional fragments of ICAM-1 to bind to its integrin binding partner LFA-1. It can also include fragments that decrease the wild type activity of one property by at least 30%. Fragments as used herein are soluble (i.e. not membrane bound). A “fragment” can be at least about 6, at least about 9, at least about 15, at least about 20, at least about 30, least about 40, at least about 50, at least about 100, at least about 250, at least about 500 nucleic or amino acids, and all integers in between. Exemplary fragments include C-terminal truncations, N-terminal truncations, or truncations of both C- and N-terminals (e.g., deletions of, for example, at least 1, at least 2, at least 3, at least 4, at least 5, at least 8, at least 10, at least 15, at least 20, at least 25, at least 40, at least 50, at least 75, at least 100 or more amino acids deleted from the N-termini, the C-termini, or both). One of ordinary skill in the art can create such fragments by simple deletion analysis. Such a fragment of ICAM-1 or LFA-1 can be, for example, 1, 2, 3, 4, 5, 6, 7, 8, 9 or 10 amino acids or more than 10 amino acids, such as 15, 30, 50, 100 or more than 100 amino acids deleted from the N-terminal and/or C-terminal amino acids of an integrin or integrin ligand as those proteins are defined herein. Persons of ordinary skill in the art can easily identify the minimal peptide fragment of an integrin and/or integrin ligand useful as targeting agents and in the compositions and methods as disclosed herein, by sequentially deleting N- and/or C-terminal amino acids from the integrin and/or integrin ligand and assessing the function of the resulting peptide fragment to bind their respective binding partner, i.e. of a fragment integrin to bind its respective integrin ligand, and/or an integrin ligand fragment to bind its integrin. One can create functional fragments with multiple smaller fragments. These can be attached by bridging peptide linkers. One can readily select linkers to maintain wild type conformation. In some embodiments, a fragment of integrin and/or integrin ligands can be less than 200, or less than 150 or less than 100, or less than 50, or less than 20 amino acids of the full length integrin and/or integrin ligand. In some embodiments, a fragment of an integrin and/or integrin ligand is less than 100 peptides in length. However, as stated above, the fragment must be at least 6 amino acids, at least about 9, at least about 15, at least about 20, at least about 30, at least about 40, at least about 50, at least about 100, at least about 250, at least about 500 nucleic acids or amino acids, or any integers in between.

As used herein, “homologous” or “homologues” are used interchangeably, and when used to describe a polynucleotide or polypeptide, indicates that two polynucleotides or polypeptides, or designated sequences thereof, when optimally aligned and compared, are identical, with appropriate nucleotide insertions or deletions or amino-acid insertions or deletions, in at least 70% of the nucleotides, usually from about 75% to 99%, and more preferably at least about 98 to 99% of the nucleotides. The term “homolog” or “homologous” as used herein also refers to homology with respect to structure and/or function. With respect to sequence homology, sequences are homologs if they are at least about 50%, at least about 70%, at least about 80%, at least about 90%, at least about 95% identical, at least about 97% identical, or at least about 99% identical. The term “substantially homologous” refers to sequences that are at least about 90%, at least about 95% identical, at least about 97% identical or at least about 99% identical. Homologous sequences can be the same functional gene in different species.

Determination of homologs of the genes or peptides of the present invention can be easily ascertained by the skilled artisan. The terms “homology” or “identity” or “similarity” are used interchangeably herein and refers to sequence similarity between two peptides or between two nucleic acid molecules. Homology and identity can each be determined by comparing a position in each sequence which can be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences. A sequence which is “unrelated” or “non-homologous” shares less than 40% identity, though preferably less than 25% identity with a sequence of the present application.

In one embodiment, the term “integrin ligand homolog” refers to an amino acid sequence that has 40% homology to at least a region of the full length amino acid sequence of the integrin ligand to which it is homologous to, for example a ICAM-1 receptor homologue is at least 40% homologous to a region of the full length amino acid sequence of ICAM-1, more preferably at least about 50%, still more preferably, at least about 60% homology, still more preferably, at least about 70% homology, even more preferably, at least about 75% homology, yet more preferably, at least about 80% homology, even more preferably at least about 85% homology, still more preferably, at least about 90% homology, and more preferably, at least about 95% homology. As discussed above, the homology is at least about 50% to 100% and all intervals in between (i.e., 55%, 60%, 70%, 75%, 80%, 85%, 90%, 95%, 98%, etc.).

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, for example, by the local homology algorithm of Smith and Waterman (Adv. Appl. Math. 2:482 (1981), which is incorporated by reference herein), by the homology alignment algorithm of Needleman and Wunsch (J. Mol. Biol. 48:443-53 (1970), which is incorporated by reference herein), by the search for similarity method of Pearson and Lipman (Proc. Natl. Acad. Sci. USA 85:2444-48 (1988), which is incorporated by reference herein), by computerized implementations of these algorithms (e.g., GAP, BESTFIT, FASTA, and TFASTA in the Wisconsin Genetics Software Package, Genetics Computer Group, 575 Science Dr., Madison, Wis.), or by visual inspection. (See generally Ausubel et al. (eds.), Current Protocols in Molecular Biology, 4th ed., John Wiley and Sons, New York (1999)).

One example of a useful algorithm is PILEUP. PILEUP creates a multiple sequence alignment from a group of related sequences using progressive, pairwise alignments to show the percent sequence identity. It also plots a tree or dendogram showing the clustering relationships used to create the alignment. PILEUP uses a simplification of the progressive alignment method of Feng and Doolittle (J. Mol. Evol. 25:351-60 (1987), which is incorporated by reference herein). The method used is similar to the method described by Higgins and Sharp (Comput. Appl. Biosci. 5:151-53 (1989), which is incorporated by reference herein). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of an algorithm that is suitable for determining percent sequence identity and sequence similarity is the BLAST algorithm, which is described by Altschul et al. (J. Mol. Biol. 215:403-410 (1990), which is incorporated by reference herein). (See also Zhang et al., Nucleic Acid Res. 26:3986-90 (1998); Altschul et al., Nucleic Acid Res. 25:3389-402 (1997), which are incorporated by reference herein). Software for performing BLAST analyses is publicly available through the National Center for Biotechnology Information internet web site. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990), supra). These initial neighborhood word hits act as seeds for initiating searches to find longer HSPs containing them. The word hits are then extended in both directions along each sequence for as far as the cumulative alignment score can be increased. Extension of the word hits in each direction is halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a word length (W) of 11, the BLOSUM62 scoring matrix (see Henikoff and Henikoff, Proc. Natl. Acad. Sci. USA 89:10915-9 (1992), which is incorporated by reference herein) alignments (B) of 50, expectation (E) of 10, M=5, N=−4, and a comparison of both strands.

In addition to calculating percent sequence identity, the BLAST algorithm also performs a statistical analysis of the similarity between two sequences (see, e.g., Karlin and Altschul, Proc. Natl. Acad. Sci. USA 90:5873-77 (1993), which is incorporated by reference herein). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more typically less than about 0.01, and most typically less than about 0.001.

As used herein, “gene silencing” or “gene silenced” in reference to an activity of n RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, the term “RNAi” refers to any type of interfering RNA, including but are not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). RNAi molecules as used herein are any interfering RNA, or RNA interference molecules, such as nucleic acid molecules or analogues thereof for example RNA-based molecules that inhibit gene expression. RNAi refers to a means of selective post-transcriptional gene silencing. RNAi, for example use of an siRNA can result in the destruction of specific mRNA, or prevents the processing or translation of RNA, such as mRNA.

The term “short interfering RNA” (siRNA), also referred to herein as “small interfering RNA” is defined as an agent which functions to inhibit expression of a target gene, e.g., by RNAi. An siRNA can be chemically synthesized, it can be produced by in vitro transcription, or it can be produced within a host cell. siRNA molecules can also be generated by cleavage of double stranded RNA, where one strand is identical to the message to be inactivated.

The term “therapeutically effective amount” refers to an amount that is sufficient to effect a therapeutically or prophylactically significant reduction in a symptom associated with an angiogenesis-mediated condition when administered to a typical subject who has an angiogenesis-mediated condition. A therapeutically or prophylatically significant reduction in a symptom is, e.g. about 10%, about 20%, about 30%, about 40%, about 50%, about 60%, about 70%, about 80%, about 90%, about 100%, about 125%, about 150% or more as compared to a control or non-treated subject. In some embodiments where the angiogenesis-mediated condition is cancer, the term “therapeutically effective amount” refers to the amount that is safe and sufficient to prevent or delay the development and further spread of metastases in cancer patients. The amount can also cure or cause the cancer to go into remission, slow the course of cancer progression, slow or inhibit tumor growth, slow or inhibit tumor metastasis, slow or inhibit the establishment of secondary tumors at metastatic sites, or inhibit the formation of new tumor metastasis.

As used herein, the terms “treating” or “treatment” of a disease include preventing the disease, i.e. preventing a clinical symptom of the disease in a subject that can be exposed to, or predisposed to, a disease, but does not yet experience or display a symptom of the disease; inhibiting a disease, i.e., arresting the development of a disease or a clinical symptom of the disease; or relieving a disease, i.e., causing regression of a disease or a clinical symptom of the disease. The term “treat” or “treatment” refer to both therapeutic treatment and prophylactic or preventative measures, wherein the object is to prevent or slow down the development or spread of a disease. Beneficial or desired clinical results include, but are not limited to, alleviation of a symptoms, diminishment of extent of a disease, stabilized (i.e., not worsening) state of a disease, delay or slowing of the disease progression, amelioration or palliation of a disease state, and remission (whether partial or total), whether detectable or undetectable. “Treatment” can also mean prolonging survival as compared to expected survival if not receiving treatment.

The term “effective amount” as used herein refers to the amount of therapeutic agent of pharmaceutical composition to alleviate at least some of the symptoms of the disease or disorder. The term “effective amount” includes within its meaning a sufficient amount of pharmacological composition to provide the desired effect. The exact amount required will vary depending on factors such as the type of tumor to be treated, the severity of the tumor, the drug resistance level of the tumor, the species being treated, the age and general condition of the subject, the mode of administration and so forth. Thus, it is not possible to specify the exact “effective amount”. However, for any given case, an appropriate “effective amount” can be determined by one of ordinary skill in the art using only routine experimentation.

The terms “composition” or “pharmaceutical composition” are used interchangeably herein and refers to compositions or formulations that usually comprise an excipient, such as a pharmaceutically acceptable carrier that is conventional in the art and that is suitable for administration to mammals, and preferably humans or human cells. Such compositions can be specifically formulated for administration via one or more of a number of routes, including but not limited to, oral, parenteral, intravenous, intraarterial, subcutaneous, intranasal, sublingual, intraspinal, intracerebroventricular, and the like. Cells administered a composition as disclosed herein can be part of a subject, for example for therapeutic, diagnostic, or prophylactic purposes. The cells can also be cultured, for example cells as part of an assay for screening potential pharmaceutical compositions, and the cells can be part of a transgenic animal for research purposes. In addition, compositions for topical (e.g., oral mucosa, respiratory mucosa) and/or oral administration can form solutions, suspensions, tablets, pills, capsules, sustained-release formulations, oral rinses, or powders, as known in the art are described herein. The compositions also can include stabilizers and preservatives: For examples of carriers, stabilizers and adjuvants, University of the Sciences in Philadelphia (2005) Remington: The Science and Practice of Pharmacy with Facts and Comparisons, 21st Ed. The terms “composition” or “pharmaceutical composition” are used interchangeably herein and refers to compositions or formulations that usually comprise an excipient, such as a pharmaceutically acceptable carrier that is conventional in the art and that is suitable for administration to mammals, and preferably humans or human cells. Such compositions can be specifically formulated for administration via one or more of a number of routes, including but not limited to, oral, ocular and nasal administration and the like.

The “pharmaceutically acceptable carrier” means any pharmaceutically acceptable means to mix and/or deliver the targeted delivery composition to a subject. The term “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation and is compatible with administration to a subject, for example a human. For the clinical use of the methods of the present invention, targeted delivery composition of the invention is formulated into pharmaceutical compositions or pharmaceutical formulations for parenteral administration, e.g., intravenous; mucosal, e.g., intranasal; enteral, e.g., oral; topical, e.g., transdermal; ocular, e.g., via corneal scarification or other mode of administration. The pharmaceutical composition contains a compound of the invention in combination with one or more pharmaceutically acceptable ingredients. The carrier can be in the form of a solid, semi-solid or liquid diluent, cream or a capsule. These pharmaceutical preparations are a further object of the invention. Usually the amount of active compounds is between 0.1-95% by weight of the preparation, preferably between 0.2-20% by weight in preparations for parenteral use and preferably between 1 and 50% by weight in preparations for oral administration. The “pharmaceutically acceptable carrier” means any pharmaceutically acceptable means to mix and/or deliver the targeted delivery composition to a subject. The term “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation and is compatible with administration to a subject, for example a human. A diblock copolymer as described herein is a pharmaceutically acceptable carrier as the term is used herein. Other pharmaceutically acceptable carriers can be used in combination with the block copolymer carriers as described herein.

The term “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and infrasternal injection and infusion. The phrases “systemic administration,” “administered systemically,” “peripheral administration” and “administered peripherally” as used herein mean the administration of a compound, drug or other material other than directly into the central nervous system, such that it enters the animal's system and, thus, is subject to metabolism and other like processes, for example, subcutaneous administration.

As used herein, the terms “administering,” and “introducing” are used interchangeably herein and refer to the placement of the pharmaceutical composition comprising a leukocyte delivery agent and/or a endothelial delivery agent the and associated agents of the present invention into a subject by a method or route which results in at least partial localization of the agents at a desired site. The agents of the present invention can be administered by any appropriate route which results in an effective treatment in the subject.

The term “disease” or “disorder” is used interchangeably herein, and refers to any alteration in state of the body or of some of the organs, interrupting or disturbing the performance of the functions and/or causing symptoms such as discomfort, dysfunction, distress, or even death to the person afflicted or those in contact with a person. A disease or disorder can also relate to a distemper, ailing, ailment, malady, disorder, sickness, illness, complaint, inderdisposion or affectation.

The term “dyscrasias” as used herein is a nonspecific term that refers to any disease or disorder, although it usually refers to blood diseases, for example the term “plasma cell dyscrasias” as used herein refers to disorders of the plasma cells.

The term ‘malignancy’ and ‘cancer’ are used interchangeably herein, refers to diseases that are characterized by uncontrolled, abnormal growth of cells and also refers to any disease of an organ or tissue in mammals characterized by poorly controlled or uncontrolled multiplication of normal or abnormal cells in that tissue and its effect on the body as a whole. Cancer cells can spread locally or through the bloodstream and lymphatic system to other parts of the body. Cancer diseases within the scope of the definition comprise benign neoplasms, dysplasias, hyperplasias as well as neoplasms showing metastatic growth or any other transformations like e.g. leukoplakias which often precede a breakout of cancer. A ‘malignant cell’ as used herein is intended to refer to the cancer causing cell, or a cell that has uncontrolled proliferation. The term “cancer”, as used herein refers to a cellular proliferative disease in a human or animal subject.

The term “tumor” or “tumor cell” used interchangeably herein refers to the tissue mass or tissue type or cell type that is undergoing uncontrolled proliferation.

The term “degenerative disease” refers to a disease that has a progressive loss of function of an organ or tissue. Degenerative diseases that are the result of the degeneration of an organ or tissue typically begin at the onset of a symptom of the disease followed by a progressive increase in at least one symptom of the disease. Examples of degenerative diseases include but are not limited to, multiple sclerosis, neurological disorders such as Alzheimer's disease, ALS, progressive inflammation, and progressive arthritis. For example, the degenerative inflammation refers to a local reaction to injury, occasionally observed in the walls of blood vessels and in parenchymal cells of various organs in reacting to certain chemicals, viruses, and other intracellular agents; the response is characterized by degenerative changes in the cytoplasm and nucleus, frequently resulting in necrosis, but exudation (if any) is ordinarily observed only in the wall of the affected vessel, or in the interstices immediately adjacent to the affected vessel or parenchymal cells. Progressive arthritis is a form of arthritis that results in the destruction of the articular cartilage that line the joints. Seen predominately in the larger weight bearing joints of the hips, knees and spine, but may also be evident in the small joints of the hands.

The term “autoimmune disease” is used interchangeably herein with “immune response mediated disorder” and refers to disorders in which the hosts' immune system contributes to the disease condition either directly or indirectly. Examples of disorders which are mediated by the immune response includes AIDS, autoimmune disease, and graft rejection or graft versus host (GVH) disease. As used herein, graft rejection encompasses both host versus graft and graft versus host rejection.

As used herein, the term “medicament” refers to an agent that promotes the recovery from and/or alleviate a symptoms of an angiogenesis-mediated condition.

As used herein, the term “patient” refers to a human in need of the treatment to be administered.

The term “subject” and “individual” are used interchangeably herein, and refer to an animal, for example a human, to whom treatment, including prophylactic treatment, with a composition as described herein, is provided. The term “mammal” is intended to encompass a singular “mammal” and plural “mammals,” and includes, but is not limited: to humans, primates such as apes, monkeys, orangutans, and chimpanzees; canids such as dogs and wolves; felids such as cats, lions, and tigers; equids such as horses, donkeys, and zebras, food animals such as cows, pigs, and sheep; ungulates such as deer and giraffes; rodents such as mice, rats, hamsters and guinea pigs; and bears. Preferably, the mammal is a human subject. As used herein, a “subject” refers to a mammal, preferably a human. The term “individual”, “subject”, and “patient” are used interchangeably. Preferably, the mammal is a human subject.

In this specification and the appended claims, the singular forms “a,” “an,” and “the” include plural references unless the context clearly dictates otherwise. Thus, for example, reference to a composition for delivering “a drug” includes reference to two or more drugs. In describing and claiming the present invention, the following terminology will be used in accordance with the definitions set out below.

The articles “a” and “an” are used herein to refer to one or to more than one (i.e., to at least one) of the grammatical object of the article. By way of example, “an element” means one element or more than one element.

Other than in the operating examples, or where otherwise indicated, all numbers expressing quantities of ingredients or reaction conditions used herein should be understood as modified in all instances by the term “about.” The term “about” when used in connection with percentages can mean±1%. The present invention is further explained in detail by the following examples, but the scope of the invention should not be limited thereto.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

### General

The present invention relates to methods and compositions for the simultaneous delivery of at least one insoluble agent and at least one soluble agent to a cell. In some embodiments, the inventors have discovered methods and compositions for the delivery of an insoluble agent and a soluble agent to a target cell.

Accordingly, one aspect of the present invention relates to a composition for the simultaneous delivery of an insoluble agent and a soluble agent to a target cell, wherein the composition comprises a carrier particle comprising an insoluble agent and/or a soluble agent, wherein the carrier particle is attached or conjugated to a targeting moiety, where the targeting moiety binds to and has specific affinity for to a cell surface marker on the target cell (i.e. the targeting moiety selectively targets the target cell).

A target cell can be any cell from any species, for example mammalian species, and in some embodiments a target cell is a human target cell. One can target any cell or target any cell type where it is desirable to have the simultaneous delivery of an insoluble agent and a soluble agent. Without being limited to exemplary examples, the inventors have demonstrated delivery of an insoluble agent and a soluble agent to leukocytes and endothelial cells. As disclosed herein and in Example 7, the inventors demonstrate targeted delivery of an insoluble agent (taxol) and a soluble agent (RNAi) to leukocytes using either anti-integrin antibody-coated carrier particles or integrin ligand-coated carrier particles. The inventors have also demonstrated targeted delivery of an insoluble agent and/or a soluble agent to endothelial cells using anti-integrin ligand antibody-coated carrier particles or integrin-coated carrier particles.

Accordingly, in some embodiments, the target cell is a leukocyte. One aspect of the present invention related to a method to deliver at least one hydrophilic (or soluble) agent and at least one hydrophobic (or insoluble) agent to a leukocyte, by contacting the leukocyte with a carrier particle comprising an insoluble agent (or hydrophobic agent) and a soluble agent (hydrophilic agent), wherein the carrier particle is conjugated to a targeting moiety which binds to or has high affinity for cell surface integrins on the leukocytes, thereby selectively targeting the carrier particle to the leukocyte.

Accordingly, the present invention relates to compositions and methods for the simultaneous delivery of soluble and insoluble agents to leukocytes, where the composition is “leukocyte delivery agent” and comprises associating a lymphocyte targeting moiety to a carrier particle, where the targeting moiety has affinity for, or binds to integrins present on the surface of leukocytes, and where the soluble and insoluble agents are associated with the carrier particle.

In some embodiments, a targeting moiety useful in leukocyte delivery agent as disclosed herein has affinity for, or binds to integrins expressed in leukocytes, for example but not limited to of LFA-1 (αLβ2), Mac-1 (αMβ2), p150.95 (αXβ2), αDβ2, VLA-4 (α4β1), and β₇ (α4β7 and αEβ7). In some embodiments, such a targeting moiety is an antibody or fragment thereof with affinity for, or binds LFA-1 (αLβ2), Mac-1 (αMβ2), p150.95 (αXβ2), αDβ2, VLA-4 (α4β1), and β₇ (α4β7 and αEβ7). In alternative embodiments, a targeting moiety can be an integrin ligand or fragment or variant or homologue thereof that binds to such integrins, for example integrin ligands such as members of the IgSF (Ig Superfamily) of cell Adhesion molecules (CAM) expressed on endothelial cells, for example but not limited to, ICAM-1, ICAM-2, ICAM-3, VCAM-1, MAdCAM-1, E-cadherin, JAM-1, JAM-2, JAM-3 or fragments, homologues or variants thereof.

In some embodiments, an endothelial delivery agent is used to deliver agents to endothelial cells, which comprises a targeting moiety that has affinity for integrin ligands present on the surface of endothelial cells, where the targeting moiety is associated with a carrier particle.

In some embodiments, a targeting moiety useful in a endothelial delivery agent has affinity for integrin ligands on endothelial cells, for example a targeting moiety has affinity for, or binds to integrin ligands of the IgSF (Ig Superfamily) of cell Adhesion molecules (CAM), for example, antibodies or fragment thereof which bind to ICAM-1, ICAM-2, ICAM-3, VCAM-1, MAdCAM-1, E-cadherin, JAM-1, JAM-2, JAM-3. In alternative embodiments, a targeting moiety useful in a endothelial delivery agent can be an integrin binding partner or fragment or variant or homologue thereof expressed by a leukocyte that binds to an integrin ligand. Examples of such integrins useful as targeting moieties for a endothelial delivery agent as disclosed herein can be, for example but not limited to; LFA-1 (αLβ2), Mac-1 (αMβ2), p150.95 (αXβ2), αDβ2, VLA-4 (α4β1), and β₇ (α4β7 and αEβ7) or fragments, homologues or variants thereof. An endothelial delivery agent as disclosed herein is useful to delivering agents to endothelial cells contributing to a pathogenesis, such as abnormal or aberrant angiogenesis and/or inflamed endothelial cells.

In one aspect of the present invention, methods and compositions for delivery of agents to leukocytes are provided, comprising a simple, straightforward method of coating a carrier particle, for example a small lipid particles such as liposomes or micelles with a targeting moiety for leukocytes, wherein the carrier particle comprises at least one agent. In some embodiments, the carrier particle comprises a layer-by-layer lipid composition with a first layer of a cryoprotectant, such as hyaluronic acid and a second layer of a targeting moiety, e.g., antibody, scFv to an integrin on the leukocyte or a receptor leukocyte ligand. In some embodiments, methods for encapsulating hydrophobic agents or hydrophilic agents, or both, in the carrier particle associated with the targeting moiety is provided.

### Targeting Moieties

Targeting moieties useful in the methods and compositions of the present invention include, for example, antibodies, antibody fragments or antigen binding fragments and the like. In one embodiment an antibody can be a functional fragment containing the antigen binding region of the antibody. A preferred antibody fragment is a single chain Fv fragment of an antibody. The antibody or antibody fragment is one which will bind to an integrin (I) or integrin ligand (IL) on the surface of the target cell, and preferably integrins and/or integrin ligands that are differentially expressed on the target cell. In some embodiments, multiple different targeting moieties may be associated with the carrier particle.

In some embodiments, targeting moieties can selectively target leukocyte cells by specifically binding integrins that are exclusively or preferentially expressed on leukocytes. They can target activated leukocytes by targeting the leukocyte specific integrins in their active conformation. In some embodiments, leukocyte targeting moieties can be any molecule or entity which has specific affinity for or binds integrins. Examples of such molecules with specific affinity for integrins are, but are not limited to, integrin ligands or fragments, or variants or homologues thereof.

A targeting moiety useful in a leukocyte delivery agent as disclosed herein can be any molecule, entity, protein, peptide, antibody or antibody fragment that binds to, or has affinity for, an integrin present on the surface of a leukocyte. In particular, the targeting moiety is specific for binding to of having affinity for integrins LFA-1 (αLβ2), Mac-1 (αMβ2), p150.95 (αXβ2), αDβ2, VLA-4 (α4β1), and β₇ (α4β7 and αEβ7). In a particular embodiment, a targeting moiety is an antibody or fragment thereof which binds to LFA-1 (αLβ2) integrin.

Alternatively, a targeting moiety can be any molecule that binds to an integrin that is expressed on a target cell. For example a target moiety for a leukocyte delivery agent can be a integrin ligand or homologue or variant thereof, for example such a targeting moiety can be an integrin ligand for example but not limited to ICAM-1, ICAM-2, ICAM-3, VCAM-1, MAdCAM-1, E-cadherin, JAM-1, JAM-2, JAM-3 or fragments, homologues or variants thereof. In a particular embodiment, a targeting moiety is the integrin ligand ICAM-1 or a fragment, homologue or variant thereof. Sugar molecules or glycoproteins, lipid molecules or lipoproteins may be targeting moieties.

In alternative embodiments, a targeting moiety useful in an endothelial delivery agent as disclosed herein can be any molecule, protein, peptide, antibody or antibody fragment that binds to, or has affinity for, an integrin ligand present on the surface of an endothelial cell. In particular, the targeting moiety is specific for binding to of having affinity for integrin ligands ICAM-1, ICAM-2, ICAM-3, VCAM-1, MAdCAM-1, E-cadherin, JAM-1, JAM-2 and JAM-3. In a particular embodiment, a targeting moiety is an antibody or fragment thereof which binds to ICAM-1 integrin ligand.

Alternatively, a targeting moiety can be any molecule that binds to integrin that are specifically expressed on a target cell, for example a target moiety for an endothelial delivery agent can be an integrin or homologue or variant thereof present on a leukocyte, for example a targeting moiety can be selected from integrins; LFA-1 (αLβ2), Mac-1 (αMβ2), p150.95 (αXβ2), αDβ2, VLA-4 (α4β1), and β₇ (α4β7 and αEβ7) or homologues, fragments or variants thereof. Sugar molecules or glycoproteins, lipid molecules or lipoproteins may be targeting moieties.

Antibodies for use in the present invention can be produced using standard methods to produce antibodies, for example, by monoclonal antibody production (Campbell, A. M., Monoclonal Antibodies Technology: Laboratory Techniques in Biochemistry and Molecular Biology, Elsevier Science Publishers, Amsterdam, the Netherlands (1984); St. Groth et al., J. Immunology, (1990) 35: 1-21; and Kozbor et al., Immunology Today (1983) 4:72). Antibodies can also be readily obtained by using antigenic portions of the protein to screen an antibody library, such as a phage display library by methods well known in the art. For example, U.S. Pat. No. 5,702,892 (U.S.A. Health & Human Services) and WO 01/18058 (Novopharm Biotech Inc.) disclose bacteriophage display libraries and selection methods for producing antibody binding domain fragments.

By way of examples only, the production of non-human monoclonal antibodies, e.g., murine or rat, can be accomplished by, for example, immunizing the animal with an immunogenic peptide to which the antibody is to be desired, for example an antibody which binds to an integrin such as, for example but not limited to a protein or fragment thereof selected from the following group: LFA-1 (αLβ2), Mac-1 (αMβ2), p150.95 (αXβ2), ocDf32, VLA-4 (α4β1), and β₇ (α4β7 and αEβ7) or homologues thereof. See Harlow & Lane, Antibodies, A Laboratory Manual (CSHP NY, 1988) (incorporated by reference for all purposes). Such an immunogen can be obtained from a natural source, by peptides synthesis or by recombinant expression.

Humanized forms of mouse antibodies can be generated by linking the CDR regions of non-human antibodies to human constant regions by recombinant DNA techniques. See Queen et al., Proc. Natl. Acad. Sci. USA 86, 10029-10033 (1989) and WO 90/07861 (incorporated by reference for all purposes).

Human antibodies can be obtained using phage-display methods. See, e.g., Dower et al., WO 91/17271; McCafferty et al., WO 92/01047. In these methods, libraries of phage are produced in which members display different antibodies on their outersurfaces. Antibodies are usually displayed as Fv or Fab fragments. Phage displaying antibodies with a desired specificity are selected by affinity enrichment to immunoglobulin lambda 6 light chain or fragments thereof. Human antibodies against immunoglobulin lambda 6 light chain can also be produced from non-human transgenic mammals having transgenes encoding at least a segment of the human immunoglobulin locus and an inactivated endogenous immunoglobulin locus. See, e.g., Lonberg et al., WO93/12227 (1993); Kucherlapati, WO 91/10741 (1991) (each of which is incorporated by reference in its entirety for all purposes). Human antibodies can be selected by competitive binding experiments, or otherwise, to have the same epitope specificity as a particular mouse antibody. Such antibodies are particularly likely to share the useful functional properties of the mouse antibodies. Human polyclonal antibodies can also be provided in the form of serum from humans immunized with an immunogenic agent. Optionally, such polyclonal antibodies can be concentrated by affinity purification using a region of the immunoglobulin lambda light chain, for example a region of the lambda 6 light chain, or other lambda light chain peptides as an affinity reagent.

Human or humanized antibodies can be designed to have IgG, IgD, IgA and IgE constant region, and any isotype, including IgG1, IgG2, IgG3 and IgG4. Antibodies can be expressed as tetramers containing two light and two heavy chains, as separate heavy chains, light chains, as Fab, Fab′F(ab′)₂, and Fv, or as single chain antibodies in which heavy and light chain variable domains are linked through a spacer.

a. Production of Non-Human Antibodies. The production of non-human monoclonal antibodies, e.g., murine, guinea pig, rabbit or rat, can be accomplished by, for example, immunizing the animal with an immunogenic peptide, for example but not limited to a peptide with any of LFA-1 (αLβ2), Mac-1 (αMβ2), p150.95 (αXβ2), αDβ2, VLA-4 (α4β1), and β₇ (α4β7 and αEβ7) or homologues thereof. Any immunogenic peptide substantially similar to a region of the any of the following: LFA-1 (αLβ2), Mac-1 (αMβ2), p150.95 (αXβ2), αDβ2, VLA-4 (α4β1), and β₇ (α4β7 and αEβ7) or homologues thereof are encompassed for use See e.g., Harlow Lane, Antibodies, A Laboratory Manual (CSHP NY, 1988) (incorporated by reference for all purposes). Such immunogenic peptides can be obtained from a natural source, by peptide synthesis or by recombinant expression. Optionally, immunogenic peptides can be administered fused or otherwise complexed with a carrier protein, as described herein. Optionally, immunogenic peptides can be administered with an adjuvant. Several types of adjuvant can be used as described herein. Complete Freund's adjuvant followed by incomplete adjuvant is preferred for immunization of laboratory animals. Rabbits or guinea pigs are typically used for making polyclonal antibodies. Mice are typically used for making monoclonal antibodies. Antibodies are screened for specific binding to the immunogen. Optionally, antibodies are further screened for binding to a specific region of the immunogen, for example the lambda light chain of an immunoglobulin. Binding can be assessed, for example, by Western blot or ELISA. The smallest fragment to show specific binding to the antibody defines the epitope of the antibody. Alternatively, epitope specificity can be determined by a competition assay is which a test and reference antibody compete for binding to the component. If the test and reference antibodies compete, then they bind to the same epitope or epitopes sufficiently proximal that binding of one antibody interferes with binding of the other.

b. Chimeric and Humanized Antibodies. Chimeric and humanized antibodies have the same or similar binding specificity and affinity as a mouse or other nonhuman antibody that provides the starting material for construction of a chimeric or humanized antibody. Chimeric antibodies are antibodies whose light and heavy chain genes have been constructed, typically by genetic engineering, from immunoglobulin gene segments belonging to different species. For example, the variable (V) segments of the genes from a mouse monoclonal antibody may be joined to human constant (C) segments, such as IgG1 and IgG4. A typical chimeric antibody is thus a hybrid protein consisting of the V or antigen-binding domain from a mouse antibody and the C or effector domain from a human antibody.

Humanized antibodies have variable region framework residues substantially from a human antibody (termed an acceptor antibody) and complementarity determining regions substantially from a mouse-antibody, (referred to as the donor immunoglobulin). See, Queen et al., Proc. Natl. Acad. Sci. USA 86:10029-10033 (1989) and WO 90/07861, U.S. Pat. No. 5,693,762, U.S. Pat. No. 5,693,761, U.S. Pat. No. 5,585,089, U.S. Pat. No. 5,530,101 and Winter, U.S. Pat. No. 5,225,539 (incorporated by reference in their entirety for all purposes). The constant region(s), if present, are also substantially or entirely from a human immunoglobulin. The human variable domains are usually chosen from human antibodies whose framework sequences exhibit a high degree of sequence identity with the murine variable region domains from which the CDRs were derived. The heavy and light chain variable region framework residues can be substantially similar to a region of the same or different human antibody sequences. The human antibody sequences can be the sequences of naturally occurring human antibodies or can be consensus sequences of several human antibodies. See Carter et al., WO 92/22653. Certain amino acids from the human variable region framework residues are selected for substitution based on their possible influence on CDR conformation and/or binding to antigen. Investigation of such possible influences is by modeling, examination of the characteristics of the amino acids at particular locations, or empirical observation of the effects of substitution or mutagenesis of particular amino acids.

For example, when an amino acid differs between a murine variable region framework residue and a selected human variable region framework residue, the human framework amino acid should usually be substituted by the equivalent framework amino acid from the mouse antibody when it is reasonably expected that the amino acid: (1) non-covalently binds antigen directly, (2) is adjacent to a CDR region, (3) otherwise interacts with a CDR region (e.g. is within about 6 A of a CDR region), or (4) participates in the VL-VH interface.

Other candidates for substitution are acceptor human framework amino acids that are unusual for a human immunoglobulin at that position. These amino acids can be substituted with amino acids from the equivalent position of the mouse donor antibody or from the equivalent positions of more typical human immunoglobulins. Other candidates for substitution are acceptor human framework amino acids that are unusual for a human immunoglobulin at that position. The variable region frameworks of humanized immunoglobulins usually show at least 85% sequence identity to a human variable region framework sequence or consensus of such sequences.

c. Human Antibodies. Human antibodies against Ax3b2 are provided by a variety of techniques described below. Some human antibodies are selected by competitive binding experiments, or otherwise, to have the same epitope specificity as a particular mouse antibody. Human antibodies can also be screened for a particular epitope specificity by using only an immunogenic peptides of the present invention as the immunogen, and/or by screening antibodies for ability to kill plasma cells, as described in the examples.

(1) Trioma Methodology. The basic approach and an exemplary cell fusion partner, SPAZ-4, for use in this approach have been described by Oestberg et al., Hybridoma 2:361-367 (1983); Oestberg, U.S. Pat. No. 4,634,664; and Engleman et al., U.S. Pat. No. 4,634,666 (each of which is incorporated by reference in its entirety for all purposes). The antibody-producing cell lines obtained by this method are called triomas, because they are descended from three cells—two human and one mouse. Initially, a mouse multiple myeloma line is fused with a human B-lymphocyte to obtain a non-antibody-producing xenogeneic hybrid cell, such as the SPAZ-4 cell line described by Oestberg, supra. The xenogeneic cell is then fused with an immunized human B-lymphocyte to obtain an antibody-producing trioma cell line. Triomas have been found to produce antibody more stably than ordinary hybridomas made from human cells.

The immunized B-lymphocytes are obtained from the blood, spleen, lymph nodes or bone marrow of a human donor. If antibodies against a specific antigen or epitope are desired, it is preferable to use that antigen or epitope thereof for immunization. Immunization can be either in vivo or in vitro. For in vivo immunization, B cells are typically isolated from a human immunized with an immunogenic peptide, for example proteins or fragments thereof of LFA-1 (αLβ2), Mac-1 (αMβ2), p150.95 (αXβ2), VLA-4 (α4β1), and β₇ (α4β7 and αEβ7) or homologues thereof. For in vitro immunization, B-lymphocytes are typically exposed to antigen for a period of 7-14 days in a media such as RPMI-1640 (see Engleman, supra) supplemented with 10% human plasma.

The immunized B-lymphocytes are fused to a xenogeneic hybrid cell such as SPAZ-4 by well known methods. For example, the cells are treated with 40-50% polyethylene glycol of MW 1000-4000, at about 37 degrees C., for about 5-10 min. Cells are separated from the fusion mixture and propagated in media selective for the desired hybrids (e.g., HAT or AH). Clones secreting antibodies having the required binding specificity are identified by assaying the trioma culture medium for the ability to bind to an integrin as disclosed herein, such as proteins LFA-1 (αLβ2), Mac-1 (αMβ2), p150.95 (αXβ2), αDβ2, VLA-4 (α4β1), and β₇ (α4β7 and αEβ7) or homologues thereof. Triomas producing human antibodies having the desired specificity are subcloned by the limiting dilution technique and grown in vitro in culture medium.

Although triomas are genetically stable they do not produce antibodies at very high levels. Expression levels can be increased by cloning antibody genes from the trioma into one or more expression vectors, and transforming the vector into standard mammalian, bacterial or yeast cell lines, according to methods well known in the art.

(2) Transgenic Non-Human Mammals. Human antibodies against immunoglobulin light chains can also be produced from non-human transgenic mammals having transgenes encoding at least a segment of the human immunoglobulin locus. Usually, the endogenous immunoglobulin locus of such transgenic mammals is functionally inactivated. Preferably, the segment of the human immunoglobulin locus includes unrearranged sequences of heavy and light chain components. Both inactivation of endogenous immunoglobulin genes and introduction of exogenous immunoglobulin genes can be achieved by targeted homologous recombination, or by introduction of YAC chromosomes. The transgenic mammals resulting from this process are capable of functionally rearranging the immunoglobulin component sequences, and expressing a repertoire of antibodies of various isotypes encoded by human immunoglobulin genes, without expressing endogenous immunoglobulin genes. The production and properties of mammals having these properties are described in detail by, e.g., Lonberg et al., WO93/12227 (1993); U.S. Pat. No. 5,877,397, U.S. Pat. No. 5,874,299, U.S. Pat. No. 5,814,318, U.S. Pat. No. 5,789,650, U.S. Pat. No. 5,770,429, U.S. Pat. No. 5,661,016, U.S. Pat. No. 5,633,425, U.S. Pat. No. 5,625,126, U.S. Pat. No. 5,569,825, U.S. Pat. No. 5,545,806, Nature 148, 1547-1553 (1994), Nature Biotechnology 14, 826 (1996), Kucherlapati, WO 91/10741 (1991) (each of which is incorporated by reference in its entirety for all purposes). Transgenic mice are particularly suitable in this regard. Monoclonal antibodies are prepared by, e.g., fusing B-cells from such mammals to suitable multiple myeloma cell lines using conventional Kohler-Milstein technology. Human polyclonal antibodies can also be provided in the form of serum from humans immunized with an immunogenic agent.

(3) Phage Display Methods. A further approach for obtaining anti-immunglobulin light chains antibodies, for example anti-lambda6 containing immunoglobulin antibodies is to screen a DNA library front human B cells according to the general protocol outlined by Huse et al., Science 246:1275-1281 (1989). For example, as described for trioma methodology, such B cells can be obtained from a human immunized with an immunogenic peptide, for example integrin as disclosed herein such as proteins LFA-1 (αLβ2), Mac-1 (αMβ2), p150.95 (αXβ2), αDβ2, VLA-4 (α4β1), and β₇ (α4β7 and αEβ7) or homologues thereof. Optionally, such B cells are obtained from a patient who is ultimately to receive antibody treatment. Sequences encoding such antibodies (or binding fragments) are then cloned and amplified. The protocol described by Huse is rendered more efficient in combination with phage-display technology. See, e.g., Dower et al., WO 91/17271 and McCafferty et al., WO 92/01047, U.S. Pat. No. 5,877,218, U.S. Pat. No. 5,871,907, U.S. Pat. No. 5,858,657, U.S. Pat. No. 5,837,242, U.S. Pat. No. 5,733,743 and U.S. Pat. No. 5,565,332 (each of which is incorporated by reference in its entirety for all purposes). In these methods, libraries of phage are produced in which members display different antibodies on their outer surfaces. Antibodies are usually displayed as Fv or Fab fragments. Phage displaying antibodies with a desired specificity are selected by affinity enrichment to proteins; LFA-1 (αLβ2), Mac-1 (αMβ2), p150.95 (αXβ2), VLA-4 (α4β1), and β₇ (α4β7 and αEβ7) or homologues thereof.

In a variation of the phage-display method, human antibodies having the binding specificity of a selected murine antibody can be produced. See Winter, WO 92/20791. In this method, either the heavy or light chain variable region of the selected murine antibody is used as a starting material. If, for example, a light chain variable region is selected as the starting material, a phage library is constructed in which members display the same light chain variable region (i.e., the murine starting material) and a different heavy chain variable region. The heavy chain variable regions are obtained from a library of rearranged human heavy chain variable regions. A phage showing strong specific binding for the component of interest (e.g., at least 10⁸ and preferably at least 10⁹ M⁻¹) is selected. The human heavy chain variable region from this phage then serves as a starting material for constructing a further phage library. In this library, each phage displays the same heavy chain variable region (i.e., the region identified from the first display library) and a different light chain variable region. The light chain variable regions are obtained from a library of rearranged human variable light chain regions. Again, phage showing strong specific binding for amyloid peptide component are selected. These phage display the variable regions of completely human anti-amyloid peptide antibodies. These antibodies usually have the same or similar epitope specificity as the murine starting material.

d. Selection of Constant Region. The heavy and light chain variable regions of chimeric, humanized, or human antibodies can be linked to at least a portion of a human constant region. The choice of constant region depends, in part, whether antibody-dependent complement and/or cellular mediated toxicity is desired. For example, isotopes IgG1 and IgG3 have complement activity and isotypes IgG2 and IgG4 do not. Choice of isotype can also affect passage of antibody into the brain. Light chain constant regions can be lambda or kappa. Antibodies can be expressed as tetramers containing two light and two heavy chains, as separate heavy chains, light chains, as Fab, Fab F(ab)², and Fv, or as single chain antibodies in which heavy and light chain variable domains are linked through a spacer.

e. Expression of Recombinant Antibodies. Chimeric, humanized and human antibodies are typically produced by recombinant expression. Recombinant polynucleotide constructs typically include an expression control sequence operably linked to the coding sequences of antibody chains, including naturally-associated or heterologous promoter regions. Preferably, the expression control sequences are eukaryotic promoter systems in vectors capable of transforming or transfecting eukaryotic host cells. Once the vector has been incorporated into the appropriate host, the host is maintained under conditions suitable for high level expression of the nucleotide sequences, and the collection and purification of the cross-reacting antibodies.

These expression vectors are typically replicable in the host organisms either as episomes or as an integral part of the host chromosomal DNA. Commonly, expression vectors contain selection markers, e.g., ampicillin-resistance or hygromycin-resistance, to permit detection of those cells transformed with the desired DNA sequences.

E. coli is one prokaryotic host particularly useful for cloning the DNA sequences of the present invention. Microbes, such as yeast are also useful for expression. Saccharomyces is a preferred yeast host, with suitable vectors having expression control sequences, an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for maltose and galactose utilization.

Mammalian cells are a preferred host for expressing nucleotide segments encoding immunoglobulins or fragments thereof. See Winnacker, From Genes to Clones, (VCH Publishers, NY, 1987). A number of suitable host cell lines capable of secreting intact heterologous proteins have been developed in the art, and include CHO cell lines, various COS cell lines, HeLa cells, L cells and multiple myeloma cell lines. Expression vectors for these cells can include expression control sequences, such as an origin of replication, a promoter, an enhancer (Queen et al., Immunol. Rev. 89:49 (1986)), and necessary processing information sites, such as ribosome binding sites, RNA splice sites, polyadenylation sites, and transcriptional terminator sequences. Preferred expression control sequences are promoters substantially similar to a region of the endogenous genes, cytomegalovirus, SV40, adenovirus, bovine papillomavirus, and the like. See Co et al., J. Immunol. 148:1149 (1992).

Alternatively, antibody coding sequences can be incorporated in transgenes for introduction into the genome of a transgenic animal and subsequent expression in the milk of the transgenic animal (e.g.; according to methods described in U.S. Pat. No. 5,741,957, U.S. Pat. No. 5,304,489, U.S. Pat. No. 5,849,992, all incorporated by reference herein in their entireties). Suitable transgenes include coding sequences for light and/or heavy chains in operable linkage with a promoter and enhancer from a mammary gland specific gene, such as casein or beta lactoglobulin.

The vectors containing the DNA segments of interest can be transferred into the host cell by well-known methods, depending on the type of cellular host. For example, calcium chloride transfection is commonly utilized for prokaryotic cells, whereas calcium phosphate treatment, electroporation, lipofection, biolistics or viral-based transfection can be used for other cellular hosts. Other methods used to transform mammalian cells include the use of polybrene, protoplast fusion, liposomes, electroporation, and microinjection (see generally, Sambrook et al., supra). For production of transgenic animals, transgenes can be microinjected into fertilized oocytes, or can be incorporated into the genome of embryonic stem cells, and the nuclei of such cells transferred into enucleated oocytes.

Once expressed, antibodies can be purified according to standard procedures of the art, including HPLC purification, column chromatography, gel electrophoresis and the like (see generally, Scopes, Protein Purification (Springer-Verlag, NY, 1982)). The antibodies with affinity for a RCC biomarker protein as disclosed herein can be assessed by one of ordinary skill in the art, such as, for example but not limited to, western blot analysis on a purified RCC biomarker protein, ora biological sample comprising a RCC biomarker protein or fragment or variant thereof.

Detection of antibodies with affinity for an integrin or a cell surface marker expressed on a target cell can be achieved by direct labeling of the antibodies themselves, with labels including a radioactive label such as ³H, ¹⁴C, ³⁵S, ¹²⁵I, or ¹³¹I, a fluorescent label, a hapten label such as biotin, or an enzyme such as horse radish peroxidase or alkaline phosphatase. Alternatively, unlabeled primary antibody is used in conjunction with labeled secondary antibody, comprising antisera, polyclonal antisera or a monoclonal antibody specific for the primary antibody. In a preferred embodiment, the primary antibody or antisera is unlabeled, the secondary antisera or antibody is conjugated with biotin and enzyme-linked strepavidin is used to produce visible staining for histochemical analysis.

### Carrier Particles

One aspect of the present invention relates to compositions and methods for the delivery of at least one soluble agent and at least one insoluble agent to a target cell. As disclosed herein, the composition comprises a carrier particle comprising an insoluble agent and/or a soluble agent, wherein the carrier particle is attached to or conjugated to a targeting moiety, where the targeting moiety binds to (or has specific affinity for) to a cell surface marker on the target cell. As discussed above, in some embodiments depending on the target cell of the attached targeting moiety, the carrier particle is a component of a leukocyte delivery agent or an endothelial cell delivery agent.

In some embodiments, the carrier particles are micro-lipid particles or nano-lipid particles, e.g., liposomes, spheres, micelles. In some embodiments the carrier particles are unilammar, (meaning the carrier particles comprise more than one layer or are multi-layered). In some embodiments, a first layer contains agents that facilitate cryoprotection, long half-life in circulation, or both (PEG, hyaluronan, others).

Carrier particles as disclosed herein include any carrier particle modifiable by attachment of a targeting moiety known to the skilled artisan. Carrier particles include but are not limited to liposomal or polymeric nanoparticles such as liposomes, proteins, and non-protein polymers. Carrier particles can be selected according to (i) their ability to transport the agent of choice and (ii) the ability to associate with a targeting moiety as disclosed herein.

In some embodiments, carrier particles include colloidal dispersion systems, which include, but are not limited to, macromolecule complexes, nanocapsules, microspheres, beads and lipid-based systems including oil-in-water emulsions, micelles, mixed micelles, liposomes and lipid:oligonucleotide complexes of uncharacterized structure. In some embodiments, the carrier particle comprises a plurality of liposomes. Liposomes are microscopic spheres having an aqueous core surrounded by one or more outer layers made up of lipids arranged in a bilayer configuration (see, generally, Chonn et al., Current Op. Biotech. 1995, 6, 698-708). Other carrier particles are cellular uptake or membrane-disruption moieties, for example polyamines, e.g. spermidine or spermine groups, or polylysines; lipids and lipophilic groups; polymyxin or polymyxin-derived peptides; octapeptin; membrane pore-forming peptides; ionophores; protamine; aminoglycosides; polyenes; and the like. Other potentially useful functional groups include intercalating agents; radical generators; alkylating agents; detectable labels; chelators; or the like.

One can use other carrier particles, for example lipid particle or vesicle, such as a liposome or microcrystal, which may be suitable for parenteral administration. The particles may be of any suitable structure, such as unilamellar or plurilamellar, so long as the antisense oligonucleotide is contained therein. Positively charged lipids such as N—[I-(2,3dioleoyloxi)propyl]-N,N,N-trimethyl-anunoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757 which are incorporated herein by reference. Other non-toxic lipid based vehicle components may likewise be utilized to facilitate uptake of the antisense compound by the cell.

In some embodiments, a carrier particle is a liposome. Liposomes are completely closed lipid bilayer membranes containing an entrapped aqueous volume. Liposomes may be unilamellar vesicles possessing a single membrane bilayer or multilameller vesicles, onion-like structures characterized by multiple membrane bilayers, each separated from the next by an aqueous layer. In one preferred embodiment, the liposomes of the present invention are unilamellar vesicles. The bilayer is composed of two lipid monolayers having a hydrophobic “tail” region and a hydrophilic “head” region. The structure of the membrane bilayer is such that the hydrophobic (nonpolar) “tails” of the lipid monolayers orient toward the center of the bilayer while the hydrophilic “heads” orient towards the aqueous phase.

Liposomes useful in the methods and compositions as disclosed herein can be produced from combinations of lipid materials well known and routinely utilized in the art to produce liposomes. Lipids can include relatively rigid varieties, such as sphingomyelin, or fluid types, such as phospholipids having unsaturated acyl chains. “Phospholipid” refers to any one phospholipid or combination of phospholipids capable of forming liposomes. Phosphatidylcholines (PC), including those obtained from egg, soy beans or other plant sources or those that are partially or wholly synthetic, or of variable lipid chain length and unsaturation are suitable for use in the present invention.

Synthetic, semisynthetic and natural product phosphatidylcholines including, but not limited to, distearoylphosphatidylcholine (DSPC), hydrogenated soy phosphatidylcholine (HSPC), soy phosphatidylcholine (soy PC), egg phosphatidylcholine (egg PC), hydrogenated egg phosphatidylcholine (HEPC), dipahnitoylphosphatidylcholine (DPPC) and dimyristoylphosphatidylcholine (DMPC) are suitable phosphatidylcholines for use in this invention. All of these phospholipids are commercially available. Further, phosphatidylglycerols (PG) and phosphatic acid (PA) are also suitable phospholipids for use in the present invention and include, but are not limited to, dimyristoylphosphatidylglycerol (DMPG), dilaurylphosphatidylglycerol (DLPG), dipalmitoylphosphatidylglycerol (DPPG), distearoylphosphatidylglycerol (DSPG) dimyristoylphosphatidic acid (DMPA), distearoylphosphatidic acid (DSPA), dilaurylphosphatidic acid (DLPA), and dipalmitoylphosphatidic acid (DPPA). Distearoylphosphatidylglycerol (DSPG) is the preferred negatively charged lipid when used in formulations. Other suitable phospholipids include phosphatidylethanolamines, phosphatidylinositols, sphingomyelins, and phosphatidic acids containing lauric, myristic, stearoyl, and palmitic acid chains. For the purpose of stabilizing the lipid membrane, it is preferred to add an additional lipid component, such as cholesterol. Preferred lipids for producing liposomes according to the invention include phosphatidylethanolamine (PE) and phosphatidylcholine (PC) in further combination with cholesterol (CH). According to one embodiment of the invention, a combination of lipids and cholesterol for producing the liposomes of the invention comprise a PE:PC:Chol molar ratio of 3:1:1. Further, incorporation of polyethylene glycol (PEG) containing phospholipids is also contemplated by the present invention.

Liposomes useful in the methods and compositions as disclosed herein can be obtained by any method known to the skilled artisan. For example, the liposome preparation of the present invention can be produced by reverse phase evaporation (REV) method (see U.S. Pat. No. 4,235,871), infusion procedures, or detergent dilution. A review of these and other methods for producing liposomes can be found in the text Liposomes, Marc Ostro, ed., Marcel Dekker, Inc., New York, 1983, Chapter 1. See also Szoka Jr. et al., (1980, Ann. Rev. Biophys. Bioeng., 9:467). A method for forming ULVs is described in Cullis et al., PCT Publication No. 87/00238, Jan. 16, 1986, entitled “Extrusion Technique for Producing Unilamellar Vesicles”. Multilamellar liposomes (MLV) can be prepared by the lipid-film method, wherein the lipids are dissolved in a chloroform-methanol solution (3:1, vol/vol), evaporated to dryness under reduced pressure and hydrated by a swelling solution. Then, the solution is subjected to extensive agitation and incubation, e.g., 2 hour, e.g., at 37° C. After incubation, unilamellar liposomes (ULV) are obtained by extrusion. The extrusion step modifies liposomes by reducing the size of the liposomes to a preferred average diameter. Alternatively, liposomes of the desired size can be selected using techniques such as filtration or other size selection techniques. While the size-selected liposomes of the invention should have an average diameter of less than about 300 nm, it is preferred that they are selected to have an average diameter of less than about 200 nm with an average diameter of less than about 100 nm being particularly preferred. When the liposome of the present invention is a unilamellar liposome, it preferably is selected to have an average diameter of less than about 200 nm. The most preferred unilamellar liposomes of the invention have an average diameter of less than about 100 nm. It is understood, however, that multivesicular liposomes of the invention derived from smaller unilamellar liposomes will generally be larger and can have an average diameter of about less than 1000 nm. Preferred multivesicular liposomes of the invention have an average diameter of less than about 800 nm, and less than about 500 nm while most preferred multivesicular liposomes of the invention have an average diameter of less than about 300 nm.

In another embodiment, the carrier particle is a cyclodextrin-based nanoparticle. Polycation formulated nanoparticles have been used for drug delivery into the brain as well as for systemic delivery of siRNA. A unique cyclodextrin-based nanoparticle technology has been developed for targeted gene delivery in vivo. This delivery system consists of two components. The first component is a biologically non-toxic cyclodextrin-containing polycation (CDP). CDPs self-assemble with siRNA to form colloidal particles about 50 nm in diameter and protects si/shRNA against degradation in body fluids. Moreover, the CDP has been engineered to contain imidazole groups at their termini to assist in the intracellular trafficking and release of the nucleic acid. CDP also enables assembly with the second component. The second component is an adamantane-terminated polyethylene glycol (PEG) modifier for stabilizing the particles in order to minimize interactions with plasma and to increase the attachment to the cell surface targeting molecules such as integrins, such as disclosed herein LFA-1 (αLβ2), Mac-1 (αMβ2), p150.95 (αXβ2), αDβ2, VLA-4 (α4β1), and β₇ (α4β7 and αEβ7), for example in a leukocyte delivery agent or integrin ligands, such as ICAM-1, ICAM-2, ICAM-3, VCAM-1, MAdCAM-1, E-cadherin, JAM-1, JAM-2 and JAM-3 for example in endothelial delivery agent. Thus, the advantages of this delivery system are: 1) since the CDP protects the siRNA from degradation, chemical modification of the nucleic acid is unnecessary, 2) the colloidal particles do not aggregate and have extended life in biological fluids because of the surface decoration with PEG that occurs via inclusion complex formation between the terminal adamantane and the cyclodextrins, 3) cell type-specific targeted delivery is possible because some of the PEG chains contain targeting ligands, 4) it does not induce an immune response, and 5) in vivo delivery does not produce an interferon response even when a siRNA is used that contains a motif known to be immunostimulatory when delivered in vivo with lipids.

In another embodiment, the carrier particle is a cationic peptide, e.g., protamine. See, for example, WO 06/023491, which is specifically incorporated herein in its entirety by reference.

The glycosaminoglycan carrier particles disclosed in U.S. Patent Appl. No. 20040241248 and the glycoprotein carrier particles in WO 06/017195, which are incorporated herein in their entirety by reference, are useful in the methods of the present invention. Similar naturally occurring polymer-type carriers known to the skilled artisan are also useful in the methods of the present invention.

Soluble non-protein polymers are also useful as carrier particles. Such polymers can include polyvinylpyrrolidone, pyran copolymer, polyhydroxypropylrnethacrylamidephenol, polyhydroxyethylaspartamidephenol, or polyethyleneoxidepolylysine substituted with palitoyl residues. Furthermore, the therapeutic agents can be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates, and cross-linked or amphipathic block copolymers of hydrogels. The therapeutic agents can also be affixed to rigid polymers and other structures such as fullerenes or Buckeyballs.

Conjugation of a Targeting Moiety with a Carrier Particle

A carrier particle as disclosed herein can be associated with the targeting moiety. The association of a carrier particle with a targeting moiety can be a non-covalent or covalent interaction, for example, by means of chemical cross-linkage or conjugation. In the composition and methods disclosed herein, a targeting moiety is associated with a carrier particle, for example liposome.

As used herein, the term “associated with” means that one entity is in physical association or contact with another. Thus, a targeting moiety “associated with” a carrier particle can be either covalently or non-covalently joined to the carrier particle. The association can be mediated by a linker moiety, particularly where the association is covalent. The term “association” or “interaction” or “associated with” are used interchangeably herein and as used in reference to the association or interaction of a targeting moiety, e.g., an anti-integrin antibody of fragment thereof with a carrier particle for example liposome, refers to any association between the targeting moiety with the carrier particle, for example a liposome comprising a hydrophilic agent and/or a hydrophobic agent, either by a direct linkage or an indirect linkage.

An indirect linkage includes an association between a targeting moiety, e.g., an anti-integrin antibody of fragment thereof, with a carrier particle for example liposome, wherein the targeting moiety and the carrier particle are attached via a linker moiety, e.g., they are not directly linked. Linker moieties include, but are not limited to, chemical linker moieties. In some embodiments, a linker between a targeting moiety and the carrier particle is formed by reacting the polymer and a linker selected e.g., from the group consisting of p-nitrophenyl chloroformate, carbonyldiimidazole (CDI), carbonate (DSC), cis-aconitic anhydride, and a mixture of these compounds.

A direct linkage includes any linkage wherein a linker moiety is not required. In one embodiment, a direct linkage includes a chemical or a physical interaction wherein the two moieties, i.e. the targeting moiety and carrier particle interact such that they are attracted to each other. Examples of direct interactions include covalent interactions, non-covalent interactions, hydrophobic/hydrophilic, ionic (e.g., electrostatic, coulombic attraction, ion-dipole, charge-transfer), Van der Waals, or hydrogen bonding, and chemical bonding, including the formation of a covalent bond. Accordingly, in one embodiment, a targeting moiety, such as an anti-integrin antibody of fragment thereof and the carrier particle are not linked via a linker, e.g., they are directly linked. In a further embodiment, a targeting moiety and the carrier particle are electrostatically associated with each other.

As used herein, the term“conjugate” or “conjugation” refers to the attachment of two or more entities to form one entity. For example, the methods of the present invention provide conjugation of a targeting moiety of the present invention joined with another entity, for example a carrier particle, for example a liposome. The attachment can be by means of linkers, chemical modification, peptide linkers, chemical linkers, covalent or non-covalent bonds, or protein fusion or by any means known to one skilled in the art. The joining can be permanent or reversible. In some embodiments, several linkers can be included in order to take advantage of desired properties of each linker and each protein in the conjugate. Flexible linkers and linkers that increase the solubility of the conjugates are contemplated for use alone or with other linkers as disclosed herein. Peptide linkers can be linked by expressing DNA encoding the linker to one or more proteins in the conjugate. Linkers can be acid cleavable, photocleavable and heat sensitive linkers. Methods for conjugation are well known by persons skilled in the art and are encompassed for use in the present invention.

According to the present invention, the targeting moiety such as an antibody, antibody fragment, integrin or integrin ligand or fragments thereof, can be linked to the carrier particle entity via any suitable means, as known in the art, see for example U.S. Pat. Nos. 4,625,014, 5,057,301 and 5,514,363, which are incorporated herein in their entirety by reference. For example, the agent to be transported can be covalently conjugated to the carrier particle, either directly or through one or more linkers. In one embodiment, the carrier particle of the present invention is conjugated directly to an agent to be transported. In another embodiment, the carrier particle of the present invention is conjugated to an agent to be transported to leukocytes via a linker, e.g. a transport enhancing linker.

A large variety of methods for conjugation of targeting moiety with carrier particles are known in the art. Such methods are e.g. described by Hermanson (1996, Bioconjugate Techniques, Academic Press), in U.S. Pat. No. 6,180,084 and U.S. Pat. No. 6,264,914 which are incorporated herein in their entirety by reference and include e.g. methods used to link haptens to carriers proteins as routinely used in applied immunology (see Harlow and Lane, 1988, “Antibodies: A laboratory manual”, Cold Spring Harbor Laboratory Press, Cold Spring Harbor, N.Y.). It is recognized that, in some cases, a targeting moiety or carrier particle can lose efficacy or functionality upon conjugation depending, e.g., on the conjugation procedure or the chemical group utilised therein. However, given the large variety of methods for conjugation the skilled person is able to find a conjugation method that does not or least affects the efficacy or functionality of the entities to be conjugated.

In some embodiments, the outer surface of the liposomes can be modified with a long-circulating agent, e.g., PEG, e.g., hyaluronic acid (HA). The liposomes can be modified with a cryoprotectant, e.g., a sugar, such as trehalose, sucrose, mannose or glucose, e.g., HA. In some embodiments, a liposome is coated with HA. HA acts as both a long-circulating agent and a cryoprotectant. The liposome is modified by attachment of the targeting moiety. In another embodiment, the targeting moiety is covalently attached to HA, which is bound to the liposome surface. Alternatively, a carrier particle is a micelle. Alternatively, the micelle is modified with a cryoprotectant, e.g., HA, PEG.

A method for coating the liposomes or other polymeric nanoparticles with a targeting moiety, such as an antibody or protein or peptide (such as of an integrin or integrin ligand or variants, derivatives or fragments thereof) are disclosed in U.S. Provisional Application No. 60/794,361 filed Apr. 24, 2006, and International Patent Application: PCT/US07/10075 filed Apr. 24, 2007 with are incorporated in their entirety herein by reference.

In some embodiments, the outer surface of the liposomes can be further modified with a long-circulating agent. The modification of the liposomes with a hydrophilic polymer as the long-circulating agent is known to enable to prolong the half-life of the liposomes in the blood. Examples of the hydrophilic polymer include polyethylene glycol, polymethylethylene glycol, polyhydroxypropylene glycol, polypropylene glycol, polymethylpropylene glycol and polyhydroxypropylene oxide. In one embodiment, a hydrophilic polymer is polyethylene glycol (PEG). Glycosaminoglycans, e.g., hyaluronic acid, can also be used as long-circulating agents.

In some embodiments, a targeting moiety, such as an antibody or protein or peptide (such as of an integrin or integrin ligand or variants, derivatives or fragments thereof) can be conjugated to a cryoprotectant present on the liposome, e.g., HA. Crosslinking reagents include glutaraldehyde (GAD), bifunctional oxirane (OXR), ethylene glycol diglycidyl ether (EGDE), N-hydroxysuccinimide (NHS), and a water soluble carbodiimide, preferably 1-ethyl-3-(3-dimethylaminopropyl)carbodiimide (EDC). As is known to the skilled artisan, any crosslinking chemistry can be used, including, but not limited to, thioether, thioester, malimide and thiol, amine-carboxyl, amine-amine, and others listed in organic chemistry manuals, such as, Elements of Organic Chemistry, Isaak and Henry Zimmerman Macmillan Publishing Co., Inc. 866 Third Avenue, New York, N.Y. 10022. Through the complex chemistry of crosslinking, linkage of the amine residues of the recognizing substance and liposomes is established.

In some embodiments, after a targeting moiety is associated with (e.g. conjugated or covalently attached) to the carrier particle by way of covalent linkage to the cryoprotectant, or by way of covalent linkage to another targeting moiety covalently linked to the cryoprotectant, the lipid particle may be lyophilized. The lipid particle may remain lyophilized prior to rehydration, or prior to rehydration and encapsulation of the agent of interest, for extended periods of time. In one embodiment, the lipid particle remains lyophilized for about 1 month, about 2 months, about 3 months, about 6 months, about 9 months, about 12 months, about 18 months, about 2 years or more prior to rehydration.

The term “cryoprotectant” as used herein refers to an agent that protects a lipid particle subjected to dehydration-rehydration, freeze-thawing, or lyophilization-rehydration from vesicle fusion and/or leakage of vesicle contents. Useful cryoprotectants in the methods of the present invention include hyaluronan/hyaluronic acid (HA) or other glycosaminoglycans for use with liposomes or micelles or PEG for use with micelles. Other cryoprotectants, but are not limited to, include disaccharide and monosaccharide sugars such as trehalose, maltose, sucrose, maltose, fructose, glucose, lactose, saccharose, galactose, mannose, xylit and sorbit, mannitol, dextran; polyols such as glycerol, glycerin, polyglycerin, ethylene glycol, prolylene glycol, polyethyleneglycol and branched polymers thereof; aminoglycosides; and dimethylsulfoxide.

In some embodiments, a liposome can be with a cryoprotectant. One preferred cryoprotectant of the present invention is hyaluronic acid or hyaluran (HA). Hyaluronic acid, a type of glycosaminoglycan, is a natural polymer with alternating units of N-acetyl glucosamine and glucoronic acid. Using a crosslinking reagent, hyaluronic acid offers carboxylic acid residues as functional groups for covalent binding. The N-acetyl-glucosamine contains hydroxyl units of the type —CH₂—OH which can be oxidized to aldehydes, thereby offering an additional method of crosslinking hyaluronic acid to the liposomal surface in the absence of a crosslinking reagent. Alternatively, other glycosaminoglycans, e.g., chondroitin sulfate, dermatan sulfate, keratin sulfate, or heparin, may be utilized in the methods of the present invention. Cryoprotectants are bound covalently to discrete sites on the liposome surfaces. The number and surface density of these sites will be dictated by the liposome formulation and the liposome type.

In one embodiment, the final ratio of cryoprotectant (μg) to lipid (μmole) is about 50 μg/μmole, about 55 μg/μmole, about 60 μg/μmole, about 65 μg/μmole, about 70 μg/μmole, about 75 μg/μmole, about 80 μg/μ mole, about 85 μg/μmole, about 90 μg/μmole, about 95 μg/μmole, about 100 μg/μmole, about 105 μg/μmole, about 120 μg/mole, about 150 μg/mole, or about 200 μg/mole. In one embodiment, the ratio of cryoprotectant (μg) to lipid (μmole) is a range from 3-200 μg per mole lipid.

To form covalent conjugates of cryoprotectants and liposomes, crosslinking reagents have been studied for effectiveness and biocompatibility. Crosslinking reagents include glutaraldehyde (GAD), bifunctional oxirane (OXR), ethylene glycol diglycidyl ether (EGDE), and a water soluble carbodiimide, preferably 1-ethyl-3-(3-dimethylaminopropyl)carbodiimide (EDC). Through the chemistry of crosslinking, linkage of the amine residues of the recognizing substance and liposomes is established. Covalent attachment of the cryoprotectant HA is described in U.S. Pat. No. 5,846,561.

Subsequent to the covalent addition of the cryoprotectant, the lipid particles may be lyophilized. The lyophilized lipid particles may be rehydrated and the targeting moiety (layer 2) covalently attached to the lipid particle. Alternatively, the targeting moiety may be covalently attached to the lipid particle without prior lyophilization and rehydration.

In some embodiments, the carrier particles are coated with a second layer containing targeting moieties, e.g., specific monoclonal antibodies, scFvs, Fab fragments, or receptor ligands. In such embodiments virtually any agent or drug can be encapsulated in the carriers via lyophilization and reconstitution with an agent suspended in aqueous solution.

In one embodiment, the invention provides a method of coating a lipid particle that is pre-conjugated with a cryoprotectant, wherein the cryoprotectant has a functional group attached. The attached functional group may be activated and a targeting moiety is crosslinked to the activated functional group to form a two-layer coated lipid particle which can then be lyophilized for storage purposes prior to use for drug or agent encapsulation.

In one embodiment, the invention is directed to a method to generate immunoliposomes for targeting leukocytes, comprising a composition which comprises a targeting moiety for targeted delivery to leukocytes and a carrier particle associated with the targeting moiety, wherein the carrier particle comprises at least one agent.

In one embodiment, the invention provides liposomes that may be stored in a lyophilized condition prior to encapsulation of drug or prior to addition of the targeting moiety.

Suitable methods for conjugation of a targeting moiety with carrier particle include e.g. carbodimide conjugation (Bauminger and Wilchek, 1980, Meth. Enzymol. 70: 151-159). Alternatively, a molecule can be coupled to a targeting moiety as described by Nagy et al., Proc. Natl. Acad. Sci. USA 93:7269-7273 (1996), and Nagy et al., Proc. Natl. Acad. Sci. USA 95:1794-1799 (1998), each of which are incorporated herein by reference. Another method for conjugating one can use is, for example sodium periodate oxidation followed by reductive alkylation of appropriate reactants and glutaraldehyde crosslinking.

One can use a variety of different linkers to conjugate the targeting moiety, for example antibody, antibody fragment, integrin ligand or integrin ligand fragment to a carrier particle, for example but not limited to aminocaproic horse radish peroxidase (HRP) or a heterobiofunctional cross-linker, e.g. carbonyl reactive and sulfhydryl-reactive cross-linker. Heterobiofunctional cross linking reagents usually contain two reactive groups that can be coupled to two different function targets on proteins and other macromolecules in a two or three-step process, which can limit the degree of polymerization often associated with using homobiofunctional cross-linkers. Such multistep protocols can offer a great control of conjugate size and the molar ratio of components.

The term “linker” refers to any means to join two or more entities, for example a peptide with another peptide, or a liposome. A linker can be a covalent linker or a non-covalent linker. Examples of covalent linkers include covalent bonds or a linker moiety covalently attached to one or more of the proteins to be linked. The linker can also be a non-covalent bond, e.g. an organometallic bond through a metal center such as platinum atom. For covalent linkages, various functionalities can be used, such as amide groups, including carbonic acid derivatives, ethers, esters, including organic and inorganic esters, amino, urethane, urea and the like. To provide for linking, the effector molecule and/or the probe can be modified by oxidation, hydroxylation, substitution, reduction etc. to provide a site for coupling. It will be appreciated that modification which do not significantly decrease the function of the target moiety, for example antibody, antibody fragment, integrin ligand or integrin ligand fragment and/or the carrier particle are preferred.

In some embodiments where the carrier particle is a liposome or polymeric nanoparticle, a targeting moiety, such as for example antibody, antibody fragment, integrin ligand or integrin ligand fragment is captured within the carrier particle, for example liposomes or polymeric nanoparticle. For example, a suspension of an antibody, antibody fragment, integrin ligand or integrin ligand fragment or variant thereof can be encapsulated in micelles to form liposomes by conventional methods (U.S. Pat. No. 5,043,164, U.S. Pat. No. 4,957,735, I5 U.S. Pat. No. 4,925,661; Connor and Huang, (1985) J. Cell Biol. 101: 581; Lasic D. D. (1992) Nature 355: 279; Novel Drug Delivery (eds. Prescott and Nimmo, Wiley, New York, 1989); Reddy et al. (1992) J. Immunol. 148:1585), which are incorporated herein in their entirety by reference. Liposomes comprising targeting moiety that binds specifically to leukocytes expressing, for example at least one integrin selected from, LFA-1 (αLβ2), Mac-1 (αMβ2), p150.95 (αXβ2), αDβ2, VLA-4 (α4β1), and β₇ (α4β7 and αEβ7) can be used to target the agents to those cells.

In some embodiments, and in the event that the carrier particle is a peptide or protein, and the targeting moiety is also a peptide or antibody, or contains amino acids as part of its structure, the targeting moiety (for example an antibody or integrin or integrin ligand, of fragments thereof) can be fused either in frame or out of frame with the carrier particle to form a fusion protein. In general, the targeting moiety (i.e. an antibody or protein or fragment of an integrin or integrin ligand) and carrier particle can be fused directly or via one or more amino acid linkers. Any suitable amino acid linkers can be used to modify the stability, conformation, charge, or other structure features of the resulting fusion protein in order to facilitate its transport to target cells. In some embodiments, fusion proteins can also be formed from the carrier particle and agent, where both the carrier particle and agents are proteins or contain amino acids as part of their structure, and preferably the activity of the agent is not compromised by being fused with the carrier particle. The term “fusion protein” refers to a recombinant protein of two or more fused proteins.

Fusion proteins can be produced, for example, by a nucleic acid sequence encoding one protein joined to the nucleic acid encoding another protein such that they constitute a single open-reading frame that can be translated in the cells into a single polypeptide harboring all the intended proteins. The order of arrangement of the proteins can vary. As a non-limiting example, a nucleic acid sequence encoding an integrin ligand, as a non-limiting example, a nucleic acid encoding ICAM-1 can be fused to either the 5′ or the 3′ end of the nucleic acid sequence encoding a carrier particle. In this manner, on expression of the nucleic acid construct, the ICAM-1 or fragment thereof is functionally expressed and fused to the N-terminal or C-terminal end of the carrier protein. In certain embodiments, the carrier peptide can be modified such that the carrier protein function (i.e. ability to associate with the agent) remains unaffected by fusion to the targeting moiety and vice versa, the targeting moiety can be modified, for example the ICAM-1 protein and/or fragment thereof can be used so that the ICAM-1 retains the ability to bind to its integrin, for example LFA-1 even when fused with another protein, for example the carrier particle.

In some embodiments, the leukocyte delivery agent can comprise a liposomes comprising multiple layers that assembled in a step-wise fashion, where each layer can comprise a targeting moiety. In one embodiment, the first step is the preparation of empty nano-scale liposomes. Liposomes may be prepared by any method known to the skilled artisan. The second step is the addition of a first layer of surface modification. The first layer is added to the liposome by covalent modification. The first layer comprises hyaluronic acid, or other cryoprotectant glucosaminoglycan. The liposome composition may also be lyophilized and reconstituted at any time after the addition of the first layer. The third step is to add a second surface modification. The second layer is added by covalent attachment to the first layer. The second layer comprises a targeting moiety, e.g., an antibody or functional fragment thereof. Further layers may add to the liposome and these layers may include additional targeting moieties. Alternatively, the second layer may include a heterogeneous mix of targeting moieties. The liposome composition is lyophilized after addition of the final targeting layer. An agent of interest is encapsulated by the liposome by rehydration of the liposome with an aqueous solution containing the agent. In one embodiment, agents that are poorly soluble in aqueous solutions or agents that are hydrophobic may be added to the composition during preparation of the liposomes in step one.

In another embodiment, a leukocyte delivery agent as disclosed herein can comprise a multi-layered liposome with cryoprotectant conjugated lipid particles. In such embodiments, a cryoprotectant can be covalently linked to the lipid polar groups of the phospholipids and it forms the first layer of surface modification on the liposome discussed supra. The targeting moiety forms the second layer of coat and it is added on to the first layer of cryoprotectant. The multi-layered liposome may be lyophilized for storage. The agent of interest is encapsulated by the liposome by rehydration of the liposome with an aqueous solution containing the agent.

### Agents

One aspect of the present invention relates to a composition for the simultaneous delivery of an insoluble agent and a soluble agent to a target cell, wherein the composition comprises a carrier particle comprising an insoluble agent and/or a soluble agent, wherein the carrier particle is attached or conjugated to a targeting moiety, where the targeting moiety binds to and has specific affinity for to a cell surface marker on the target cell (i.e. the targeting moiety selectively targets the target cell).

In one embodiment, the invention is directed to leukocyte-selective delivery agents for delivery of at least two agents to a leukocyte. Methods to generate such leukocyte-selective delivery agents loaded with two agents are disclosed herein. In one embodiment, one of the two agents is hydrophilic (i.e. a soluble agent) which is entrapped the aqueous phase of the carrier particle, such as the center of a liposome. In another embodiment, the other agent is hydrophobic (or an insoluble agent) which is entrapped in the lipid phase of the carrier particle, for example a hydrophobic agent can be associated with a lipid layer of the liposome.

For purposes of the present invention, “agent” means any agent or compound that can affect the body therapeutically, or which can be used in vivo for diagnosis. Examples of therapeutic agents include chemotherapeutics for cancer treatment, antibiotics for treating infections, anti-fungals for treating fungal infections, therapeutic nucleic acids including nucleic acid analogs, e.g., siRNA.

An “agent” as used herein refers to an agent that is transported by the carrier particle and targeting moiety (i.e. an antibody to an integrin or an integrin ligand) to target the leukocyte. An agent can be a chemical molecule of synthetic or biological origin. In some embodiments, an agent is generally a molecule that can be used in a pharmaceutical composition, for example the agent is a therapeutic agent. An agent as used herein also refers to any chemical entity or biological product, or combination of chemical entities or biological products, administered to a subject to treat or prevent or control a disease or condition, and are herein referred to as “therapeutic agents”.

In alternative embodiments, an agent can be a chemical entity or biological product, or combination of chemical entities or biological products, administered to a subject for imaging purposes in the subject, for example to monitor the presence or progression of disease or condition, and are herein referred to as “imaging agents” or “diagnostic agents”.

A chemical entity or biological product as disclosed herein is preferably, but not necessarily a low molecular weight compound, but can also be a larger compound, or any organic or inorganic molecule, including modified and unmodified nucleic acids such as antisense nucleic acids, RNAi, such as siRNA, shRNA, miRNA, nucleic acid analogues, miRNA analogues, antigomirs, peptides, peptidomimetics, avimers, receptors, ligands, and antibodies, aptamers, polypeptides or analogues, derivatives or variants thereof. For example, oligomers of nucleic acids, amino acids, carbohydrates include without limitation proteins, oligonucleotides, ribozymes, DNAzymes, glycoproteins, siRNAs, lipoproteins, aptamers, and modifications, derivatives and combinations thereof.

A therapeutic agent is an agent useful in the treatment of a disease, disorder or malignancy. In some embodiments, the disease, disorder or malignancy is a disease with dysregulation of leukocytes and/or endothelial cells, and in some instances the disease results in accumulation of leukocytes, platelets, extravasation of leukocytes and increased vascular permeability at a site of tissue damage or sustained inflammation. Such diseases, disorders or malignancies include, for example but not limited to inflammatory disease, autoimmune disease, atherosclerosis, angiogenesis or ischemia-reperfusion injury. In some embodiment, the disease or disorder is associated with dysregulation of any cell expressing at least one integrin selected from the group of LFA-1 (αLβ2), Mac-1 (αMβ2), p150.95 (αXβ2), αDβ2, VLA-4 (α4β1), and β₇ (α4β7 and αEβ7).

Hydrophilic Agents or Soluble Agents

One aspect of the invention relates to compositions and methods to simultaneously deliver at least one insoluble agent and at least one soluble agent to a target cell, such as or example a lymphocyte or an endothelial cell. A soluble agent is also referred to as a water-soluble agent, a hydrophilic agent as that term is defined herein.

Any soluble agent is contemplated for delivery to a target cell using the methods and compositions as disclosed herein. Examples of soluble agents include, for example, but are not limited to, proteins, peptides, antibodies, antibody fragments, nucleic acids such as DNA and RNA and RNAi agents such as siRNA, miRNA and the like; nucleic acid analogs such PNA (peptide nucleic acid), LNA (locked nucleic acid), pcPNA (pseudo-complementary PNA) and the like, as other agents which are soluble as according to the term as defined herein. Typically, all globular proteins are soluble, which includes enzymes, enzyme fragments, and recombinant proteins. In some embodiments, a soluble protein useful for delivery using the compositions and methods as disclosed herein is a recombinant version or variant of a native protein which has been modified to increase its solubility and/or stability in solution. A soluble protein as disclosed herein is a protein which goes into solution. Stated another way, if 30% of a crude protein preparation (containing multiple proteins) goes into solution, 30% of the crude protein preparation comprises soluble proteins.

In some embodiments, an agent is a gene or polynucleotide, such as plasmid DNA, DNA fragment, oligonucleotide, oligodeoxynucleotide, antisense oligonucleotide, chimeric RNA/DNA oligonucleotide, RNA, siRNA, ribozyme, or viral particle.

In some embodiments, an agent is a nucleic acid, e.g., DNA, RNA, siRNA, plasmid DNA, short-hairpin RNA, small temporal RNA (stRNA), microRNA (miRNA), RNA mimetics, or heterochromatic siRNA. The nucleic acid agent of interest has a charged backbone that prevents efficient encapsulation in the lipid particle. Accordingly, the nucleic acid agent of interest may be condensed with a cationic polymer, e.g., PEI, polyamine spermidine, and spermine, or cationic peptide, e.g., protamine and polylysine, prior to encapsulation in the lipid particle. In one embodiment, the agent is not condensed with a cationic polymer.

In some embodiments, an agent functions as an RNA interference molecule. The term “RNAi” as used herein refers to interfering RNA, or RNA interference molecules are nucleic acid molecules or analogues thereof for example RNA-based molecules that inhibit gene expression. RNAi refers to a means of selective post-transcriptional gene silencing. RNAi can result in the destruction of specific mRNA, or prevents the processing or translation of RNA, such as mRNA.

In some embodiments, an agent is a siRNA. The term “short interfering RNA” (siRNA), also referred to herein as “small interfering RNA” is defined as an agent which functions to inhibit expression of a target gene, e.g., by RNAi. An siRNA can be chemically synthesized, it can be produced by in vitro transcription, or it can be produced within a host cell. siRNA molecules can also be generated by cleavage of double stranded RNA, where one strand is identical to the message to be inactivated.

In one embodiment, an siRNA agent is a double stranded RNA (dsRNA) molecule of about 15, 16, 17, 18, 19, 20, 21, 22, 23, 24, 25, 26, 27, 28, or 30 nucleotides in length, preferably about 15 to about 28 nucleotides, more preferably about 19, 20, 21, 22, 23, 24, or nucleotides in length, and more preferably about 19, 20, 21, 22, or 23 nucleotides in length, and can contain a 3′ and/or 5′ overhang on each strand having a length of about 1, 2, 3, 4, or 5 nucleotides. The length of the overhang is independent between the two strands, i.e., the length of the over hang on one strand is not dependent on the length of the overhang on the second strand. Preferably the siRNA is capable of promoting RNA interference through degradation or specific post-transcriptional gene silencing (PTGS) of the target messenger RNA (mRNA).

An siRNAs agent for use in the methods as disclosed herein also include small hairpin (also called stem loop) RNAs (shRNAs). In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow. These shRNAs can be contained in plasmids, retroviruses, and lentiviruses and expressed from, for example, the pol III U6 promoter, or another promoter (see, e.g., Stewart, et al. (2003) RNA April;9(4):493-501, incorporated by reference herein in its entirety).

The term “shRNA” as used herein refers to short hairpin RNA which functions as RNAi and/or siRNA species but differs in that shRNA species are double stranded hairpin-like structure for increased stability.

In some embodiments, the agent is an avimer. Avimers are multi-domain proteins with binding and inhibiting properties and are comprised typically of multiple independent binding domains linked together, and as such creates avidity and improved affinity and specificity as compared to conventional single epitope binding proteins such as antibodies. In some embodiments, one can use an avimer that is a protein or polypeptide that can bind simultaneously to a single protein target and/or multiple protein targets, as known as multi-point attachment in the art. Avimers are useful as therapeutic agents which function son multiple drug targets simultaneously for the progenitor cell and/or treatment of multifactorial diseases or disorders, for example multifactorial cancer malignanices or inflammatory disorders or autoimmune diseases.

In some embodiments, the agent is an antigomir. Antigomirs are oligonucleotides, for example synthetic oligonucleotides capable of gene silencing endogenous miRNAs.

The term “association” or “interaction” as used herein in reference to the association or interaction of an agent, e.g., siRNA, with a carrier particle, refers to any association between the agent, e.g., siRNA, with a carrier particle, e.g., a peptide carrier, either by a direct linkage or an indirect linkage. An indirect linkage includes an association between a agent, e.g., siRNA, and a carrier particle wherein said agent, e.g., siRNA, and said carrier particle are attached via a linker moiety, e.g., they are not directly linked. Linker moieties include, but are not limited to, e.g., nucleic acid linker molecules, e.g., biodegradable nucleic acid linker molecules. A nucleic acid linker molecule can be, for example, a dimer, trimer, tetramer, or longer nucleic acid molecule, for example an oligonucleotide of about 2, 3, 4, 5, 6, 7, 8, 9, 10, 11, 12, 13, 14, 15, 16, 17, 18, 19, 20, 21, 22, 23, 24, 25, or more nucleotides in length.

A direct linkage includes any linkage wherein a linker moiety is not required. In one embodiment, a direct linkage includes a chemical or a physical interaction wherein the two moieties, the therapeutic agent, e.g., siRNA, and the carrier particle, interact such that they are attracted to each other. Examples of direct interactions include non-covalent interactions, hydrophobic/hydrophilic, ionic (e.g., electrostatic, coulombic attraction, ion-dipole, charge-transfer), Van der Waals, or hydrogen bonding, and chemical bonding, including the formation of a covalent bond. Accordingly, in one embodiment, an agent, e.g., siRNA, and the carrier particle are not linked via a linker, e.g., they are directly linked. In a further embodiment, the therapeutic agent, e.g., siRNA, and the carrier particle are electrostatically associated with each other.

Agents delivered to leukocytes by the methods as disclosed herein include small molecules chemical and peptides to block intracellular signaling cascades, enzymes (kinases), proteasome function, lipid metabolism, cell cycle and membrane trafficking. Agents delivered by the methods of the present invention include agents that inhibit leukocyte extravasation or decrease vascular permeability. Such therapeutic agents can be useful in the treatment of, for example but not limited to, sustained inflammation, atherosclerosis, autoimmune diseases, ischemia-reperfusion injury and angiogenesis.

In another embodiment, an agent, for example a siRNA therapeutic agent as disclosed herein can be prepared to be delivered in a “prodrug” form. The term “prodrug” indicates a therapeutic agent that is prepared in an inactive form that is converted to an active form (i.e., drug) within the body or cells thereof by the action of endogenous enzymes or other chemicals and/or conditions.

In one embodiment, an agent is a protein, or growth factor, cytokine, immunomodulating agent, or other protein, including proteins which when expressed present an antigen which stimulates or suppresses the immune system.

In another embodiment, the agent is a diagnostic agent capable of detection in vivo following administration by a leukocyte delivery agent. Exemplary diagnostic agents include electron dense material, magnetic resonance imaging agents, radiopharmaceuticals and fluorescent molecules. Radionucleotides useful for imaging include radioisotopes of copper, gallium, indium, rhenium, and technetium, including isotopes ⁶⁴Cu, ⁶⁷Cu, ¹¹¹In, ^{99m}Tc, ⁶⁷Ga or ⁶⁸Ga. Imaging agents disclosed by Low et al. in U.S. Pat. No. 5,688,488, incorporated herein by reference, are useful in the liposomal complexes described herein.

In one aspect of the method, the liposome product is detectably labeled with a label selected from the group including a radioactive label, a fluorescent label, a non-fluorescent label, a dye, or a compound which enhances magnetic resonance imaging (MRI). In one embodiment, the liposome product is detected by acoustic reflectivity. The label may be attached to the exterior of the liposome or may be encapsulated in the interior of the liposome.

In some embodiments, an agent can be an imaging agent. In order to function as a suitable agent for medical imaging, the effector agent is useful in a molecular imaging diagnosis procedure, for example but not limited to, magnetic resonance (MR) imaging. Delivery of such imaging agents using the methods and compositions as disclosed herein can be used to image extent of leukocyte extravasation and/or vascular permeability by MRI or PET for example. Contrast enhancement Can be provided by gadolinium, for example, gadolinium in the form of Gd-DTPA-aminohexanoic acid. Other imaging agents are useful in the methods as disclosed herein include, for example other lanthanide ion coordination complexes can allow for even greater enhanced relaxation at higher field strength (Aime, S., et al., Chem. Soc. Rev. 27:19-29, 1998; Aime et al., J. Mannet. Reson. Iman. 16:394-406, 2002). Paramagnetic CES T agents are useful as imaging agents in the methods and compositions as disclosed herein, for example as Eu+3, Tb+3, Dy+3, Er+3, Tm+3, or Yb+3 alter tissue contrast via chemical exchange saturation transfer of presaturated spins to bulk I water (Elst, L. V., et al., Mann. Reson. Med. 47:1121-1130, 2002). In some embodiments, more than one imaging agent can be used simultaneously in the composition and methods of the present invention, with techniques available for attachment of multiple imaging agents, for example Gd-DTPA to proteins to enhance the MR signal known by persons of ordinary skill in the art. The T1 acceleration and contrast enhancement of Gd and especially Fe have been shown to saturate at very high field strength, however, while these other lanthanides do not, thus taking full advantage of the increased resolution of very high field strengths.

In some embodiments, an imaging agent is useful as diagnostic agent capable of detection in vivo following administration. Exemplary imaging agents useful for diagnostic purposes include electron dense material, magnetic resonance imaging agents, radiopharmaceuticals and fluorescent molecules. Radionucleotides useful for imaging include radioisotopes of copper, gallium, indium, rhenium, and technetium, including isotopes ⁶⁴Cu, ⁶⁷Cu, ¹¹¹In, ^{99m}Tc, ⁶⁷Ga or ⁶⁸Ga. Imaging agents disclosed by Low et al. in U.S. Pat. No. 5,688,488, incorporated herein by reference, are also useful in the compositions as disclosed herein.

### Insoluble Agents

One aspect of the invention relates to compositions and methods to simultaneously deliver at least one insoluble agent and at least one soluble agent to a target cell, such as or example a lymphocyte or an endothelial cell. An insoluble agent is also referred to as a water-insoluble agent, a hydrophobic agent or a lipophilic agent, as those terms are defined herein.

Any insoluble agent is contemplated for delivery to a target cell using the methods and compositions as disclosed herein. Examples of insoluble agents include, for example, but are not limited to paxlitaxel, also referred to as TAXOL® (Bristol-Myers Squibb), ONXAL™, ABRAXANE™ (Abraxis Oncology). Camptothecin (CPT) and its derivatives are considered to be among the most effective anticancer drugs of the 21st century. Although studies have demonstrated their effectiveness against carcinomas of the stomach, colon, neck and bladder, as well as against breast cancer, small-cell lung cancer and leukemia in vitro, clinical application of CPT in humans has only been carried out with CPT derivatives that have improved water solubility. Accordingly, CPT is an example of an insoluble agent for delivery to target cells using the compositions and methods as disclosed herein.

In some embodiments, an insoluble agent useful in the methods as disclosed herein is a therapeutic agent for the treatment of tumors which can be delivered to leukocytes by the methods as described herein, for example such therapeutic agents are chemotherapy agents. The term “chemotherapeutic agent” or “chemotherapy agent” are used interchangeably herein and refers to an agent that can be used in the treatment of cancers and neoplasms that are capable of treating such a disorder. In some embodiments, a chemotherapeutic agent can be in the form of a prodrug which can be activated to a cytotoxic form. Chemotherapeutic agents are commonly known by persons of ordinary skill in the art and are encompassed for use in the present invention. For example, chemotherapeutic drugs for the treatment of tumors include, but are not limited to: temozolomide (TEMODAR®), procarbazine (MATULANE®), and lomustine (CCNU, Cyclin U). Chemotherapy given intravenously (by IV, via needle inserted into a vein) includes vincristine (ONCOYIN® or VINCASAR PFS®), cisplatin (PLATINOL®), carmustine (BCNU, BiCNU), and carboplatin (PARAPLATIN®), Mexotrexate (RHEUMATREX® or TREXALL®).

Accordingly in some embodiments, the methods to use the leukocyte delivery agents as disclosed herein can also be used for diagnostic purposes, for example but not limited to visualization of vascular permeability and/or leukocyte extravasation in a subject, for example visualization of vascular permeability and/or leukocyte extravasation of subject with atherosclerosis, cancer, an autoimmune disease or following ischemia-reperfusion injury. In further embodiments, the compositions and methods of the present invention are useful for monitoring the effect of a therapeutic intervention and/or for prognostic purposes. For example, in some embodiments the present invention can be used for monitoring the efficacy of a therapeutic treatment in a subject treated with a therapy for atherosclerosis, cancer, an autoimmune disease, ischemia-reperfusion and monitoring the reduction of vascular permeability and/or leukocyte extravasation in the subject.

Accordingly, as disclosed herein the method provides a means to deliver nucleic acids, such as siRNA, nucleic, acids, nucleic acid analogues, miRNA, miRNA mimetics, antigomirs and the like to leukocytes in vivo and in vivo. The methods as disclosed herein are useful for delivering agents to leukocytes cells in vitro, in vivo or ex vivo for multiple purposes, such as (i) research purposes including but not limited to investigating or studying leukocytes function and responses, increasing our understanding of leukocytes-endothelial interaction, leukocyte extravasasation, and response to agents as well as general assays for reducing vascular permeability and vascular permeability inhibitor assays, and (ii) therapeutic purposes.

Other examples of some insoluble agents for use in the compositions and methods as disclosed herein include, but are not limited to, immunosuppressive and immunoactive agents, anti-angiogenic agents, antiviral and antifungal agents, antineoplastic agents, analgesic and anti-inflammatory agents, antibiotics, anti-epileptics, anesthetics, hypnotics, sedatives, antipsychotic agents, neuroleptic agents, antidepressants, anxiolytics, anticonvulsant agents, antagonists, neuron blocking agents, anticholinergic and cholinomimetic agents, antimuscarinic and muscarinic agents, antiadrenergic and antarrhythmics, antihypertensive agents, antineoplastic agents, hormones, and nutrients. A detailed description of these and other suitable drugs may be found in Remington's Pharmaceutical Sciences, 18th edition, 1990, Mack Publishing Co. Philadelphia, Pa. which is hereby incorporated by reference.

Insoluble agents or insoluble drugs can have pharmaceutical efficacy in a number of therapeutic and diagnostic imaging areas. Non-limiting classes of compounds and agents from which poorly water soluble drugs that melt without decomposition and are useful in this invention can be selected include anesthetic agents, ace inhibiting agents, antithrombotic agents, anti-allergic agents, anti-angiogenic agents, antibacterial agents, antibiotic agents, anticoagulant agents, anticancer agents, antidiabetic agents, antihypertension agents, antifungal agents, antihypotensive agents, antiinflammatory agents, antimicotic agents, antimigraine agents, antiparkinson agents, antirheumatic agents, antithrombins, antiviral agents, beta blocking agents, bronchospamolytic agents, calcium antagonists, cardiovascular agents, cardiac glycosidic agents, carotenoids, cephalosporins, contraceptive agents, cytostatic agents, diuretic agents, enkephalins, fibrinolytic agents, growth hormones, immunosupressants, insulins, interferons, lactation inhibiting agents, lipid-lowering agents, lymphokines, neurologic agents, prostacyclins, prostaglandins, psycho-pharmaceutical agents, protease inhibitors, magnetic resonance diagnostic imaging agents, reproductive control hormones, sedative agents, sex hormones, somatostatins, steroid hormonal agents, vaccines, vasodilating agents, and vitamins.

Additional examples of insoluble agents for use in the compositions and methods as disclosed herein include agents which melt without decomposition in admixtures, suspensions, dispersions, and homogenates of this invention, preferably in a temperature range from about physiological temperature 37° C. to about 275° C., and more preferably in a temperature range from just above physiological temperature, about 40° C., to about 230° C. Non-limiting examples of representative suitable insoluble agents which can be delivered by the methods and compositions as disclosed herein can be selected from the group consisting albendazole (m.p. 208-21° C.), albendazole sulfoxide, alfaxalone (m.p. 172-174° C.), acetyldigoxin, acyclovir analogs melting at or below 275° C., alprostadil, aminofostin, anipamil, antithrombin III, atenolol (m.p. 146-148° C.), azidothymidine, beclobrate (m.p. 200-204° C.), beclomethasone (m.p. 117-120° C.), belomycin, beuzocaine (m.p. 88-90° C.) and derivatives, beta carotene (m.p. 183° C.), beta endorphin, beta interferon, bezafibrate (m.p. 186° C.), binovum, biperiden (m.p. 112-116° C.), bromazepam (m.p. 237-238° C.), bromocriptine, bucindolol, buflomedil (m.p. 192-193° C.), bupivacaine (m.p. 107-108° C.), busulfan (m.p. 114-118° C.), cadralazine (m.p. 160-162° C.), campotothecin (m.p. 264-267 and 275° C.), canthaxanthin (m.p. 217° C.), captopril (m.p. 103-104° C.), carbamazepine (m.p. 190-193° C.), carboprost, cefalexin, cefalotin, cefamandole (m.p. 190° C.), cefazedone, cefluoroxime, cefirienoxime, cefoperazone (m.p. 169-171° C.), cefotaxime, cefoxitin (m.p. 149-150° C.), cefsulodin (m.p. 175° C.), ceftizoxime, chiorambucil (m.p. 64-66° C.), chromoglycinic acid, ciclonicate (m.p. 127-128° C.), ciglitazone, clonidine (m.p. 130° C.), cortexolone, corticosterone (m.p. 180-182° C.), cortisol (m.p. 212-220° C.), cortisone (m.p. 220-224° C.), cyclophosphamide (m.p. 41-45° C.), cyclosporin A (m.p. 148-151° C.) and other cyclosporins, cytarabine (m.p. 212-213° C.), desocryptin, desogestrel (m.p. 109-110° C., dexamethasone esters such as the acetate (m.p. 238-240° C.), dezocine, diazepam (m.p. 125-126° C.), diclofenac, dideoxyadenosine (m.p. 160-163° C.), dideoxyinosine, digitoxin (m.p. 256-257° C.), digoxin, dihydroergotamine (m.p. 239° C.), dihydroergotoxin, diltiazem (m.p. 207-212° C.), dopamine antagonists, doxorubicin (m.p. 229-231° C.), econazole (m.p. 87° C.), endralazine (m.p. 185-188° C.), enkephalin, enalapril (m.p. 143-145° C.), epoprostenol, estradiol (m.p. 173-179° C.), estramustine (m.p. 104-105° C.), etofibrate (m.p. 100° C.), etoposide (m.p. 236-251° C.), factor ix, factor viii, felbamate (m.p. 151-152° C.), fenbendazole (m.p. 233° C.), fenofibrate (m.p. 79-82° C.), flunarizin (m.p. 252° C.), flurbiprofen (m.p. 110-111° C.), 5-fluorouracil (m.p. 282-283° C.), flurazepam (m.p. 77-82° C.), fosfomycin (m.p. 94° C.), fosmidomycin, furosemide (m.p. 206° C.), gallopamil, gamma interferon, gentamicin (m.p. 102-108° C.), gepefrine (m.p. 155-158° C.), gliclazide (m.p. 180-182° C.), glipizide (m.p. 208-209° C.), griseofulvin (m.p. 220° C.), haptoglobulin, hepatitis B vaccine, hydralazine (m.p. 172-173° C.), hydrochlorothiazide (m.p. 273-275° C.), hydrocortisone (m.p. 212-220° C.), ibuprofen (m.p. 75-77° C.), ibuproxam (m.p. 119-121° C.), indinavir, indomethacin (m.p. 155° C.), iodinated aromatic x-ray contrast agents melting below 275° C. such as iodamide (m.p. 255-257° C.), ipratropium bromide (m.p. 230-232° C.), ketoconazole (m.p. 146° C.), ketoprofen (m.p. 94° C.) ketotifen (m.p. 152-153° C.), ketotifen fumarate (m.p. 192° C.), K-Strophanthin (m.p. 175° C.), labetalol, lactobacillus vaccine, lidocaine (m.p. 68-69° C.), lidoflazine (m.p. 159-161° C.), lisuride (m.p. 1.86° C.), lisuride hydrogen maleate (m.p. 200° C.), lorazepam (m.p. 166-168° C.), lovastatin, mefenamic acid (m.p. 230-231° C.), meiphalan (m.p. 182-183° C.), memantine, mesulergin, metergoline (m.p. 146-149° C.), methotrexate (m.p. 185-204° C.), methyldigoxin (m.p. 227-231° C.), methylprednisolone (m.p. 228-237° C.), metronidazole (m.p. 158-160° C.), metisoprenol, metipranolol (m.p. 105-107° C.), metkephamide, metolazone (m.p. 253-259° C.), metoprolol, metoprolol tartrate, miconazole (m.p. 135° C.), miconazole nitrate (m.p. 170 and 185° C.), minoxidil (m.p. 248° C.), misonidazol, molsidomine, nadolol (m.p. 124-136° C.), nafiverine (m.p. 220-221° C.), nafazatrom, naproxen (m.p. 155° C.), natural insulins, nesapidil, nicardipine (m.p. 168-170° C.), nicorandil (m.p. 92-93° C.), nifedipine (m.p. 172-174° C.), niludipin, nimodipine, nitrazepam (m.p. 224-226° C.), nitrendipine, nitrocamptothecin, 9-nitrocamptothecin, oxazepam (m.p. 205-206° C.), oxprenolol (m.p. 78-80° C.), oxytetracycline (m.p. 181-182° C.), penicillins such as penicillin G benethamine (m.p. 147-147° C.), penecillin 0 (m.p. 79-81° C.), phenylbutazone (m.p. 105° C.), picotamide, pindolol (m.p. 171-173° C.), piposulfan (m.p. 175-177° C.), piretanide (m.p. 225-227° C.), piribedil (m.p. 98° C.), piroxicam (m.p. 198-200° C.), pirprofen (m.p. 98-100° C.), plasminogenic activator, prednisolone (m.p. 240-241° C.), prednisone (m.p. 233-235° C.), pregneninolone (m.p. 193° C.), procarbazine, procaterol, progesterone (m.p. 121° C.), proinsulin, propafenone, propentofylline, propofol, propranolol (m.p. 96° C.), rifapentine, simvastatin, semi-synthetic insulins, sobrerol (m.p. 130° C.), somatostatin and its derivatives, somatotropin, stilamin, sulfinalol whose hydrochloride melts at 175° C., sulfinpyrazone (m.p. 136-137° C.), suloctidil (m.p. 62-63° C.), suprofen (m.p. 124° C.), suiprostone, synthetic insulins, talinolol (m.p. 142-144° C.), taxol, taxotere, testosterone (m.p. 155° C.), testosterone propionate (m.p. 118-122° C.), testosterone undecanoate, tetracane HI (m.p. .about. 450° C.), tiaramide (HCl m.p. 159-161° C.), tolmetin (m.p. 155-157° C.), tranilast (m.p. 211-213° C.), triquilar, tromantadine (HCl m.p. 157-158° C.), urokinase, valium (m.p. 125-126° C.), verapamil (m.p. 243-246° C.), vidarabine, vidarabine phosphate sodium salt, vinblastine (m.p. 211-216° C.), vinburin, vincamine (m.p. 232-233° C.), vincristine (m.p. 218-220° C.), vindesine (m.p. 230-232° C.), vinpocetine (m.p. 147-153° C.), vitamin A (m.p. 62-64° C.), vitamin E succinate (m.p. 76-78° C.), and x-ray contrast agents. Agents can be neutral species or basic or acidic as well as salts such as exist in the presence of an aqueous buffer.

In some embodiments, an insoluble agent for delivery using the composition and methods as disclosed herein can be an insoluble nucleic acid, an insoluble nucleic acid construct, or insoluble protein or peptide. Insoluble nucleic acid constructs may comprise for example, but without limitation bare nucleic acid molecules, RNAi, small nucleic acid particles, viral vectors, associated viral particle vectors, nucleic acids present in a vesicle, or the like.

### Encapsulating or Entrapping Agents in Carrier Particles

In another embodiment where the agent is a hydrophilic agent, for example a nucleic acid agent such as DNA, RNA, siRNA, plasmid DNA, short-hairpin RNA, small temporal RNA (stRNA), microRNA (miRNA), RNA mimetics, or heterochromatic siRNA, or where the agent is a nucleic acid agent that has a charged backbone that prevents efficient encapsulation in the lipid particle, such agents can be condensed with a cationic polymer, e.g., PEI, polyamine spermidine, and spermine, or cationic peptide, e.g., protamine and polylysine, prior to encapsulation in the lipid particle. In some embodiments, the agent is not condensed with a cationic polymer.

In some embodiments, an agent is encapsulated in the lipid particle or other polymeric nanoparticle in the following manner: The lipid particle or polymeric nanoparticle, in which can additionally comprise a cryoprotectant and/or a targeting moiety is provided lyophilized. The agent is in an aqueous solution. The agent in aqueous solution is utilized to rehydrate the lyophilized lipid particle or nanoparticle. Thus, the agent is encapsulated in the rehydrated lipid particle or polymeric nanoparticle. An example of encapsulation of a soluble agent within the lipid particle includes, but not limited to, soluble agents such as nucleic acids as demonstrated in the Examples for the encapsulation of the cDNAs for Ku70 or CD1.

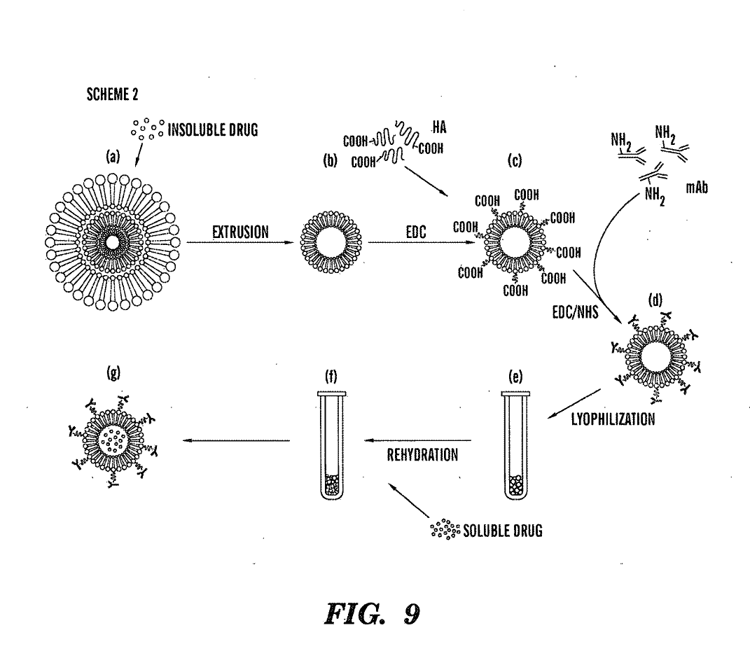

In another embodiment, two or more agents can be delivered by carrier particle, for example a lipid particle or polymeric nanoparticles by the methods as disclosed herein and in Example 7. In such embodiments, one agent can be an insoluble (i.e. hydrophobic or lipohilic) agent and the other agent a soluble (i.e. hydrophilic) agent. An insoluble (or hydrophobic/lipophilic) agent can be added to the lipid particle during formation of the lipid particle and can associate with the lipid portion of the lipid particle, as demonstrated in FIG. 9. The soluble agent (i.e. hydrophilic agent) is associated with the lipid particle by being added in the aqueous solution during the rehydration of the lyophilized lipid particle, also demonstrated in FIG. 9. An exemplary embodiment of two agent delivery is described in Example 7 herein, where the soluble agent is a siRNA, which is encapsulated or entrapped in the aqueous interior of aliposome, and where Taxol which is an insoluble (hydrophobic) agent and poorly soluble in aqueous solution is associated with the lipid portion of the liposome carrier particle. As used herein, “poorly soluble in aqueous solution” refers to a composition that is less that 10% soluble in water.

Any suitable lipid: pharmaceutical agent ratio that is efficacious is contemplated by the present invention. In some embodiments, the lipid: pharmaceutical agent molar ratios include about 2:1 to about 30:1, about 5:1 to about 100:1, about 10:1 to about 40:1, about 15:1 to about 25:1.

In some embodiments, the loading efficiency of therapeutic or pharmaceutical agent is a percent encapsulated pharmaceutical agent of about 50%, about 60%, about 70% or greater. In one embodiment, the loading efficiency for a soluble agent is a range from 50-100%. In some embodiments, the loading efficiency of an insoluble agent to be associated with the lipid portion of the lipid particle, (i.e. a pharmaceutical agent poorly soluble in aqueous solution), is a percent loaded pharmaceutical agent of about 50%, about 60%, about 70%, about 80%, about 90%, about 100%. In one embodiment, the loading efficiency for a hydrophobic agent in the lipid layer is a range from 80-100%.

In one aspect of the method, a leukocyte targeting agent can be detectably labeled, for example it can comprise a carrier particle such as a liposome or polymeric nanoparticle is detectably labeled with a label selected from the group including a radioactive label, a fluorescent label, a non-fluorescent label, a dye, or a compound which enhances magnetic resonance imaging (MRI). In one embodiment, the liposome product is detected by acoustic reflectivity. The label may be attached to the exterior of the liposome or may be encapsulated in the interior of the liposome.

In one embodiment, the invention is directed to a method to encapsulate nucleic acids, e.g., plasmid DNA, DNA fragments, short interfering RNA (siRNA), short-hairpin RNA, small temporal RNA (stRNA), microRNA (miRNA), RNA mimetics, or heterochromatic siRNA. In one embodiment, nucleic acids are condensed with a cationic polymer, e.g., PEI, polyamine spermidine, and spermine, or a cationic peptide, e.g., protamine and polylysine, and encapsulated in the lipid particle.

### Uses of the Compositions

Another aspect of the present invention relates to use of the compositions as disclosed herein comprising a carrier particle (comprising both an insoluble agent and a soluble agent) associated with a targeting moiety to deliver the insoluble agent and soluble agent to selected a target cell. In some embodiments, the insoluble agent and soluble agent have synergistic or additive effects. As an illustrative example only, a leukocyte delivery agent or endothelial cell delivery agent can be used to deliver two agents which function by two independent mechanisms or cellular pathways for a common outcome. For example and as disclosed herein and in the Examples, the inventors demonstrate the use of a leukocyte delivery agent to deliver a soluble anti-cancer agent and an insoluble anti-cancer agent to kill an immortalized cancer cell line using separate biological cell death pathways. In Example 7, the inventors demonstrate use of a leukocyte delivery agent to deliver an insoluble agent (i) a siRNA to decrease the expression of CyDI which functions to inhibit the continuation of the cell cycle, and (ii) TAXOL® which inhibits cell cycle progression by interfering with the mechanisms which are necessary for dividing cells. Thus, Example 7 demonstrates the delivery of two agents; a soluble agent and an insoluble agent which function by different mechanisms to inhibit cell cycle progression.

By way of another example, one can use the compositions as disclosed herein for antivirus small molecule therapy in the treatment of a subject with a disease caused by a virus. For example, one deliver a soluble agent such as an anti-HIV siRNA, such as for example HIV tat/rev RNAis, in combination with an insoluble anti-HIV agent, such as for example the reverse-transcriptase inhibitor AZT/azidothymidine, where the effect of both the insoluble agent (the anti-HIV RNAi) and soluble agent (AZT) are additive to one another as they function by different mechanisms and different pathways to inhibit HIV viral replication, thus are additive to each other with respect to they both function to inhibit HIV viral replication by independent biological pathways. Examples of other combinations of anti-viral RNAi and anti-HIV therapies is discussed in Li et al, Annals of the New York Academy of Sciences, 1082; 172-179 and Li, et al, 2005; Mol. Ther. 12: 900-909 which are incorporated herein in their entirety by reference.

In another example, one can use the compositions as disclosed herein wherein a carrier particle is used to deliver both an insoluble agent and a soluble agent to a target cell for dual delivery of a first therapeutic agent and a second agent which attenuates or decreases any side-effects caused by the first therapeutic agent. Stated another way, the composition of the present invention can be used to deliver at least two agents, where one agent mitigates any adverse side effects caused by the other agent. As an exemplary example, one can use the compositions as disclosed herein to deliver therapeutic agent which is a soluble agent, such as a RNAi and at the same time deliver an insoluble agent, such as an immune suppressant, such as Cyclosporin (or FK-506 also known as Tarcrolimus (PROGRAF®) or rapamycin also known as sirolimus (RAPAMUNE®)) which decrease or mitigate the adverse side effects caused by the soluble RNAi agent.

Similarly and in the converse situation, in some embodiments one can use the compositions as disclosed herein to deliver therapeutic agent which is an insoluble agent, such as cisplatin (also known as PLATINOL®, PLATINOL®-AQ or CDDP) or other platinum based chemotherapy drugs at the same time deliver soluble agent, such an RNAi or other soluble agent, such as TAVOCEPT™ (also known as BNP7787) or procaine hydrochloride (P.HCl) which function to improve the therapeutic index of cisplatin by reduction of its nephro- and hemotoxicity and also increases its antitumor activity, thus the dual delivery of such agents is beneficial to increase therapeutic efficacy of one agent yet decreasing or mitigating the adverse side effects caused by the cisplatin agent.

In another embodiment, one can use the compositions as disclosed herein to deliver therapeutic agent which is an soluble agent, such as antibody or peptide or polypeptide agent and at the same time deliver an insoluble agent, such an nucleic acid which is in the insoluble format, for example an insoluble RNAi or a lipophilic RNAi which prevents the immune response or formation of antibodies directed to the antibody, peptide or polypeptide soluble therapeutic agent.

In some embodiments one can use the compositions as disclosed herein to deliver antimicrobial therapeutic agents, where one agent functions as an antimicrobial agent and the second agent functions to inhibit resistance genes to the antimicrobial agents. By way of a non-limiting example, in some embodiments, an insoluble therapeutic agent, such as an insoluble antimicrobial agent such as for example but not limited to Colimycin or polymycin E, or other antimicrobial lipopeptides and cyclic lipopeptides can be delivered at the same time as soluble agent, such an soluble RNAi, which functions to inhibit at least one resistance gene to the antimicrobial peptide. Alternatively one can deliver an soluble antimicrobial agent and an insoluble inhibitor of a resistance gene, such as for example, where one of the insoluble or soluble inhibitors to a resistance gene is selected from the group comprising; mefloquine, venturicidin A, diaryquinoline, betaine aldehyde chloride, acivcin, psicofuraine, buthionine sulfoximine, diaminopemelic acid, 4-phospho-D-erythronhydroxamic acid, motexafin gadolinium and/or xycitrin or modified versions or analogues thereof.

Any combination of a soluble agent and an insoluble agent is contemplated by the present invention. Some examples of drug combination which can be delivered to a target cell by the compositions and methods as disclosed herein include, for example, anastrozole (ARIMIDEX®, AstraZeneca) in combination with trastuzumab (HERCEPTIN®, Roche),

Accordingly, as demonstrated herein, the compositions as disclosed herein can be used to for dual delivery of agents which function by two independent mechanisms for the same biological outcome. Stated another way, the compositions as disclosed herein can be used to for dual delivery of at least one insoluble agent and at least one soluble agent which have additive effects by two independent mechanisms for the same biological outcome.

In another embodiment, the compositions as disclosed herein can be used to for dual delivery of agents which function synergistically together. Another example of dual delivery of agents which function synergistically is a small molecule and a nucleic acid. For example, Enoxacin (also known as Penetrex) and its derivatives enhance siRNA-mediated mRNA degradation. Thus, in one embodiment the compositions and methods as disclosed herein can be used for dual delivery of a siRNA (Shan et al. (2008) Nature Biotech. 26, 933-940) plus enoxacin for synergistic biological effects. This is synergistic because Enoxacin has essentially no effect in mRNA degradation by itself, but enhances siRNA-mediated degradation. In some embodiments, the delivery of at least one siRNA and another compound from the quinolone family of synthetic antibacterial compounds, such as for example ciprofloxacin, ofloxacin, norfloxacin, and difloxacin have shown similar effects as Enoxacin in enhancing siRNA-mediated degradation, and accordingly can be simultaneously delivered with a siRNA using the dual-delivery system as disclosed herein.

### Kits

Encompassed in the invention is a leukocyte-selective delivery kit, comprising ready-to-used lyophilized leukocyte-selective delivery agent as disclosed herein, where in the leukocyte-selective delivery agent comprises targeting moieties for targeting activated leukocyte cells which are associated with carrier particles, where the leukocyte-selective delivery agent is ready for drug or agent encapsulation.

Encompassed in the invention is a leukocyte-selective delivery kit comprising ready-to-used lyophilized leukocyte-selective delivery agent. For example, the targeting moieties associated to the carrier particle of the leukocyte-selective delivery agent lipid particle of the kit may be antibodies against integrins present on leukocytes, such as antibodies selective for integrins or activated integrins such as but not limited to; LFA-1 (αLβ2), Mac-1 (αMβ2), p150.95 (αXβ2), αDβ2, VLA-4 (α4β1), and 137 (α4β7 and αEβ7) or fragments, homologues or variants thereof for targeting the leukocyte-selective delivery agent to leukocytes and activated leukocytes. Alternatively, a targeting moiety of a leukocyte-selective delivery kit may be integrin ligands receptors or fragments or variants thereof, such as those present on epithelial cells for example, ICAM-1, ICAM-2, ICAM-3, VCAM-1, MAdCAM-1, E-cadherin, JAM-1, JAM-2 and JAM-3 or variants or homologues or fragments thereof.

The lyophilized carrier particle of the leukocyte-selective delivery kit can be rehydrated directly in the drug or agent solution for drug or agent encapsulation respectively. The targeting moiety may be functional fragments of an antibody.

### Therapeutic Administration

One aspect of the present invention provides a composition which comprises a leukocyte delivery agent as disclosed herein, where the leukocyte delivery agent comprises a targeting moiety, for example an anti-integrin antibody or fragment thereof or an integrin ligand or fragment or variant thereof, and a carrier particle, wherein an agent is associated with the carrier particle. In some embodiments, the carrier particle is a liposomal or polymeric nanoparticles such as a liposome.

In some embodiments, the composition comprises a targeting moiety, for example an anti-integrin antibody or integrin ligand or fragment thereof, conjugated to a carrier particle, and at least one agent. In some embodiments, where the composition comprises a plurality of targeting moieties and carrier particles, there can be various different of targeting moieties, which can be conjugated all to the same type of carrier particle, or different carrier particles. By way of a non-limiting example, the composition can comprise an anti-integrin antibody as a first targeting moiety which is conjugated to a carrier particle such as a liposome or polymeric nanoparticle, and the composition can also comprise another targeting moiety-carrier particle conjugate comprising a integrin ligand or fragment thereof as the targeting moiety and a different type of carrier particle. In other words, the composition can comprise a plurality of targeting moiety-carrier particle conjugates with agents associated with the carrier particles. Accordingly, in some embodiments the targeting moiety and carrier particle of each targeting moiety-carrier particle conjugate can be the same or different types of targeting moiety and carrier particles respectively. In some embodiments, the composition comprises a plurality of targeting moiety conjugated to a plurality of different types of carrier particles. In some embodiments, any combination of targeting moiety can be used with any combination of carrier particle. Accordingly, depending on the carrier particles present in the composition will also determines the types of agents also in the composition. As a non-limiting example, some targeting moiety-carrier particle conjugates may comprise a hydrophobic agent, such as a small molecule, and some may comprise hydrophilic agents such as a nucleic acid agent or RNAi agent, and some may contain both a hydrophobic agent and a hydrophilic agent.

In further embodiments, a composition of the present invention can comprise a plurality of leukocyte delivery agents, for e.g. a plurality of targeting moieties associated with carrier particles, the agents also present in the composition that are associated with the carrier particle can also be different. For instance, an agent associated with the carrier particle can be a different type of effector agent, for example nucleic acid agent or a peptide agent. In some embodiments, an agent can be different variant of the same type of agent, for example if the agent is a nucleic acid, the composition can comprise both RNA and DNA agents. In further embodiments, the composition can comprise a plurality of agents that are variants of the same type of agent, for example variants or derivatives of siRNA. By way of a non-limiting example, the composition can comprise a plurality of RNAi agents that associate with the carrier peptide, where the RNAi agents are different, for example the RNAi agent silences different gene targets or targets different regions on the same gene.

Compositions as disclosed herein comprising leukocyte delivery agents can be administered by any convenient route, including parenteral, enteral, mucosal, topical, e.g., subcutaneous, intravenous, topical, intramuscular, intraperitoneal, transdermal, rectal, vaginal, intranasal or intraocular. In one embodiment, the compositions as disclosed herein are not topically administered. In one embodiment, the delivery is by oral administration of the composition formulation. In one embodiment, the delivery is by intranasal administration of the composition, especially for use in therapy of the brain and related organs (e.g., meninges and spinal cord). Along these lines, intraocular administration is also possible. In another embodiment, the delivery means is by intravenous (i.v.) administration of the composition, which is especially advantageous when a longer-lasting i.v. formulation is desired. Suitable formulations can be found in Remington's Pharmaceutical Sciences, 16th and 18th Eds., Mack Publishing, Easton, Pa. (1980 and 1990), and Introduction to Pharmaceutical Dosage Forms, 4th Edition, Lea & Febiger, Philadelphia (1985), each of which is incorporated herein by reference.

Compositions comprising leukocyte delivery agents can be administered in prophylatically or therapeutically effective amounts. The targeted delivery compositions as disclosed herein can be administered along with a pharmaceutically acceptable carrier. A prophylatically or therapeutically effective amount means that amount necessary, at least partly, to attain the desired effect, or to delay the onset of, inhibit the progression of, or halt altogether, the onset or progression of the particular disease or disorder being treated. Such amounts will depend, of course, on the particular condition being treated, the severity of the condition and individual patient parameters including age, physical condition, size, weight and concurrent treatment. These factors are well known to those of ordinary skill in the art and can be addressed with no more than routine experimentation. It is preferred generally that a maximum dose be used, that is, the highest safe dose according to sound medical judgment. It will be understood by those of ordinary skill in the art, however, that a lower dose or tolerable dose can be administered for medical reasons, psychological reasons or for virtually any other reasons.

In the preparation of pharmaceutical formulations comprising leukocyte delivery agent as disclosed herein in the form of dosage units for oral administration the compound selected can be mixed with solid, powdered ingredients, such as lactose, saccharose, sorbitol, mannitol, starch, amylopectin, cellulose derivatives, gelatin, or another suitable ingredient, as well as with disintegrating agents and lubricating agents such as magnesium stearate, calcium stearate, sodium stearyl fumarate and polyethylene glycol waxes. The mixture is then processed into granules or pressed into tablets.

Soft gelatin capsules can be prepared with capsules containing a mixture of the active compound or compounds of the invention in vegetable oil, fat, or other suitable vehicle for soft gelatin capsules. Hard gelatin capsules can contain granules of the active compound. Hard gelatin capsules can also contain the targeted delivery composition including the targeting moiety and the carrier particle as well as the therapeutic agent in combination with solid powdered ingredients such as lactose, saccharose, sorbitol, mannitol, potato starch, corn starch, arnylopectin, cellulose derivatives or gelatin.

Dosage units for rectal or vaginal administration can be prepared (i) in the form of suppositories which contain the active substance mixed with a neutral fat base; (ii) in the form of a gelatin rectal capsule which contains the active substance in a mixture with a vegetable oil, paraffin oil or other suitable vehicle for gelatin rectal capsules; (iii) in the form of a ready-made micro enema; or (iv) in the form of a dry micro enema formulation to be reconstituted in a suitable solvent just prior to administration.

Liquid preparations for oral administration can be prepared in the form of syrups or suspensions, e.g. solutions or suspensions containing from 0.2% to 20% by weight of the active ingredient and the remainder consisting of sugar or sugar alcohols and a mixture of ethanol, water, glycerol, propylene glycol and polyethylene glycol. If desired, such liquid preparations can contain coloring agents, flavoring agents, saccharin and carboxymethyl cellulose or other thickening agents. Liquid preparations for oral administration can also be prepared in the form of a dry powder to be reconstituted with a suitable solvent prior to use.

Solutions for parenteral administration can be prepared as a solution of a compound of the invention in a pharmaceutically acceptable solvent, preferably in a concentration from 0.1% to 10% by weight. These solutions can also contain stabilizing ingredients and/or buffering ingredients and are dispensed into unit doses in the form of ampoules or vials. Solutions for parenteral administration can also be prepared as a dry preparation to be reconstituted with a suitable solvent extemporaneously before use.

The methods to deliver the leukocyte delivery agent as disclosed herein can also be delivered orally in granular form including sprayed dried particles, or complexed to form micro or nanoparticles.

Furthermore, local administration of the leukocyte delivery agent as disclosed herein to treat malignancy or cancers by interstitial chemotherapy using surgically implanted, biodegradable implants is known. For example, a polyanhydride polymer, Gliadel® (Stolle R & D, Inc., Cincinnati, Ohio) a copolymer of poly-carboxyphenoxypropane and sebacic acid in a ratio of 20:80 has been used to make implants, intracranially implanted to treat malignant gliomas. Polymer and BCNU can be co-dissolved in methylene chloride and spray-dried into microspheres. The microspheres can then be pressed into discs 1.4 cm in diameter and 1.0 mm thick by compression molding, packaged in aluminum foil pouches under nitrogen atmosphere and sterilized by 2.2 megaRads of gamma irradiation. The polymer permits release of carmustine over a 2-3 week period, although it can take more than a year for the polymer to be largely degraded. Brem, H., et al, Placebo-Controlled Trial of Safety and Efficacy of Intraoperative Controlled Delivery by Biodegradable Polymers of Chemotherapy for Recurrent Gilomas, Lancet 345; 10081012:1995.

In addition to polymeric implants, osmotic pumps can also be utilized for delivery of the leukocyte delivery agent composition of the present invention by continuous infusion. An osmotic minipump contains a high-osmolality chamber that surrounds a flexible, yet impermeable, reservoir filled with the targeted delivery composition-containing vehicle. Subsequent to the subcutaneous implantation of this minipump, extracellular fluid enters through an outer semi-permeable membrane into the high-osmolality chamber, thereby compressing the reservoir to release leukocyte delivery agent at a controlled, pre-determined rate. The leukocyte delivery agent composition, released from the pump, is directed via a catheter to a stereotaxically placed cannula for infusion into the cerebroventricular space, as described herein.

For the methods of the invention, the therapeutically effective amount or dose can be estimated initially from cell culture assays. Then, the dosage can be formulated for use in animal models so as to achieve a circulating concentration range that includes the IC₅₀ as determined in cell culture. Such information can then be used to more accurately determine useful doses in humans.

Toxicity and therapeutic effective amount of the compounds described herein can be determined by standard pharmaceutical procedures in cell cultures or experimental animals, e.g., by determining the IC₅₀ and the LD₅₀. The data obtained from these cell culture assays and animal studies can be used in formulating a range of dosage for use in humans. The dosage can vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval can be adjusted individually to provide plasma levels of the leukocyte delivery agent triggering a response. These plasma levels are referred to as minimal effective concentrations (MECs). The MEC will vary for each compound but can be estimated from in vitro data. Dosages necessary to achieve the MEC will depend on individual characteristics and route of administration.

Dosage intervals can also be determined using MEC value. Compounds should be administered using a regimen that maintains plasma levels above the MEC for 10-90% of the time, preferably between 30-90% and most preferably between 50-90%.

In cases of local administration or selective uptake; the effective local concentration of the leukocyte delivery agent can not be related to plasma concentration. In such cases, other procedures known in the art can be employed to determine the correct dosage amount and interval.

The amount of the pharmaceutical composition of the leukocyte delivery agent of the present invention administered will, of course, be dependent on the subject being treated, the severity of the affliction, the manner of administration, the judgment of the prescribing physician, etc.

The pharmaceutical composition comprising the leukocyte delivery agent can, if desired, be presented in a suitable container (e.g., a pack or dispenser device), such as an FDA approved kit, which can contain one or more unit dosage forms containing the carrier portion containing the targeting and immune response triggering portions.

The method can further comprise administering to a subject a second therapy, wherein the second therapy is therapy for the treatment of CNS disorders, or an anti-cancer therapy, for example an anti-angiogenic therapy, chemotherapy, immunotherapy, surgery, radiotherapy, immunosuppresive agents, or gene therapy with a therapeutic polynucleotide. The second therapy can be administered to the subject before, during, after or a combination thereof relative to the administration of the leukocyte delivery agent as disclosed herein. Anti-cancer therapies are well known in the art and are encompassed for use in the methods of the present invention. Chemotherapy includes, but is not limited to an alkylating agent, mitotic inhibitor, antibiotic, or antimetabolite, anti-angliogenic agents eyc. The chemotherapy can comprise administration of CPT-11, temozolomide, or a platin compound. Radiotherapy can include, for example, x-ray irradiation, w-irradiation, γ-irradiation, or microwaves.

Pharmaceutical compositions of the leukocyte delivery agents or endothelial delivery agents as disclosed herein can be administered by any convenient route, including parenteral, enteral, mucosal, topical, e.g., subcutaneous, intravenous, topical, intramuscular, intraperitoneal, transdermal, rectal, vaginal, intranasal or intraocular. In one embodiment, the lipid particles of the present invention are not topically administered. In one embodiment, the delivery is by oral administration of the particle formulation. In one embodiment, the delivery is by intranasal administration of the particle formulation, especially for use in therapy of the brain and related organs (e.g., meninges and spinal cord) that seeks to bypass the blood-brain barrier (BBB). Along these lines, intraocular administration is also possible. In another embodiment, the delivery means is by intravenous (i.v.) administration of the particle formulation, which is especially advantageous when a longer-lasting i.v. formulation is desired. Suitable formulations can be found in Remington's Pharmaceutical Sciences, 16th and 18th Eds., Mack Publishing, Easton, Pa. (1980 and 1990), and Introduction to Pharmaceutical Dosage Forms, 4th Edition, Lea & Febiger, Philadelphia (1985), each of which is incorporated herein by reference.

It is still another object of the present invention to provide gene delivery using leukocyte delivery agents or endothelial delivery agents as disclosed herein as the gene delivery materials. For example, CD1, CD25 or CD69 genes or Ku70 gene, or homologues thereof can be targeted and delivered to leukocytes for therapeutic purposes etc.

In some embodiments, the present invention can be defined in any one of the following paragraphs:

1. A composition for delivering at least one insoluble agent and at least one soluble agent to a target cell comprising: (a) a targeting moiety that selectively binds one or more cell surface markers on the surface of the target cell; (b) a carrier particle associated with the targeting moiety, wherein the carrier particle has a lipid phase and an aqueous phase; (c) an insoluble agent entrapped in the lipid phase of the carrier particle; and (d) a soluble agent entrapped in the aqueous phase of the carrier particle
2. The composition of paragraph 1, wherein herein the targeting moiety comprises an antibody or integrin ligand, or functional fragments or variants thereof.
3. The composition of paragraph 1, wherein the targeting moiety comprises a scFv, an IgG, Fab′, F(ab′)2, or a recombinant bivalent scFv, or fragments thereof.
4. The composition of paragraph 1, wherein the carrier particle comprises a liposome or other lipid or non-lipid carrier or a functional fragment thereof.
5. The composition of paragraph 4, wherein the liposome is unilamellar with a first layer comprising glycosaminoglycan hyaluronan (HA) covalently linked to phosphatidylethanolamine therein, and a second layer comprising specific antibodies covalently attached to the HA of the first layer.
6. The composition of paragraph 1, wherein the insoluble agent is selected from the group consisting of a lipophilic RNAi, paclitaxel, platinum-based drugs, anthracyclines, mitomycin C, compounds of the quinolone family of synthetic antibacterial compounds, enoxacin, ciprofloxacin, ofloxacin, norfloxacin, and difloxacin and combinations and analogues thereof.
7. The composition of paragraph 1, wherein the soluble agent is selected from the group consisting of an RNA interference (RNAi) molecule, a small molecule, a polypeptide, antibody or analogues, variants or functional fragments thereof.
8. The composition of paragraph 7, wherein the RNA interference molecule is selected from the group consisting of siRNA, dsRNA, stRNA, shRNA, miRNA, and combinations thereof.
9. The composition of paragraph 1, wherein the target cell is a mammalian cell.
10. The composition of paragraph 1, wherein the target cell is a human cell.
11. A leukocyte-selective delivery agent comprising: (a) a targeting moiety that selectively binds one or more integrins on the surface of a leukocyte, wherein the integrin is in an active conformation; (b) a carrier particle associated with the targeting moiety, wherein the carrier particle having a lipid phase and an aqueous phase; (c) an insoluble agent entrapped in the lipid phase of the carrier particle; and/or (d) a soluble agent entrapped in the aqueous phase of the carrier particle
12. A leukocyte-selective delivery agent comprising; (a) a targeting moiety that selectively binds one or more integrins on the surface of a leukocyte; (b) a carrier particle associated with the targeting moiety, wherein the carrier particle having a lipid phase and an aqueous phase; (c) an insoluble agent entrapped in the lipid phase of the carrier particle; and/or (d) a soluble agent entrapped in the aqueous phase of the carrier particle.
13. The delivery agent of paragraph 12, which is further selective for activated leukocytes, wherein the targeting moiety selectively binds the leukocyte specific integrin in its activated conformation.
14. The delivery agent of paragraphs 11 or 12 wherein the integrin is selected from the group consisting of LFA-1 (αLβ2), Mac-1 (αMβ2), p150.95 (αXβ2), αDβ2, VLA-4 (α4β1), and β₇ OAP and αEβ7).
15. The delivery agent of paragraphs 11 or 12 wherein the integrin can bind an integrin ligand selected from the group consisting of ICAM-1, ICAM-2, ICAM-3, VCAM-1, MAdCAM-1, E-cadherin, JAM-1, JAM-2 and JAM-3.
16. The delivery agent of paragraph 15, wherein the integrin is LFA-1 and the targeting moiety comprises an antibody or functional fragment thereof, which binds to the locked open I domain of LFA-1, or binds to the leg domain of the α2 subunit of LFA-1 (αLβ2) or integrin β₇.
17. The delivery agent of paragraph 11 or 12, wherein the targeting moiety comprises an antibody or integrin ligand, or functional fragments or variants thereof.
18. The delivery agent of paragraph 17 wherein the targeting moiety comprises a scFv, an IgG, Fab′, F(ab′)2, or a recombinant bivalent scFv, or fragments thereof.
19. The delivery agent of paragraph 12, wherein the targeting moiety comprises an antibody or functional fragment thereof, which binds non-selectively to low affinity and high affinity LFA-1, Mac-1 and integrin P7.
20. The delivery agent of paragraph 11 or 12, wherein the carrier particle comprises a liposome or other lipid or non-lipid carrier or a functional fragment thereof.
21. The delivery agent of paragraph 20 wherein the lipo some is unilamellar with a first layer comprising glycosaminoglycan hyaluronan (HA) covalently linked to phosphatidylethanolamine therein, and a second layer comprising specific antibodies covalently attached to the HA of the first layer.
22. The delivery agent of paragraph 11 or 12, wherein the agent comprises one or more agents selected from the group consisting of an RNA interference (RNAi) molecule, a small molecule, a polypeptide, lipophilic agent, hydrophobic agent, antibody or analogues, variants or functional fragments thereof.
23. The delivery agent of paragraph 22 wherein the RNA interference molecule is selected from the group consisting of siRNA, dsRNA, stRNA, shRNA, miRNA, and combinations thereof.
24. The delivery agent of paragraph 23 wherein the RNAi molecule functions in gene silencing of Bcl-2, AKT, Mc1-1, HSP90, Histone d-acetylase 6 (HDAC6), CCR5, ku70, CD4, CDK1, CDK2, CDK3, CDK4, CDK5, CDK6, CDK7, CDK8, CDK9, CDK10, CDK11, cyclin A, cyclin B, cyclin C, cyclin D, or cyclin-D1.
25. The delivery agent of paragraph 22 wherein the hydrophobic agent is selected from the group consisting of a lipophilic RNAi, paclitaxel, platinum-based drugs, anthracyclines, mitomycin C compounds of the quinolone family of synthetic antibacterial compounds, enoxacin, ciprofloxacin, ofloxacin, norfloxacin, and difloxacin and combinations and analogues thereof.
26. A method for delivery of an agent to a leukocyte comprising; (a) administering a biological sample comprising leukocytes a leukocyte-selective delivery agent of paragraph 11 or paragraph 12, wherein the leukocyte-selective delivery agent comprises: (i) a targeting moiety that selectively binds one or more integrins on the surface of a leukocyte, wherein the integrin in an activated conformation;
(ii) a carrier particle associated with the targeting moiety, wherein the carrier particle has a lipid phase and a aqueous phase; wherein a lipophilic agent or hydrophobic agent is associated with the lipid phase of the carrier particle and/or a hydrophilic agent is associated with the aqueous phase of the carrier particle, (b) contacting the leukocyte delivery agent with a leukocyte, wherein contacting the leukocyte delivery agent with the leukocyte delivers the agents to the leukocyte.
27. The method of paragraph 26, wherein the leukocyte is an activated leukocyte.
28. The method of paragraph 26, wherein the biological sample is present in a subject.
29. The method of paragraph 26, wherein the biological sample is obtained from a subject.
30. The method of paragraphs 26 and 29, further comprising administering the leukocytes of step (b) to a subject, wherein the leukocytes have had agents delivered by the leukocyte-selective delivery agent.
31. The method of paragraph 26, wherein the biological sample is ex vivo.
32. The method of paragraph 26, wherein the biological sample is in vivo.
33. The method of paragraph 26, wherein the biological sample is in vitro.
34. The method of paragraphs 28 or 29, wherein the subject is a human.
35. The method of paragraphs 26 or 27, wherein the leukocyte has inappropriate leukocyte activation.
36. The method of paragraphs 28 or 29, wherein the subject has inappropriate leukocyte activation.
37. The method of paragraph 26, wherein the integrin is LFA-1 and the targeting moiety comprises an antibody, or functional fragment thereof, wherein the targeting moiety binds to LFA-1 in a locked open I domain configuration with higher affinity as compared to LFA-1 in a locked closed I domain configuration, or the targeting moiety binds to the leg domain of the α2 subunit of LFA-1 (αLβ2).
38. The method of paragraph 26 wherein the integrin is β₇ and the targeting moiety comprises an antibody or functional fragment thereof, which binds to integrin β₇.
39. A method for delivery of agents to a leukocyte present in a subject, comprising; (a) administering to a subject leukocyte-selective delivery agent of paragraph 1 or paragraph 2, wherein the leukocyte-selective delivery agent comprises: (i) a targeting moiety that selectively binds one or more integrins on the surface of a leukocyte, wherein the integrin in an activated conformation; (ii) a carrier particle associated with the targeting moiety, wherein the carrier particle has a lipid phase and a aqueous phase; wherein a lipophilic agent or hydrophobic agent is associated with the lipid phase of the carrier particle and/or a hydrophilic agent is associated with the aqueous phase of the carrier particle; and (b) contacting the leukocyte delivery agent with a leukocyte, wherein contacting the leukocyte delivery agent with the leukocyte delivers the agents to the leukocyte.
40. The method of paragraph 39, wherein the leukocyte-selective delivery agent is further selective for activated leukocytes.
41. The method of paragraphs 39 and 40, wherein the wherein the targeting moiety binds with higher affinity to integrins in an activated conformation as compared to integrins in an inactive conformation.
42. The method of paragraphs 39 or 41, wherein the integrin is selected from the group consisting of LFA-1, Mac-1, and β₇.
43. The method of paragraph 39, wherein the integrin is LFA-1 and the targeting moiety comprises an antibody or functional fragment thereof, wherein the targeting moiety binds to LFA-1 in a locked open I domain configuration with higher affinity as compared to LFA-1 in a locked closed I domain configuration, or the targeting moiety binds to the leg domain of the α2 subunit of LFA-1 (αLβ2).
44. The method of paragraph 39, wherein the integrin is β₇ and the targeting moiety comprises an antibody or functional fragment thereof, which binds to the β₇ integrin.
45. The method of paragraph 39, wherein the targeting moiety comprises an antibody or functional fragment thereof.
46. The method of paragraph 45, wherein the targeting moiety comprises an scFv, IgG, Fab′, F(ab′)2, and a recombinant bivalent scFv.
47. The method of paragraph 39, wherein the targeting moiety comprises an antibody or functional fragment thereof, which binds non-selectively to both low affinity and high affinity LFA-1 and to Integrin β₇.
48. The method of paragraph 39, wherein the carrier particle comprises a liposome or a lipid particle or a non-lipid particle and a functional fragment thereof.
49. The method of paragraph 38, wherein the liposome is unilamellar with a first layer comprising glycosaminoglycan hyaluronan (HA) covalently linked to phosphatidylethanolamine therein, and a second layer comprising specific antibodies covalently attached to the HA of the first layer.
50. The method of paragraphs 39, wherein the agent comprises one or more agents selected from the group consisting of an RNA interference (RNAi) molecule, a small molecule, a polypeptide, a hydrophobic agent, a poorly soluble drug and an antibody or functional fragment thereof.
51. The method of paragraph 50, wherein the RNA interference molecule is selected from the group consisting of siRNA, dsRNA, stRNA, shRNA, miRNA, and combinations thereof.
52. The method of paragraph 51, wherein the siRNA comprises CCR5-siRNA, ku70-siRNA, CD4-siRNA, Bcl-2-siRNA, AKT-siRNA, Mcl-1-siRNA, HSP90-siRNA, Histone d-acetylase 6-siRNA (HDAC6), CD4-siRNA, CDK1-siRNA, CDK2-siRNA, CDK3-siRNA, CDK4-siRNA, CDK5-siRNA, CDK6-siRNA, CDK7-siRNA, CDK8-siRNA, CDK9-siRNA, CDK10-siRNA, CDK11-siRNA, cyclin A-siRNA, cyclin B-siRNA, cyclin C-siRNA, cyclin D-siRNA, or cyclin-D1-siRNA.
53. The method of paragraph 50, wherein the hydrophobic agent is selected from the group consisting of a lipophilic RNAi, paclitaxel, platinum-based drugs, anthracyclines, mitomycin C compounds of the quinolone family of synthetic antibacterial compounds, enoxacin, ciprofloxacin, ofloxacin, norfloxacin, and difloxacin and combinations and analogues thereof.
54. A system for delivering an agent to a leukocyte, the system comprising; (a) a targeting moiety that selectively binds one or more integrins on the surface of a leukocyte; (b) a carrier particle associated with the targeting moiety, wherein the carrier particle having a lipid phase and an aqueous phase; wherein a lipophilic agent or hydrophobic agent can be entrapped in the lipid phase of the carrier particle and/or a hydrophilic agent can be entrapped in the aqueous phase of the carrier particle.
55. The system of paragraph 54, wherein the integrin is selected from the group consisting of LFA-1 (αLβ2), Mac-1 (αMβ2), p150.95 (αXβ2), VLA-4 (α4β1), and β₇ (α4β7 and αEβ7).
56. The system of paragraph 54, wherein the integrin can bind an integrin ligand selected from the group consisting of ICAM-1, ICAM-2, ICAM-3, VCAM-1, MAdCAM-1, E-Cadherin, JAM-1, JAM-2 and JAM-3.
57. The system of paragraph 54, wherein the integrin is LFA-1 and the targeting moiety comprises an antibody or functional fragment thereof, which binds to the locked open I domain of LFA-1, or binds to the leg domain of the α2 subunit of LFA-1 (αLβ2) or integrin β₇.
58. The system of paragraph 54, wherein the targeting moiety comprises an antibody or integrin ligand, or functional fragments or variants thereof.
59. The system of paragraph 54, wherein the targeting moiety comprises a scFv, an IgG, Fab′, F(ab′)2, or a recombinant bivalent scFv, or fragments thereof.
60. The system of paragraph 54, wherein the targeting moiety comprises an antibody or functional fragment thereof, which binds non-selectively to low affinity and high affinity LFA-1, Mac-1 and integrin β₇.
61. The system of paragraph 54, wherein the carrier particle comprises a liposothe or other lipid or non-lipid carrier or a functional fragment thereof.
62. The system of paragraph 54, wherein the liposome is unilamellar with a first layer comprising glycosaminoglycan hyaluronan (HA) covalently linked to phosphatidylethanolamine therein, and a second layer comprising specific antibodies covalently attached to the HA of the first layer.
63. The system of paragraph 54, wherein the hydrophilic agent comprises one or more agents selected from the group consisting of an RNA interference (RNAi) molecule, a small molecule, a polypeptide, antibody or analogues, variants or functional fragments thereof.
64. The system of paragraph 54, wherein the RNA interference molecule is selected from the group consisting of siRNA, dsRNA, stRNA, shRNA, miRNA, and combinations thereof.
65. An endothelial cell-selective delivery agent comprising, (a) a targeting moiety that selectively binds one or more integrin ligands on the surface of a endothelial cell; (b) a carrier particle associated with the targeting moiety, wherein the carrier particle having a lipid phase and an aqueous phase; (c) an insoluble agent entrapped in the lipid phase of the carrier particle; and/or (d) a soluble agent entrapped in the aqueous phase of the carrier particle
66. The delivery agent of paragraph 65, wherein the integrin ligand is selected from the group consisting of ICAM-1, ICAM-2, ICAM-3, VCAM-1, MAdCAM-1, E-Cadherin, JAM-1, JAM-2 and JAM-3.
67. The delivery agent of paragraph 65, wherein the integrin ligand can bind to an integrin present on the surface of leukocytes.
68. The delivery agent of paragraphs 65 or 67, wherein the integrin ligand can bind to integrins selected from the group consisting of LFA-1 (αLβ2), Mac-1 (αMβ2), p150.95 (αXβ2), αDβ2, VLA-4 (α4β1), and β₇ (α4β7 and αEβ7).
69. The delivery agent of paragraph 65, wherein the targeting moiety comprises an antibody or integrin, or functional fragments or variants thereof.
70. The delivery agent of paragraph 69, wherein the targeting moiety comprises a scFv, an IgG, Fab′, F(ab′)2, or a recombinant bivalent scFv, or fragments thereof.
71. The delivery agent of paragraph 65, wherein the carrier particle comprises a liposome or other lipid or non-lipid carrier or a functional fragment thereof.
72. The delivery agent of paragraph 61, wherein the liposome is unilamellar with a first layer comprising glycosaminoglycan hyaluronan (HA) covalently linked to phosphatidylethanolamine therein, and a second layer comprising specific antibodies covalently attached to the HA of the first layer.
73. The delivery agent of paragraph 65, wherein the agent comprises one or more agents selected from the group consisting of an RNA interference (RNAi) molecule, a small molecule, a polypeptide, lipophilic agent, hydrophobic agent, antibody or analogues, variants or functional fragments thereof.
74. The delivery agent of paragraph 73, wherein the RNA interference molecule is selected from the group consisting of siRNA, dsRNA, stRNA, shRNA, miRNA, and combinations thereof.
75. The delivery agent of paragraph 74, wherein the RNAi molecule functions in gene silencing VEGF, and/or other angiogenesis genes.
76. The delivery agent of paragraph 73, wherein the hydrophobic agent is selected from the group consisting of a lipophilic RNAi, paclitaxel, platinum-based drugs, anthracyclines, mitomycin C, compounds of the quinolone family of synthetic antibacterial compounds, enoxacin, ciprofloxacin, ofloxacin, norfloxacin, and difloxacin and combinations and analogues thereof.
77. A method for delivery of an agent to an endothelial cell comprising; (a) administering to endothelial cells an endothelial cell-selective delivery agent of paragraph 65, wherein the leukocyte-selective delivery agent comprises: (i) a targeting moiety that selectively binds one or more integrin ligands on the surface of an endothelial cell; (ii) a carrier particle associated with the targeting moiety, wherein the carrier particle has a lipid phase and a aqueous phase; wherein a lipophilic agent or hydrophobic agent is associated with the lipid phase of the carrier particle and/or a hydrophilic agent is associated with the aqueous phase of the carrier particle, (b) contacting the endothelial cell-delivery agent with an endothelial cell, wherein contacting the endothelial cell delivery agent with the endothelial cell delivers the agents to the endothelial cell.
78. The method of paragraph 77, wherein administration is to a subject.
79. The method of paragraph 77, wherein administration is to a biological sample.
80. The method of paragraph 79, wherein the biological sample is obtained from a subject.
81. The method of paragraphs 77 and 79, further comprising administering the endothelial cells of step (b) to a subject, wherein the 1 endothelial cells have had agents delivered by the endothelial cell-selective delivery agent.
82. The method of paragraph 79, wherein the biological sample is ex vivo.
83. The method of paragraph 79, wherein the biological sample is in vivo.
84. The method of paragraph 79, wherein the biological sample is in vitro.
85. The method of paragraphs 78 or 80, wherein the subject is a human.
86. The method of paragraph 77, wherein the endothelial cell has inappropriate endothelial cell proliferation.
87. The method of paragraphs 78, 80 or 85, wherein the subject has inappropriate endothelial cell proliferation.
88. A system for delivering an agent to an endothelial cell, the system comprising; (a) a targeting moiety that selectively binds one or more integrin ligands on the surface of an endothelial cell; (b) a carrier particle associated with the targeting moiety, wherein the carrier particle having a lipid phase and an aqueous phase; wherein a lipophilic agent or hydrophobic agent can be entrapped in the lipid phase of the carrier particle and/or a hydrophilic agent can be entrapped in the aqueous phase of the carrier particle.
89. The system of paragraph 88, wherein the integrin ligand is selected from the group consisting of ICAM-1, ICAM-2, ICAM-3, VCAM-1, MAdCAM-1, E-cadherin, JAM-1, JAM-2 and JAM-3.
90. The system of paragraph 88, wherein the integrin ligand can bind to an integrin present on the surface of leukocytes.
91. The system of paragraphs 88 and 90, wherein the integrin ligand can bind to integrins selected from the group consisting of LFA-1 (αLβ2), Mac-1 (αMβ2), p150.95 (αXβ2), αDβ2, VLA-4 (α4β1), and β₇ (α4β7 and αEβ7).
92. The system of paragraph 88, wherein the targeting moiety comprises an antibody or integrin, or functional fragments or variants thereof.
93. The system of paragraph 92, wherein the targeting moiety comprises a scFv, an IgG, Fab′, F(ab′)2, or a recombinant bivalent scFv, or fragments thereof.
94. The system of paragraph 88, wherein the carrier particle comprises a liposome or other lipid or non-lipid carrier or a functional fragment thereof.
95. The system of paragraph 94, wherein the liposome is unilamellar with a first layer comprising glycosaminoglycan hyaluronan (HA) covalently linked to phosphatidylethanolamine therein, and a second layer comprising specific antibodies covalently attached to the HA of the first layer.
96. The system of paragraph 88, wherein the agent comprises one or more agents selected from the group consisting of an RNA interference (RNAi) molecule, a small molecule, a polypeptide, lipophilic agent, hydrophobic agent, antibody or analogues, variants or functional fragments thereof.
97. The system of paragraph 96, wherein the RNA interference molecule is selected from the group consisting of siRNA, dsRNA, stRNA, shRNA, miRNA, and combinations thereof.
98. The system of paragraph 97, wherein the RNAi molecule functions in gene silencing VEGF, and/or other angiogenesis genes.
99. The system of paragraph 96, wherein the hydrophobic agent is selected from the group consisting of a lipophilic RNAi, paclitaxel, platinum-based drugs, anthracyclines, mitomycin C, compounds of the quinolone family of synthetic antibacterial compounds, enoxacin, ciprofloxacin, ofloxacin, norfloxacin, and difloxacin and combinations and analogues thereof.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages may mean±1%.

### EXAMPLES

The examples presented herein relates to methods to deliver agents to leukocytes, by associating a targeting agent to a carrier particle, where the carrier particle comprises an agent, and the targeting agent binds to, or has affinity for an integrin present on the surface of a leukocyte. In alternative embodiments, the present invention relates to methods to deliver agents to endothelial cells by associating a targeting agent to a carrier particle, where the carrier particle comprises an agent, and the targeting agent binds to, or has affinity for integrin ligands present on the surface of endothelial cells. Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

### METHODS

Construction of Particles.

Particles were prepared by the lipid-film method and composed of egg phosphatidylcholine (PC); 1,2-Dipalmitoyl-sn-glycero-3-phosphoethanolamine (DPPE), and Cholesterol (Chol) at a molar ratio of 3:1:1 (Avanti Polar Lipids, Alabaster, Ala.). The lipids (25 mmol total lipid in each formulation) were dissolved in chloroform-methanol (3:1, volume/volume), evaporated to dryness under reduced pressure (70 mm Hg) at 37° C. water bath in a rotary evaporator (Buchi Rotavapor R-114 with a vacuum controller V-800). The lipid film was placed under high vacuum for 24 h to remove residual organic solvents. The hydration of thin films consisted of buffer alone (HEPES-buffered saline, 10 mM Hepes, 140 mM NaCl, pH 7.4; HBS) followed by extensive agitation using a vortex device and a 2 h incubation in a shaker bath at 37° C. Unilamellar vesicles (ULV) were obtained by extrusion of the Multilamellar vesicles (MLV), operating the extrusion device (Lipex™ Thermobarrel Extruder System, Northern lipids Inc., Vancouver, Canada) at room temperature and under nitrogen pressures of 400 to 600 psi. with circulating water bath to heat the extruder (above the lipid transition state temp. >60° C.) The extrusion was carried out in stages using progressively smaller pore-size polycarbonate membranes (1.0, 0.8, 0.4, 0.2, 0.1 μm) with several cycles per pore-size. The liposomes were extruded 10 times in the smallest filter. Lipid mass analysis was determined with a phosphate assay (Morrison, 1964) or by the incorporation of ³H-CHE (cholesterol derivative, PerkinElmer, Wellesley, Mass.).

Particle Diameter Measurements.

The diameter of the particles was measured on a Malvern Zetasizer nano ZS Zeta potential and Dynamic Light Scattering Instrument (Malvern Instruments Ltd., Southborough, Mass.) using the automatic algorithm mode and analyzed with the PCS 1.32a.

Surface Modification.

Hyaluronan modified-liposomes. 20 mg HA, Mw 835 KDa, intrinsic viscosity: 16 dL/g (Genzyme cooperation, Cambridge, Mass.) was dissolved in 10 mL double distilled water to a final concentration of 2 mg/mL and pre-activated by incubation with soluble carbodiimide (EDAC), at pH 4 (controlled by titration with HCl) for 2 h at 37° C. At the end of this step, the activated HA was added to DPPE-containing Small Unilamellar Vesicles (PC:Chol:DPPE) in 0.1M borate buffer to a final pH of 9.0 and incubated overnight at 37° C. At the end of the incubation, the liposomes were separated from excess reagents and by-products by centrifugation (135,000 g, 4° C., 1 hr) and repeated washings (all in HBS, pH 7.4). The final ratio of ligand to lipid was 75 μg HA/μmole lipid assayed by ³H-HA (ARC, Saint Louis, Mich.).

Coupling Monoclonal Antibodies to the Liposomal Surface.

L Labeling antibodies with fluorescence dyes. In order to detect the binding of mAb-coupled liposomes to cells via flow cytometry the inventors directly labeled the mAb prior to coupling them to the liposomal surface. Labeling was performed on purified antibodies FIB504.64 (rat anti-human/mouse) against integrin β₇ was purified from hybridoma; M17/4 (rat anti-mouse) against integrin α_{L} purified from hybridoma; rat IgG₂ₐ isotype control (BD Pharmigen); TS1/22 mouse anti-human against integrin α_{L} purified from hybridoma and mouse IgG1, isotype control. (BD Pharmigen); all dissolved in HBS pH 7.4 at a concentration of 1 mg/mL in a total volume of 1 mL. 1/10 volume of 1M NaHCO₃, pH 8.5 was added to the antibody solution. The mixture (antibody/HBS/NaHCO₃) was transferred to one vial of desiccated primary amine-reactive (succinimidyl esters) Cy3 dye (Amersham Bioscience, GE Healthcare, Pittsburgh, Pa.) or Alexa 488 dye (Invitrogen) and mixed well to dissolve the dye. The suspension, protected from light, was incubated at room temperature for 20 minutes. The reaction was quenched by adding 1/20 volume of 3M Tris, pH 7.2. Separation from excess dye was done using a desalting column washed with HBS. Assessment of labeling and concentration was done according to manufacture's guidelines.

II. Coupling mAb through HA spacer to the liposomal surface. 50 μL HA-modified SUV (PC:Chol: DPPE; 25 mmol total lipid, 75 μg HA/μmole lipid) were incubated with 200 μL of 400 mmol/L 1-(3-dimethylaminopropyl)-3-ethylcarbodimide hydrochloride (EDC) and 200 μL of 100 mmol/L N-hydroxysuccinimide (NHS) for 20 minutes at room temperature with gentle stirring. The resulting NHS-activated HA-nanoliposomes were covalently linked to 50 μL of a 5 mg/mL mAb purified from hybridoma (in HBS, pH 7.4) and incubated for 150 min at room temperature with gentle stirring followed by 10 μL 1M ethanolamine Hcl (pH 8.5) to block reactive residues. The resulting Immuno-nanoliposomes were purified by size exclusion column packed with sepharose CL-4B beads (Sigma-Aldrich, Saint Louis, Mich.) equilibrate with HBS, pH 7.4.

To asses coupling efficiency of the mAbs and densities on the surface of the nanoliposomes, a trace amount of ¹²⁵I-labeled FIB504.64 (rat mAb against integrin β₇) or Rat IgG₂ₐ, isotype control were added to the unlabeled mAb prior to coupling as previously reported (Sapra and Allen, 2004). Iodination of mAbs was done using Iodo-Gen iodination reagent (Pierce) according to the manufacturer's protocol.

A coupling efficiency of 75-80% was determined for the method of coupling. We made ensure that similar antibody densities (within ±10%) occurred at the surface of either type of immunoliposomes.

Incorporation of Hydrophilic Drugs

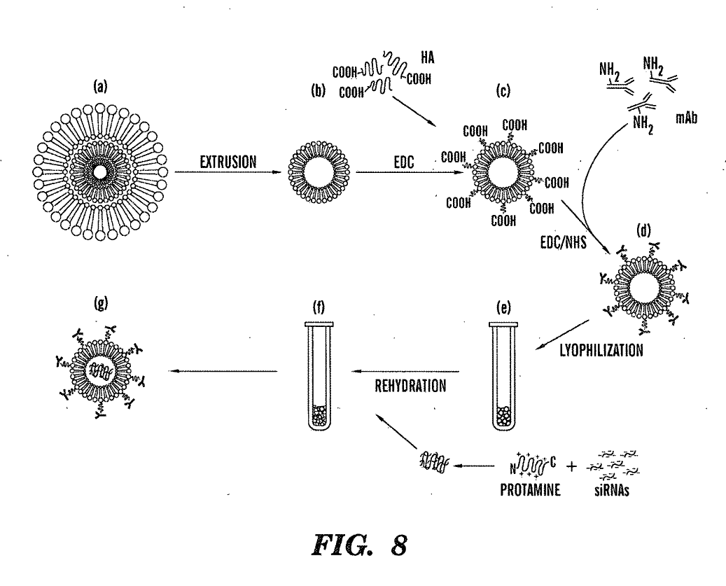

In order to incorporate hydrophilic drugs we used a method developed by us employing lyophilization and reconstitute process (see FIG. 8).

Lyophilization.

Lyophilization of liposome suspensions was performed on 0.5 mL aliquots. Samples were frozen for 4 hours at −80° C. and lyophilized for 48 hours. Reconstitution was to original volume using the hydrophilic drug of choice dissolved in distilled water. Encapsulation and release kinetics were calculated as previously described²⁻⁵.

Release Kinetics.

A suspension of nanoparticles (0.5-1.0 mL) was placed in a dialysis sac and the sac was immersed in a continuously-stirred receiver vessel, containing drug-free HBS, pH 7.4. Receiver to liposome-sample volume ratios were 10-16. At designated periods, the dialysis sac was transferred from one receiver vessel to another containing fresh drug-free buffer. Drug concentration was assayed in each dialysate and in the sac, at the beginning and end of each experiment.

In order to obtain a quantitative evaluation of drug release, experimental data were analyzed according to a previously-derived multi-pool kinetic model (Margalit R., 1991). For this model, the relationship between time (the free variable) and the dependant variable f(t)—the cumulative drug released into the dialysate at time t, normalized to the total drug in the system at time=0—is expressed in equation (1), below

$$f ( t ) = {\sum }_{j = 1}^{n} {f}_{j} ( 1 - {exp}^{- {k}_{j} t} ) \qquad ( equation 1 )$$

where fⱼ is the fraction of the total drug in the system occupying the jth pool at time=0, and kⱼ is the rate constant for drug diffusion from the j'th pool.

Encapsulation Efficiency.

Defined as the ratio of entrapped drug to the total drug in the system, encapsulation efficiency can be determined by two independent methods: (1) By centrifugation. Samples of the complete liposome preparation, containing both encapsulated and unencapsulated drug, were centrifuged as described above. The supernatant, containing the unencapsulated drug, is removed. The pellet, containing the liposomes with encapsulated drug, is resuspended in drug-free buffer. Drug is assayed in the supernatant and in the pellet, as well as in the complete preparation, from which the encapsulation efficiency and conservation of matter can be calculated. (2) From data analysis of efflux kinetics. As discussed above, data analysis yields the parameter fⱼ. When the efflux experiment is performed on samples from the complete liposome preparation, the magnitude of fⱼ for the pool of encapsulated drug is also the efficiency of encapsulation.

Incorporation of Lipophilic Drugs.

The incorporation of lipophilic drugs was done in the course of particles preparation.

Briefly, when an insoluble drug was used the inventors dissolved the drug with the lipids. The inventors then continued as described above for the preparation of the particles. Insoluble drugs such as Taxol had tagged with tritium, which the inventors could assay at each step by radioactivity.

Co-Encapsulation of Hydrophilic and Lipophilic Drugs.

Co-encapsulation of drugs was achieved by entrapping first the lipophilic drugs in the lipids then make the particles and in the end of the process lyophilized the particles. Reconstitute with the hydrophilic drugs was made as describe above. FIG. 8 illustrates the co-entrapment method in the targeted nanoparticles.

Hydrophilic Drugs.

Preparation of siRNAs.

siRNAs from Dharmacon were deprotected and annealed according to the manufacturer's instructions. Four Ku70-siRNAs were used in an equimolar ratio as previously reported (Peer et al., 2007). Cyclin-D1-siRNAs were used in an equimolar ratio. Cyclin-D1-siRNAs Sequences were:

| (SEQ ID NO: 1) | |
|---|---|
| ACACCAAUCUCCUCAACGAUU | (sense # 1; |
| SEQ ID NO: 2) | |
| 5′-PUCGUUGAGGAGAUUGGUGUUU; | (antisense # 1; |
| SEQ ID NO: 3) | |
| GCAUGUUCGUGGCCUCUAAUU; | (sense # 2; |
| SEQ ID NO: 4) | |
| 5′-PUUAGAGGCCACGAACAUGCUU; | (antisense # 2; |
| SEQ ID NO: 5) | |
| GCCGAGAAGUUGUGCACUUUU; | (sense # 3; |
| SEQ ID NO: 6) | |
| 5′-PAGAUGCACAACUUCUCGGCUU; | (antisense # 3; |
| SEQ ID NO: 7) | |
| GCACUUUCUUUCCAGAGUCUU; | (sense # 4; |
| SEQ ID NO: 8) | |
| 5′-PGACUCUGGAAAGAAAGUGCUU. | (antisense # 4; |

siRNAs Entrapment in the Particles.

siRNAs were mixed with full length recombinant protamine (Abnova, Taipei City, Taiwan) in a 5:1 molar ratio, in DEPC-treated water (Ambion Inc., Austin, Tex.) and were pre-incubated for 30 min at RT to form a complex as previously reported (Peer et al., 2007), and added to the dry powder for entrapment inside the Immunonanoparticles as previously shown with other drugs (Peer and Margalit, 2000). siRNAs entrapment was determined using a RiboGreen assay (Molecular Probes, (Invitrogen)), comparing fluorescence in the presence and absence of Triton X-100.

Lipophilic Drug:

Paclitaxel (sigma) was added to the lipids at 3% mole (with respect to the phospholipids) and dissolved in 100% ethanol. Paclitaxel was assayed using a trace of ³H-Paclitaxel (American Radiolabeled Chemicals, Inc.; specific activity 60 Ci/mmol; conc. 1 mCi/mL).

In Vitro Treatment of Targeted Nanoparticles Entrapping Both Hydrophilic (siRNAs) and Lipophilic (Paclitaxel).

Cells (K562 expressing integrin LFA-1) were plated at a density of 2×10⁵ cells/well in 200 μL in quadruplicate an hour prior to experiment. Next, several formulations (siRNAs amounts: 1 nmol; paclitaxel conc. 50 nM) were given to cells for 4 hours, followed by washing and re-plating of the cells with fresh media and culturing the cells for additional 48 hours. The end point included a cell viability assay (MTT) normalized to untreated cells.

Interferon Assay.

Spleenocytes (1×10⁶ cells/ml) were mock treated or treated with β₇ It-sNP and luciferase-siRNA (1000 pmol) or 5 μg/ml poly (I:C). After 48 h RNA was isolated and analyzed by quantitative RT-PCR for induction of IFN or interferon responsive genes as described below.

Quantitative RT-PCR.

Total RNA (1 μg) isolated using RNeasy RNA isolation kit (Qiagen) or for tissues, RNAlater RNA stabilization reagent was used followed by RNeasy fibrous tissue mini kit (Qiagen) and reverse transcribed using Superscript III (Invitrogen) and random hexamers, according to the manufacturer's protocol. Real-time quantitative PCR was performed on 0.2 μl of cDNA or a comparable amount of RNA with no reverse transcriptase, using Platinum Taq Polymerase (Invitrogen) and a Biorad iCycler. SYBR green (Molecular Probes) was used to detect PCR products. All reactions were done in a 25 μl reaction volume in triplicate. Primers for mouse GAPDH, STAT1, OAS1 and INF β were previously described (Song et al., 2005). The following primer pairs have been used: IL-12p40: Forward: 5′-CTCACATCTGCTGCTCCACAAG-3′ (SEQ ID NO:9); Reverse: 5′-AATTTGGTGCTTCACACTTCAGG-3′ (SEQ ID NO:10); TNF α: Forward 5′-CCTGTAGCCCACGTCGTAGC-3′ (SEQ ID NO:11), Reverse 5′-TTGACCTCAGCGCTGAGTTG-3′ (SEQ ID NO:12); Cyclin D1: Forward 5′-CTT CCT CTC CAA AAT GCC AG-3′ (SEQ ID NO:13), Reverse 5′-AGA GAT GGA AGG GGG AAA GA-3′ (SEQ ID NO:14). PCR parameters consisted of 5 min of Taq activation at 95° C., followed by 40 cycles of PCR at 95° C.×20 sec, 60° C.×30 sec, and 69° C.×20 sec. Standard curves were generated and the relative amount of target gene mRNA was normalized to GAPDH mRNA. Specificity was verified by melt curve analysis and agarose gel electrophoresis.

Cell Isolation and Flow Cytometry

Mononuclear cells were isolated from SP, peripheral blood (PBL), PLN, MLN, PP, and IEL, as described (Park et al., 2003). Flow cytometry of cell surface antigens was performed as described (Peer et al., 2007). The following mAbs were used: FITC- or PE-conjugated mAbs to CD25, CD69 (BD Bioscience); FIB504.64 was grown and purified from hybridoma as previously described Rat IgG2a isotype control and FIB504.64 were pre-labeled with Alexa 488 using Alexa dye kit (Invitrogen), to probe the expression level of integrin β₇. In other cases, the pre-labeled mAbs were immobilized on HA-coated nanoliposomes to create a labeled version of β₇ It-sNP and Ig-sNP for microscopy studies. For intracellular staining of cyclin D1 and Ku70, cells were fixed and permeabilized with the Fix-and-Perm kit (Caltag Laboratories, Burlingame, Calif.), stained with 1 μg/ml goat anti-mouse cyclin D1 (Santa Cruz Biotechnology) on ice for 30 min, and counter-stained with FITC-conjugated rabbit anti-goat IgG (Zymed). Detection of Ku70 expression was as described (Peer et al., 2007). Data were acquired and analyzed on FACScan or FACScalibur with CellQuest software (Becton Dickinson, Franklin Lakes, N.J.).

Image Acquisition and Processing.

Confocal imaging was performed using a Biorad Radiance 2000 Laser-scanning confocal system (Hercules, Calif.) with an Olympus BX50BWI microscope using an Olympus 100×LUMPlanFL 1.0 water-dipping objective. Image acquisition was performed using Laserscan 2000 software and image processing was performed with Openlab 3.1.5 software (Improvision, Lexington, Mass.).

Colitis Model.

Mice were treated with dextran sodium sulphate (DSS), as previously described (Park et al., 2003). Briefly, colitis was induced by addition of 3.5% (wt/vol) for 9 days of DSS (MP Biomedicals, Inc.) in drinking water. Body weight was determined daily. Mice were sacrificed on day 10 and the entire colon was removed from cecum to anus, and colon length was measured as a marker of inflammation. Blood was obtained by cardiac puncture. Distal colon cross-sections were stained with Haematoxylin and Eosin for histologic examination and images were acquired using a Nikon Eclipse 80i Microscope and the SPOT software (Diagnostic Instruments, Inc.). Quantitative histopathologic grading of colitis severity was assessed as previously reported (Neurath et al., 2002).

Tissue Distribution Studies and Pharmacokinetic Analysis.

Radiolabeled β₇ It-sNP and Ig-sNP were prepared for short-term distribution studies by incorporation of 5 μCi/mg lipid of the non-exchangeable lipid label ³H-CHE as previously reported (Sapra and Allen, 2004). β₇ It-sNP and Ig-sNP were administered by lateral tail vein injection in healthy 8-week-old female C57BL/6 mice (Charles River Laboratories) and separately DSS-induced colitis 8-week-old female C57BL/6 mice. At 1, 6, and 12 h, blood was drown through the retroorbital vein, Plasma was isolated from whole blood by centrifugation at 3000×g for 5 min. Organ homogenates 10% (w/v) were prepared in water using a Polytron homogenizer (Brinkman Instruments, Mississauga, Ontario, Canada), and 500 μl of Solvable were added to 200 μl of either tissue homogenates or plasma. The solutions were then digested for 2 h at 60° C. After the vials cooled to room temperature, 500 of 200 mM EDTA were added before overnight bleaching with 200 μl of hydrogen peroxide [30% (v/v)]. The next day, 100 μl of 1 N HCl were added before 5 ml of Ultima Gold, and the samples were counted in a Beckman LS 6500 liquid scintillation counter for ³H.Blood correction factors were applied to correct for liposomes present in the blood volume of organs as previously described (Sapra and Allen, 2004). Results are expressed as percentage of injected drug or phospholipids (PL) present in blood at each time point.

In Vivo Gene Silencing.

Healthy or diseased (colitis) mice were injected intravenously with β₇ It-sNP and Ig-sNP entrapping 2.5 mg/Kg body siRNAs (Ku70, Cyclin-D1, Luciferase) as detailed in the figures.

### Example 1

Binding, Internalization and Silencing of siRNAs Delivered Via Integrin β₇-Targeted Stabilized Nanoparticles (β₇ It-sNP) to Cells Expressing Integrin β₇.

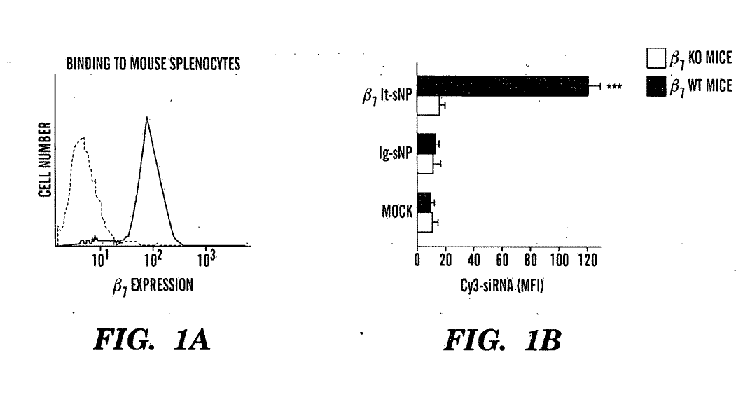

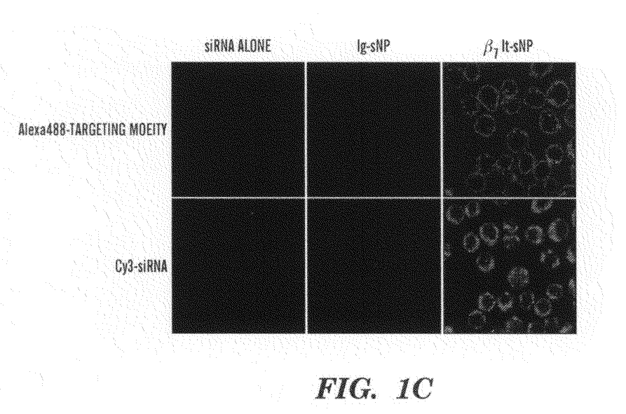

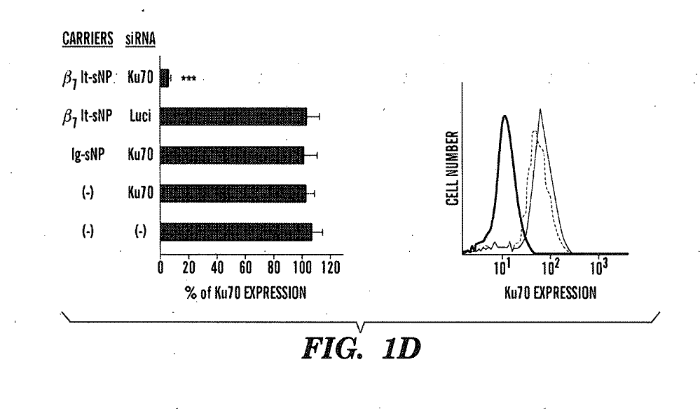

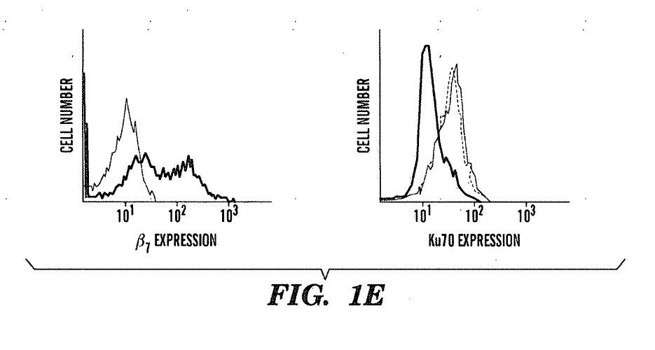

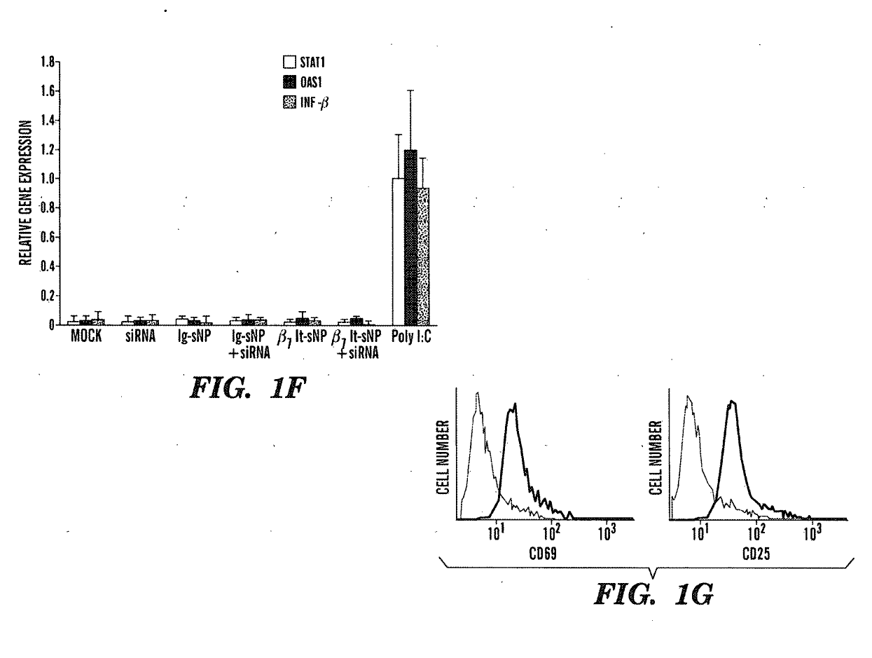

Rat antibody FIB504.64 against human integrin β₇ (cross-react with mouse) was immobilized on HA-coated nanoparticles β₇ It-sNP). β₇ It-sNP had a 120±20 nm mean diameter in solution. β₇ It-sNP was prepared as described in FIG. 8 (scheme 1) (without incorporating insoluble drugs). β₇ It-sNP bound to primary mouse splenocytes (expressing integrin β₇) (FIG. 1a). An isotype control immobilized on the surface of nanoparticles (Ig-sNP) did not bind to mouse primary splenocytes. Cy3-siRNA entrapped in β₇ It-sNP was selectively delivered to splenocytes isolated from wt mice but not to splenocytes isolated from β₇ knockout mice (FIG. 1b) demonstrating the selectivity to integrin β₇. Moreover, neither Ig-sNP entrapping Cy3-siRNA or Cy3-siRNA without any delivery system were capable to deliver fluorescent siRNA into neither kind of splenocytes (FIG. 1b). The inventors next used confocal microscopy to investigate the ability of Alexa488-labeled targeting moieties (FIB504.64 or isotype control) to bind and deliver Cy3-siRNA selectively to TK-1 cells (mouse T cells expressing the integrin β₇. Four hours after exposure of TK-1 cells to the fluorescently labeled β₇ It-sNP or Ig-sNP, Alexa488-β₇ It-sNP were distributed to both the plasma membrane and internal punctate structures, whereas Cy3-siRNA was predominantly intracellular, colocolized with the Alexa488-β₇ It-sNP (FIG. 1c). As expected, minimal Cy3-siRNA uptake was shown when they were delivered via Ig-sNP. Cy3-siRNA was barely detected when given without any delivery system to TK-1 cells.

The inventors next asked whether β₇ It-sNP delivering siRNAs could induce silencing of the ubiquitously expressed Ku70 gene (Peer et al., 2007) selectively to primary splenocytes (FIG. 1d1) and TK-1 cells (FIG. 1d2). Intracellular Ku70 staining was performed 48 h after treatment. Potent silencing was achieved at 1 nmol of siRNA when it was delivered via β₇ It-sNP, whereas Ku70-siRNA delivered via Ig-sNP or without any delivery system did not induced silencing. An irrelevant siRNA (luciferase) that was also delivered via β₇ It-sNP did not induce silencing, as expected. FIB504.64 is a rat anti-human mAb, therefore we tested the ability of β₇ It-sNP to efficiently bind and deliver Ku70-siRNAs to human PBMC. β₇ It-sNP efficiently bound to human PBMC (FIG. 1e1), whereas Ig-sNP did not. Efficient silencing of Ku70 in PMBC was achieved only when Ku70-siRNAs were delivered via β₇ It-sNP and not via Ig-sNP (FIG. 1e2). A possible unwanted off-target effect of immuno-nanoparticles-delivered siRNAs could be activation of Interferon (INF)-responsive genes (IRG) by activating cytosolic dsRNA-activated protein kinase PKR or by binding to Toll-like receptors 3, 7, and 8 that recognize RNA on the cell surface or in endosomes (Hornung et al., 2005). To test whether β₇ It-sNP entrapping siRNAs activate an IFN response, the inventors used quantitative RT-PCR to measure mRNA expression of INF-β, and two key genes 2′,5′-oligoadenylate syntase 1 (OAS1) and STAT1 (Peer et al., 2007). The inventors discovered IRG were not induced by the β₇ antibody or by the Rat isotype control antibody immobilized on the nanoparticles' surface but were induced by treatment with the known INF inducer poly (I:C) (FIG. 1e. Treatment with as much as 1 μM luciferase-siRNA delivered by either Ig-sNP or β₇ It-sNP did not induce an IFN response. Another possible undesired effect could be activation of cells that are targeted through these Immunonanoparticles. To determine whether the NP entrapping siRNAs cause lymphocyte activation, the inventors measured the induction of the early activation markers CD25 and CD69 on splenocytes cultured for 48 h with the NP entrapping luciferase-siRNA. Neither β₇ It-sNP entrapping siRNAs (FIG. 1g), nor Ig-sNP (data not shown) did not induced expression of CD69 or CD25, whereas activation with phytohemagglutinin (PHA) induced expression of both markers (FIG. 1g, and data no shown). Therefore, the inventors have determined that transducing lymphocytes with β₇ It-sNP does not activate them and does not trigger nonspecific INF response.

### Example 2

### In Vivo Delivery of siRNAs Via β₇ It-sNP

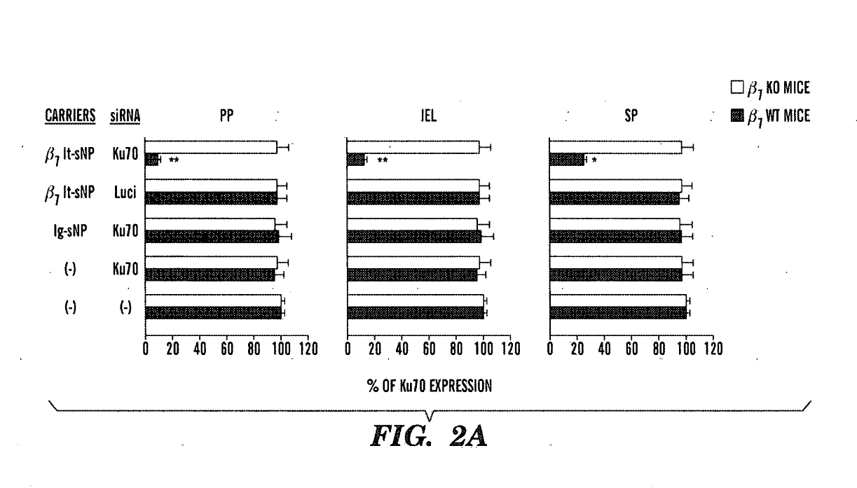

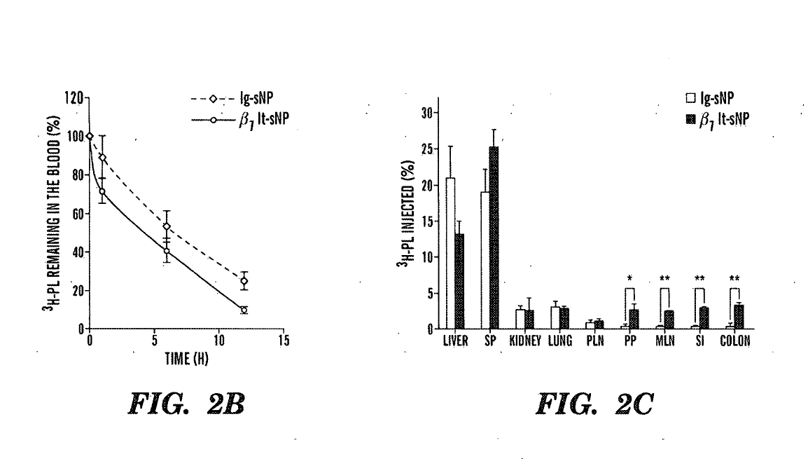

To investigate whether β₇ It-sNP could selectively deliver siRNAs in vivo to cells expressing integrin 137, a single intravenous injection of Ku70-siRNAs entrapped in β₇ It-sNP, or controls (Ku70-siRNAs in Ig-sNP, luciferase-siRNA in β₇ It-sNP as well as free (unprotected Ku70-siRNAs in PBS)) were given to C57BL/6 mice or β₇ knockout mice. Ku70 knocked down was shown in Payer's patches (PP), intraepithelial lymphocytes (IELs) and splenocytes (SP) only when delivered via β₇ It-sNP, but not when delivered via Ig-sNP (FIG. 2a). Luciferase-siRNA entrapped in β₇ It-sNP did not cause gene silencing, nor did unencapsulated Ku70-siRNA, as expected. Nonspecific uptake of Ku70-siRNAs delivered via Ig-sNP was negligible (FIG. 2a). Selectivity was demonstrated when β₇ It-sNP entrapping Ku70-siRNAs had no effect when injected intravenously to β₇ knockout mice (FIG. 2a). Insight into the pharmacokinetics and tissue profile distribution of β₇ It-sNP were achieve by pre-labeling the particles with a non-exchangeable [³H]CHE as previously described (Sapra and Allen, 2004). A single intravenous injection of Ig-sNP and separately of β₇ It-sNP labeled with a non-exchangeable [³H]CHE to the tail vein of healthy C57BL16 mice reveled that less than 12 hours post injection β₇ It-sNP were cleared from the circulation (FIG. 2b). β₇ It-sNP accumulated in tissues overexpressing integrin β₇ such as PP, MLN, small intestine (Si) and the colon in a highly specific manner (FIG. 2c). Control particles (Ig-sNP) were more circulating in the blood stream and were mainly accumulated, non-specifically, in the liver and spleen (FIG. 2c), which is a well documented phenomena of targeted and non-targeted nanoparticles (Demoy et al., 1999; Lasic et al., 1991; Papahadjopoulos et al., 1991; Peracchia et al., 1999).

### Example 3

### Targeting Cyclin-D1-siRNAs to Cells Expressing Integrin β₇

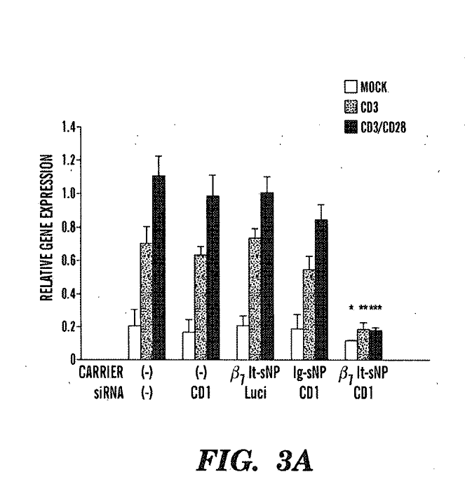

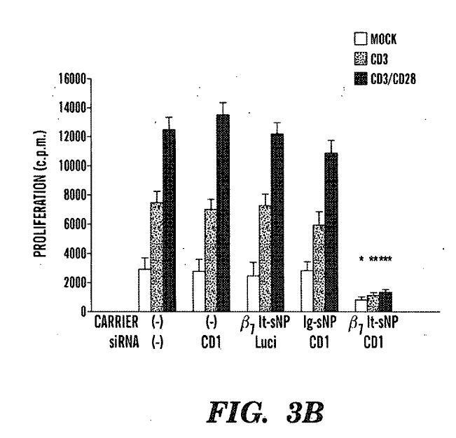

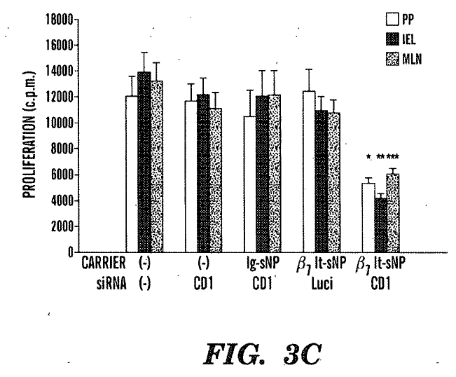

The inventors next tested whether siRNAs against the cell cycle regulator, Cyclin-D1 (CD1) can block the proliferation caused by stimulation with mAbs against CD3 and CD3/CD28. Freshly isolated splenocytes were cultured in the presence of immobilized mAbs (CD3 or CD3/CD28) and 4 hours post plating the cells, they were treated with different formulations of CD1-siRNAs entrapped in Ig-sNP or β₇ It-sNP. Real time RT-PCR performed on mouse primary splenocytes showing mRNA levels of Cyclin D1 are decreased when CD1-siRNAs were delivered through β₇ It-sNP, normalized to the housekeeping gene GAPDH (FIG. 3a). The inventors discovered mRNA levels of CD1 was not decreased when cells were treated with all other controls. Next, the inventors tested if the decrease in the mRNA levels correlates with the function of blocking proliferation. Decreased proliferation (³H-Thymidine incorporation assay) in primary splenocytes treated with β₇ It-sNP entrapping CD1-siRNAs (FIG. 3b) was observed. Other controls did not cause blocking of proliferation, as expected (FIG. 3b).

Next, the inventors assessed whether one can selectively knockdown CD1 in β₇⁺ cells in vivo. To this end, a single intravenous injection of β₇ It-sNP entrapping CD1-siRNAs (2.5 mg/Kg body) or control formulations (having the same siRNAs conc.) showed decrease intrinsic proliferation in cells expressing integrin β₇ such as PP, intraepithelial lymphocytes (IELs), and mesenteric lymph nodes (MLN) assayed by ³H-Thymidine incorporation 2 days post injection (FIG. 3c). None of the other formulations yielded a significant decrease in the proliferation of the cells isolated from PP, IELs, and MLN (FIG. 3c).

### Example 4

### Cyclin D1-siRNAs Delivered by β₇ It-sNP Selectively Reduces Inflammation in an Experimental Colitis Model

Taking together the fact that selective delivery of siRNAs to cells expressing integrin β₇ was demonstrated with fluorescently siRNAs as well as gene silencing with Ku70 and CD1-siRNAs, and that CD1-siRNAs can functionally block the proliferation in vitro and in vivo, the inventors tested the in vivo effect of β₇ It-sNP entrapping CD1-siRNAs in experimental colitis.

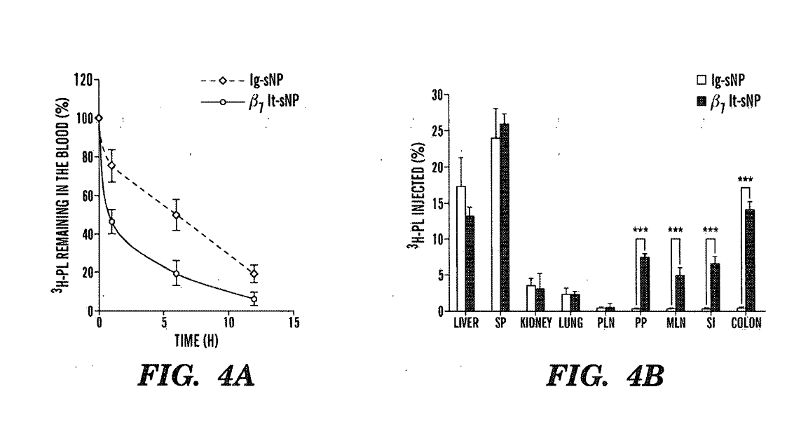

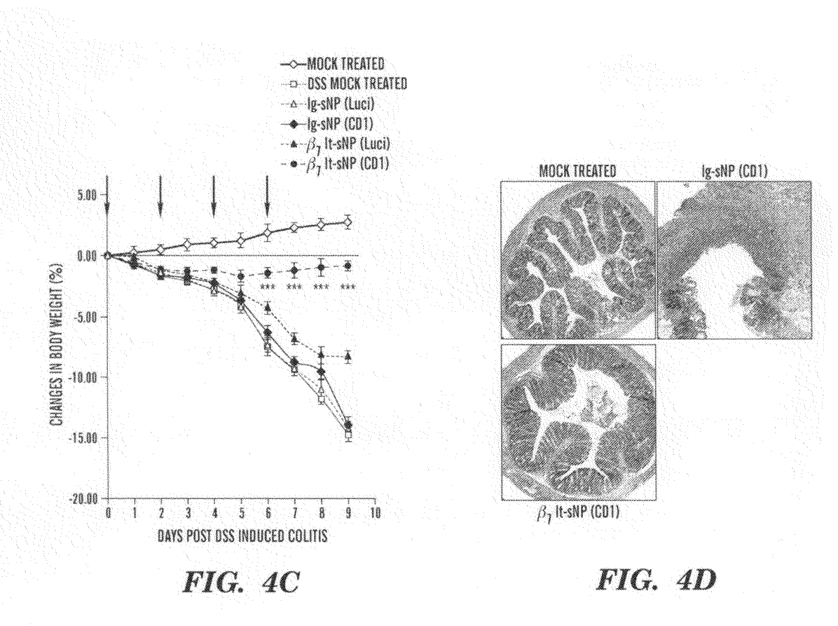

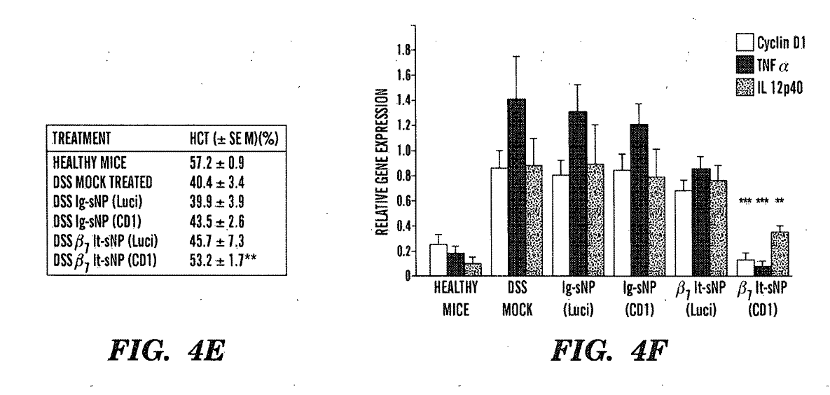

Mice were induced by dextran sulfate sodium (DSS) for 8 days prior to a single intravenous injection with radiolabeled particles as described in Example 2. This model has a number of advantages, including its simplicity and high degree of uniformity of the lesions. With short-term administration, DSS causes a self-limited colitis; with continuous exposure, colitis with chronic features develops. Pharmacokinetics (PK) of labeled β₇ It-sNP revealed a fast elimination from the circulation with a half-life of about an hour (FIG. 4a). Ig-sNP had a slower removal from the circulation with a half-life of close to 7 hours (FIG. 4a). Tissue profile distribution that was performed 12 hours post intravenous injection showed a vast accumulation in organs having β₇⁺ cells such as PP, MLN, SI and the colon. High accumulation was also detected in the liver and spleen (FIG. 4b). Most of the splenocytes are β₇⁺. However, the spleen and liver also tend to host nanoparticles and non-specifically entrap them in Kupper cells in the liver and in the spleen (Lasic et al., 1991; Papahadjopoulos et al., 1991). An indication of it can be seen with Ig-sNP that were also accumulated in the liver and spleen to the same extent (FIG. 4b). The lungs, kidneys, and PLN accumulated lower amounts of both particles without significant advantage of any of the type of particles (i.e. β₇ It-sNP or Ig-sNP).

Based upon the discovery of the PK and tissue distribution profile information in a DSS-induced colitis model, the inventors treated 6 groups of mice by 4 intravenous injections two days apart. The mice groups (n=6/group) included: healthy, untreated mice; DSS-induced-mock treated; Ig-sNP entrapping irrelevant siRNA (luciferase); Ig-sNP entrapping CD1-siRNAs; β₇ It-sNP entrapping luciferase-siRNA and β₇ It-sNP entrapping CD1-siRNAs. All siRNAs were at 2.5 mg/Kg body. Targeting reagents on the nanoparticles were at ˜1 mg/kg body. Mice body weight was monitored daily. The healthy group of mice gains weight in a normal paste (FIG. 4c). DSS-induced colitis group that received only saline decreased its weight and on day 9 and average of 15% decreases in body weight was observed. Both groups that were treated with Ig-sNP (entrapping either luciferase-siRNA or CD1-siRNAs) also lost close to 15% of their body weight by day 9. Mice treated with β₇ It-sNP entrapping luciferase-siRNA lost weight (but less than other groups mentioned above) until day 7, and then started to plateau from day 7-9. This could be explained by the presence of an antibody against integrin β₇ on the surface of the nanoparticle (NP). Anti-integrin therapy using blocking mAbs have been shown to be successful in some pathologies due to blocking of the adhesion and migration of leukocytes into inflamed area. Efalizumab (Raptiva) targeting LFA-1 for the treatment of chronic plaque psoriasis, and natalizumab (Tysabri/Antegren) targeting very late antigen-4 for the treatment of relapsing-remitting multiple sclerosis are examples of anti-integrin therapies (Simmons, 2005). However, adhesion is not the only leukocyte function crucial for maintaining the immune system intact. These antibodies are not able to treat Crohn's disease for example. One other important function of leukocytes is their aberrant proliferation. By blocking this aberrant proliferation and at the same time use anti-adhesion therapy it should be possible to reduce dramatically the inflammation caused in some autoimmune diseases. In fact, it was discovered that mice that were treated with CD1-siRNAs entrapped in β₇ It-sNP had this dual role. Bodyweight of the mice given this treatment reduced their body weight in less then 3% and then after 4 days started to gain back some of the lost weight (FIG. 4c). These results were also demonstrated in histological sections (representatives are presented in FIG. 4d), which demonstrates mice treated with Ig-sNP entrapping CD1-siRNAs where very sick with a massive infiltration of mononuclear cells into the colon tissues (FIG. 4d, Ig-sNP (CD1)), whereas mice treated with CD-1-siRNAs entrapped in β₇ It-sNP looked almost normal (FIG. 4d compare mock treated, to β₇ It-sNP (CD1)) with minimal infiltration into the colon tissues.

In addition to bodyweight changes and histological section, the inventors examined the levels of hematocrit in all mice, as acute colitis might cause severe anemia. The levels of hematocrit are listed in a Table (FIG. 4e), which clearly demonstrates that all DSS-induced mice had anemia to some degree. The inventors discovered that the most severe anemia was observed in untreated but DSS induced mice and mice treated with Ig-sNP (FIG. 4e). Minimal decrease in hematocrit level was detected in the group treated with β₇ It-sNP entrapping CD1-siRNAs, demonstrating almost normal values for the global indicator of inflammation (changes in body weight) and with histological sections that were almost normal.

In addition, the inventors analyzed colon samples from each of the groups using quantitative real-time RT-PCR for mRNA levels of genes that are involved in experimental colitis, namely TNF-α, IL-12p40 and of course CD1 as a positive control for the silencing of the CD1 gene. The inventors discovered a dramatic decrease (p<0.01) in TNF-α and IL12p40 mRNA levels only when mice were treated with β₇ It-sNP entrapping CD1-siRNAs (FIG. 40. In all other treated groups there was no significant difference in the expression levels of these genes normalized to GAPDH (FIG. 4f).

### Example 5

### Binding, Internalization and Silencing of siRNAs Delivered Via Integrin LFA-1-Targeted Stabilized Nanoparticles (LFA-1 It-sNP) to Cells Expressing LFA-1

Mouse anti-human mAb against integrin LFA-1, TS1/22, was used for immobilization on the nanoparticles as described in the experimental section to form LFA-1 It-sNP. Mouse IgG1 was used as an isotype control and was immobilized with the same density on the surface of nanoparticles (Ig-sNP).

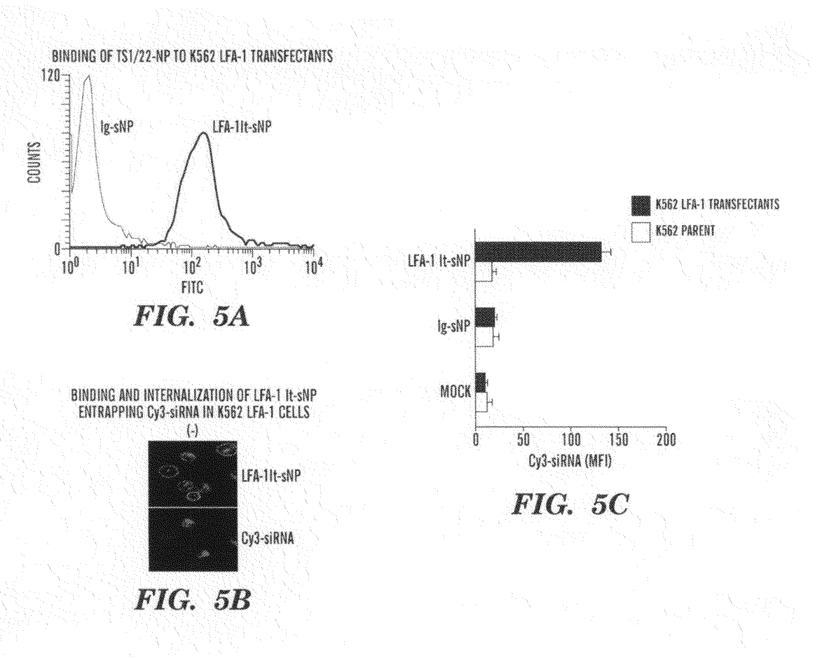

The inventors discovered binding of LFA-1 It-sNP was achieved at 10 μg/ml to cells expressing integrin LFA-1 (K562 LFA-1 transfectants (Peer et al., 2007)) whereas, binding of Ig-sNP at the same concentration did not occur (FIG. 5a).

Next, the inventors used confocal microscopy to investigate the ability of Alexa488-labeled targeting moieties (TS1/22 (LFA-1) or isotype control) to bind and deliver Cy3-siRNA selectively to LFA-1 expressing K562 cells (human transfectants). Four hours after exposure of K562 LFA-1 cells to the fluorescently labeled LFA-1 It-sNP or Ig-sNP, it was discovered that Alexa488-LFA-1It-sNP were distributed to both the plasma membrane and internal punctate structures, whereas Cy3-siRNA was predominantly intracellular, colocolized with the Alexa488-LFA-1It-sNP (FIG. 5b). Minimal Cy3-siRNA uptake was discovered when delivery was via Ig-sNP (data not shown). The inventors confirmed the specificity of cy3-siRNA into K562 LFA-1 transfectant by examining the uptake of Cy3-siRNA using Ig-sNP and LFA-1 It-sNP in both parent K562 cells (not expressing LFA-1) and the K562 transfectants (expressing LFA-1) (FIG. 5c). The inventors demonstrate that K562 LFA-1 cells specifically uptake Cy3-siRNA that was delivered via LFA-1It-sNP, but not with Ig-sNP (FIG. 5c). Cy3-siRNA without any delivery system did not significantly accumulated in either type of cells. Parent K562 cells did not uptake more siRNA delivered via LFA-1 It-sNP compare to Ig-sNP or free siRNA (FIG. 5c).

### Example 6

### Delivery of Poorly Soluble Drug to K562 LFA-1 Cells Via LFA-1 It-sNP

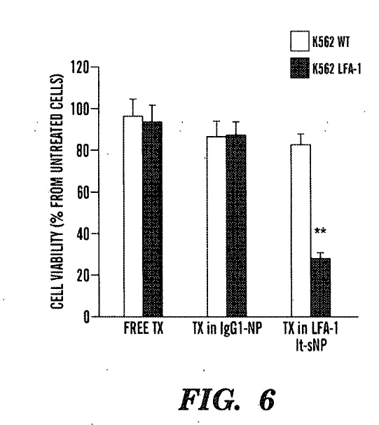

Paclitaxel, which is a poorly soluble chemotherapy agent, was entrapped in LFA-1It-sNP and separately in Ig-sNP in the lipid phase as described in the methods section above. Encapsulation efficiency was 95±9% as determined by radioactivity (also detailed in the methods section). K562 WT and LFA-1 transfectants were treated with 3 formulations of paclitaxel (all include paclitaxel (TX) at 50 nM). Cells were treated for 4 hours followed by washing the cells with media and replacing the media-containing drug with drug free-media. Then cells were culture for additional 48 hours prior to a cell viability assay (MTT). The inventors clearly demonstrate, as shown in FIG. 6, that only cells expressing the integrin LFA-1 could significantly (p<0.03) take up paclitaxel when it was delivered via LFA-1 It-sNP (FIG. 6). A decrease in cell viability was not observed in the parent K562 cells in either of the formulations, convincingly demonstrating that the LFA-1 It-sNP delivered paclitaxel efficiently and specifically only to cells expressing LFA-1. Non-specific uptake in this short exposure time was minimal (FIG. 6).

### Example 7

### Dual Entrapment of Lipophilic and Hydrophilic Drugs Delivered with Anti-Integrin-Stabilized Nanoparticles Decreases Cell Death

The inventors then asked whether a combinational treatment of dual drugs working on the cytoskeleton (paclitaxel) and on blocking the proliferation (CD1-siRNAs) increase cell death in this same context.

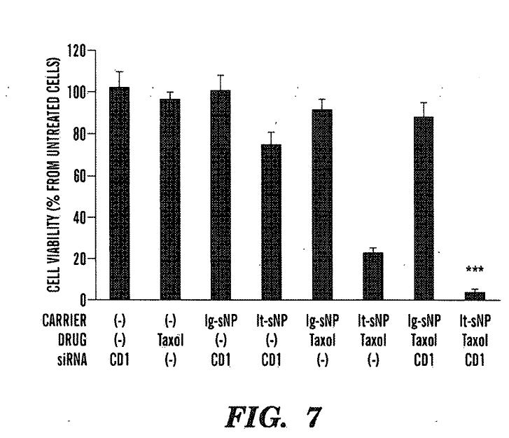

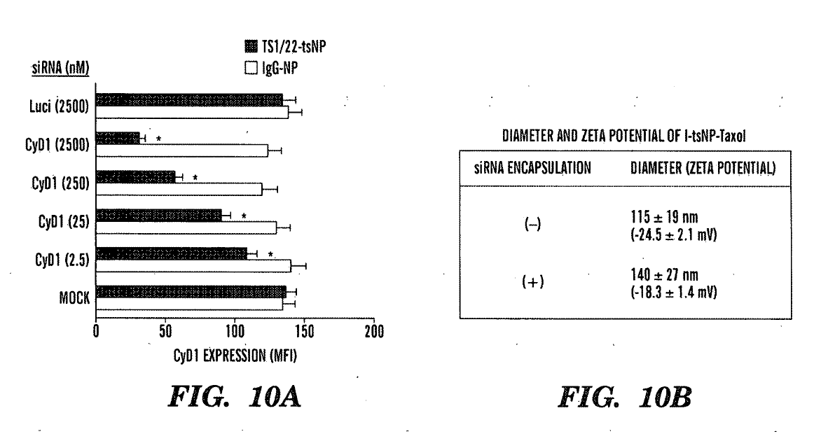

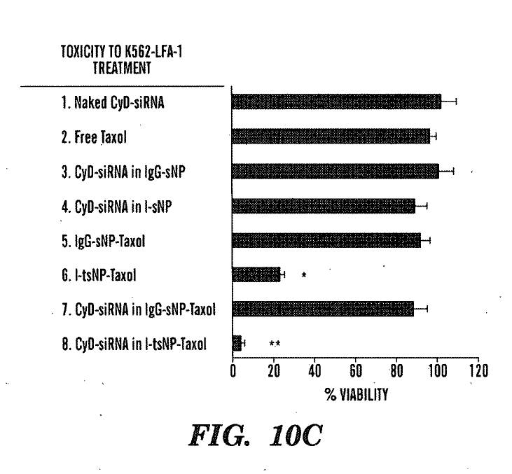

Paclitaxel was entrapped in LFA-1It-sNP or in Ig-sNP in the lipid phase as described. SiRNAs were first condensed by protamine and entrapped upon lyophilization of the immunoparticles as described in FIG. 8. FIG. 9 represents a schematic illustration of dual entrapment. A combinational treatment was given to K562 LFA-1 expressing cells using a short exposure time (4 hours) as described in the experimental section. Despite a short exposure time, the inventors discovered reduce the cell viability of K562 LFA-1 treated with LFA-1It-sNP entrapping CD1-siRNAs, as well as a treatment with paclitaxel in It-sNP, clearly demonstrating a combinational treatment had the most significant effect (FIG. 7 and FIG. 10C). All other controls did not dramatically decrease the cell viability as compared to the combined treatment with Paclitaxel and CD1-siRNAs entrapped in LFA-1It-sNP.

The inventors next demonstrated that dual entrapment of insoluble agent (or lipophilic drug) such as TAXOL® and a soluble agent (i.e. a hydrophilic drug) such as an RNAi could be delivered with anti-integrin-stabilized nanoparticles to cells to decrease cell death. The inventors demonstrate in FIG. 10A the silencing of CyclinD1 gene expression by siRNA-CyclinD1 using a nanoparticle which has entrapped a soluble agent such siRNA to CyclinD1 only. The inventors demonstrate that a nanoparticle entrapping both a soluble agent such as siRNA-CyclinD1 and an insoluble agent such as TAXOL® does not significantly change the nanoparticle diameter or zeta potential relative to a formulation with siRNA alone, as shown in FIG. 10B. The Zeta potential is one measurement of a nanoparticle characteristics, and is the potential between the particle surface and an electroneutral medium. If Zeta potential is near zero it indicates that the nanoparticles will aggregate. Conversely, if the Zeta potential is highly positive or if the zeta potential is negative, then it is likely that no nanoparticle aggregation will occur, except in circumstances of very high zeta potential values (>20 mV), which have been shown to cause toxicity in the body. Zeta potentials that are mildly negative are tolerated when administered to the body.

### REFERENCES

The references cited herein and throughout the application are incorporated herein by reference.
Anderson, M. E., and Siahaan, T. J. (2003). Mechanism of binding and internalization of ICAM-1-derived cyclic peptides by LFA-1 on the surface of T cells: a potential method for targeted drug delivery. Pharm Res 20, 1523-1532.
Coffey, G. P., Stefanich, E., Palmieri, S., Eckert, R., Padilla-Eagar, J., Fielder, P. J., and Pippig, S. (2004). In vitro internalization, intracellular transport, and clearance of an anti-CD11a antibody (Raptiva) by human T-cells. J Pharmacol Exp Ther 310, 896-904.
de Fougerolles, A. R. (2003). Integrins in immune and inflammatory diseases. In I Domains in Integrins, D. Gullberg, ed. (Georgetown, Tex., Plenum Publishers), pp. 165-177.
Demoy, M., Andreux, J. P., Weingarten, C., Gouritin, B., Guilloux, V., and Couvreur, P. (1999). Spleen capture of nanoparticles: influence of animal species and surface characteristics. Pharm Res 16, 37-41.
Fabbri, M., Di Meglio, S., Gagliani, M. C., Consonni, E., Molteni, R., Bender, J. R., Tacchetti, C., and Pardi, R. (2005). Dynamic Partitioning into Lipid Rafts Controls the Endo-Exocytic Cycle of the {alpha}L/{beta}2 Integrin, LFA-1, during Leukocyte Chemotaxis. Mol Biol Cell 16, 5793-5803.
Feagan, B. G., Greenberg, G. R., Wild, G., Fedorak, R. N., Pare, P., McDonald, J. W., Dube, R., Cohen, A., Steinhart, A. H., Landau, S., Aguzzi, R. A., Fox, I. H., and Vandervoort, M. K. (2005). Treatment of ulcerative colitis with a humanized antibody to the alpha4beta7 integrin. N Engl J Med 352, 2499-2507.
Hornung, V., Guenthner-Biller, M., Bourquin, C., Ablasser, A., Schlee, M., Uematsu, S., Noronha, A., Manoharan, M., Akira, S., de Fougerolles, A., Endres, S., and Hartmann, G. (2005). Sequence-specific potent induction of IFN-alpha by short interfering RNA in plasmacytoid dendritic cells through TLR7. Nat Med 11, 263-270.
Lasic, D. D., Martin, F. J., Gabizon, A., Huang, S. K., and Papahadjopoulos, D. (1991). Sterically stabilized liposomes: a hypothesis on the molecular origin of the extended circulation times. Biochim Biophys Acta 1070, 187-192.
Luster, A. D., Alon, R., and von Andrian, U. H. (2005). Immune cell migration in inflammation: present and future therapeutic targets. Nat Immunol 6, 1182-1190.
Margalit R., A. R., Linenberg M., Rubin I., Roseman T. J. and Wood R. W. (1991). Liposomal drug delivery: thermodynamic and chemical kinetic considerations. Journal of controlled release 17, 285-296.
Morrison, W. R. (1964). A Fast, Simple and Reliable Method for the Microdetermination of Phosphorus in Biological Materials. Anal Biochem 7, 218-224.
Neurath, M. F., Weigmann, B., Finotto, S., Glickman, J., Nieuwenhuis, E., lijima, H., Mizoguchi, A., Mizoguchi, E., Mudter, J., Galle, P. R., Bhan, A., Autschbach, F., Sullivan, B. M., Szabo, S. J., Glimcher, L. H., and Blumberg, R. S. (2002). The transcription factor T-bet regulates mucosal T cell activation in experimental colitis and Crohn's disease. J Exp Med 195, 1129-1143.
Papahadjopoulos, D., Allen, T. M., Gabizon, A., Mayhew, E., Matthay, K., Huang, S. K., Lee, K. D., Woodle, M. C., Lasic, D. D., Redemann, C., and et al. (1991). Sterically stabilized liposomes: improvements in pharmacokinetics and antitumor therapeutic efficacy. Proc Natl Acad Sci USA 88, 11460-11464.
Park, E. J., Takahashi, I., Ikeda, J., Kawahara, K., Okamoto, T., Kweon, M. N., Fukuyama, S., Groh, V., Spies, T., Obata, Y., Miyazaki, J., and Kiyono, H. (2003). Clonal expansion of double-positive intraepithelial lymphocytes by MHC class I-related chain A expressed in mouse small intestinal epithelium. J Immunol 171, 4131-4139.
Peer, D., and Margalit, R. (2000). Physicochemical evaluation of a stability-driven approach to drug entrapment in regular and in surface-modified liposomes. Arch Biochem Biophys 383, 185-190.
Peer, D., Zhu, P., Carman, C. V., Lieberman, J., and Shimaoka, M. (2007). Selective gene silencing in activated leukocytes by targeting siRNAs to the integrin lymphocyte function-associated antigen-1. Proc Natl Acad Sci USA 104, 4095-4100.
Peracchia, M. T., Fattal, E., Desmaele, D., Besnard, M., Noel, J. P., Gomis, J. M., Appel, M., d'Angelo, J., and Couvreur, P. (1999). Stealth PEGylated polycyanoacrylate nanoparticles for intravenous administration and splenic targeting. J Control Release 60, 121-128.
Sapra, P., and Allen, T. M. (2004). Improved outcome when B-cell lymphoma is treated with combinations of immunoliposomal anticancer drugs targeted to both the CD19 and CD20 epitopes. Clin Cancer Res 10, 2530-2537.
Simmons, D. L. (2005). Anti-adhesion therapies. Curr Opin Pharmacol 5, 398-404.
Song, E., Zhu, P., Lee, S. K., Chowdhury, D., Kussman, S., Dykxhoorn, D. M., Feng, Y., Palliser, D., Weiner, D. B., Shankar, P., Marasco, W. A., and Lieberman, J. (2005). Antibody mediated in vivo delivery of small interfering RNAs via cell-surface receptors. Nat Biotechnol 23, 709-717.
Sydora, B. C., Wagner, N., Lohler, J., Yakoub, G., Kronenberg, M., Muller, W., and Aranda, R. (2002). beta? Integrin expression is not required for the localization of T cells to the intestine and colitis pathogenesis. Clin Exp Immunol 129, 35-42.

1. A composition for delivering at least one insoluble agent and at least one soluble agent to a target cell comprising,
(a) a targeting moiety that selectively binds one or more cell surface markers on the surface of the target cell;
(b) a carrier particle associated with the targeting moiety, wherein the carrier particle has a lipid phase and an aqueous phase;
(c) an insoluble agent entrapped in the lipid phase of the carrier particle; and
(d) a soluble agent entrapped in the aqueous phase of the carrier particle.

2. The composition of claim 1, wherein the targeting moiety comprises an antibody or integrin ligand, or functional fragments or variants thereof.

3. The composition of claim 1, wherein the targeting moiety comprises a scFv, an IgG, Fab′, F(ab′)2, or a recombinant bivalent scFv, or fragments thereof.

4. The composition of claim 1, wherein the carrier particle comprises a liposome or other lipid or non-lipid carrier or a functional fragment thereof.

5. The composition of claim 4, wherein the liposome is unilamellar with a first layer comprising glycosaminoglycan hyaluronan (HA) covalently linked to phosphatidylethanolamine therein, and a second layer comprising specific antibodies covalently attached to the HA of the first layer.

6. The composition of claim 1, wherein the insoluble agent is selected from the group consisting of a lipophilic RNAi, antibiotics, immunosuppressants, antibacterial agents, chemotherapeutic agents, paclitaxel, platinum-based drugs, anthracyclines, mitomycin C, compounds of the quinolone family of synthetic antibacterial compounds, enoxacin, ciprofloxacin, ofloxacin, norfloxacin, and difloxacin and combinations thereof.

7. The composition of claim 1, wherein the soluble agent is selected from the group consisting of an RNA interference (RNAi) molecule, a small molecule, a polypeptide, lipophilic agent, hydrophobic agent, antibody or a functional fragment thereof.

8. The composition of claim 7, wherein the RNA interference molecule is selected from the group consisting of siRNA, dsRNA, stRNA, shRNA, miRNA, and combinations thereof.

9. The composition of claim 1, wherein the target cell is a mammalian cell.

10. The composition of claim 1, wherein the target cell is a human cell.

11. The composition of claim 1 which targets a leukocyte
wherein the targeting moiety selectively binds one or more integrins on the surface of a leukocyte.

12. (canceled)

13. The composition of claim 11, wherein the targeting moiety selectively binds to an integrin in its activated conformation.

14. The composition of claim 11 wherein the integrin is selected from the group consisting of LFA-1 (αLβ2), Mac-1 (αMβ2), p150.95 (αXβ2), αDβ2, VLA-4 (α4β1), and β7 (α4β7 and αEβ7).

15. The composition of claim 11 wherein the integrin can bind an integrin ligand selected from the group consisting of ICAM-1, ICAM-2, ICAM-3, VCAM-1, MAdCAM-1, E-cadherin, JAM-1, JAM-2 and JAM-3.

16. The composition of claim 11, wherein the integrin is LFA-1 and the targeting moiety comprises an antibody or functional fragment thereof, which binds to the locked open I domain of LFA-1, or binds to the leg domain of the β2 subunit of LFA-1 (αLβ2) or integrin β7.

17. (canceled)

18. (canceled)

19. The composition of claim 11, wherein the targeting moiety comprises an antibody or functional fragment thereof, which binds non-selectively to low affinity and high affinity LFA-1, Mac-1 and integrin β7.

20.-38. (canceled)

39. A method for delivery of at least one insoluble agent and at least one soluble agent to a leukocyte present in a subject, comprising:
administering to a subject a composition of claim 1
wherein the composition contacts the leukocyte to deliver the at least one insoluble agent and at least one soluble agent to the leukocyte.

40. The method of claim 39, wherein the composition is selective for activated leukocytes.

41.-64. (canceled)

65. The composition of claim 1 which targets an endothelial cell, wherein the
targeting moiety selectively binds one or more integrin ligands on the surface of the endothelial cell.

66. The composition of claim 65, wherein the integrin ligand is selected from the group consisting of ICAM-1, ICAM-2, ICAM-3, VCAM-1, MAdCAM-1, E-Cadherin, JAM-1, JAM-2 and JAM-3.

67. The composition of claim 65, wherein the integrin ligand binds to an integrin present on the surface of leukocytes, wherein an integrin present on the surface of a leukocyte is selected from the group consisting of LFA-1 (αLβ2), Mac-1 (αMβ2), p150.95 (αXβ2), αDβ2, VLA-4 (α4β1), and β7 (α4β7 and αEβ7).

68.-99. (canceled)

100. A method for delivery of at least one insoluble agent and at least one soluable agent to an endothelial cell present in a subject, comprising administering to a subject a composition of claim 65, wherein the composition contacts the endothelial cell to deliver the agent to the endothelial cell.

101. The method of claim 39, wherein the subject is a human.

102. The method of claim 100, wherein the subject is a human.
